(19) United States
(12) Patent Application Publication
(10) Pub. No.: US 2006/0292599 A1
(43) Pub. Date: Dec. 28, 2006

(54) Methods to detect lineage-specific cells

(75) Inventors: Jerome Ritz, Lincoln, MA (US); Catherine J. Wu, Cambridge, MA (US); Ephraim P. Hochberg, (US)

Correspondence Address: FOLEY HOAG, LLP;PATENT GROUP, WORLD TRADE CENTER WEST, 155 SEAPORT BLVD, BOSTON, MA, 02110, US

(73) Assignee: Dana-Farber Cancer Institute, Inc., Boston, MA (US)

(21) Appl. No.: 11/389,543

(22) Filed: Mar. 24, 2006

Related U.S. Application Data
(63) Continuation of application No. PCT/US04/31524, filed on Sep. 24, 2004.
(60) Provisional application No. 60/506,221, filed on Sep. 25, 2003.
(60) Provisional application No. 60/509,594, filed on Oct. 8, 2003.

(51) Int. Cl. C12Q 1/68

(52) U.S. Cl. 435/6

(57) ABSTRACT

The present invention is based, at least in part, on methods to identify and quantify lineage-specific cells. The present invention provides methods of detecting lineage-specific cells in a biological sample, and monitoring the effectiveness of progenitor cell transfer in a subject. The invention further provides methods of determining an effective dose of progenitor cell transfer in a subject and methods of quantifying progenitor cell transfer in a subject. Additionally, methods are provided to identify allelic variants in lineage-specific cells.

### RELATED APPLICATIONS

This application is continuation of PCT/US04/31524, which was filed on Sep. 24, 2004, which claims the benefit of U.S. Provisional Application Ser. No. 60/506,221, filed Sep. 25, 2003, and U.S. Provisional Application Ser. No. 60/509,594, filed Oct. 8, 2003, the entire contents of each of which are incorporated herein by this reference.

### GOVERNMENT FUNDING

Work described herein was supported, at least in part, by National Institutes of Health (NIH) under grants KO8 HL04293 and AI29530. The U.S. government therefore may have certain rights in this invention.

### BACKGROUND OF THE INVENTION

Transplantation of stem cells is a curative option for many hematologic malignancies. Transplantation of cells that have been genetically engineered to replace cells that produce either no protein or defective protein as the result of inherited or idiopathic diseases or disorders, e.g., Cystic Fibrosis, is also proving to be feasible. However, for any cell transplantation to be successful, it is critically important to quantify the level of engraftment of donor cells or transgenic cells relative to recipient or defective cells. Current methods for measuring the relative numbers of donor versus recipient cells are based on DNA polymorphisms that distinguish recipient from donor. These methods provide an accurate assessment of engraftment of donor cells, however, these methods do not provide an assessment of the functional capacity of the donated cells and do not directly examine engraftment of specific cell lineages following transplant. Moreover, these methods require prior purification of cellular subsets which often results in samples that are not representative of the original sample.

For example, non-myeloablative conditioning regimens for allogenic stem cell transplantation are now commonly used in the treatment of subjects with hematologic malignancies, and since this treatment often results in the establishment of mixed hematopoietic chimerism, a similar approach is also useful in the treatment of nonmalignant disorders, such as sickle cell disease and thalassemia major. In order to apply this approach for these diseases, it is necessary to determine the levels of donor erythropoiesis required to correct hemolysis and reconstitute immune function in order to ameliorate disease symptoms and minimize end-organ damage. The current methods for measuring cell chimerism are effective and accurately measure donor cell engraftment, but do not quantify the relative contributions of recipient and donor erythropoiesis and/or immune function following transplant.

### SUMMARY OF THE INVENTION

The present invention is based, at least in part, on methods for measuring functional cell engraftment of lineage-specific cells. Accordingly, the invention provides a method of detecting lineage-specific cells in a biological sample by identifying lineage-specific mRNA in a biological sample.

In another aspect of the invention, the present invention provides methods of monitoring the effectiveness of progenitor cell transfer in a subject, comprising the step of identifying lineage-specific mRNA in the subject.

In another aspect, the invention provides methods of detecting lineage-specific cells in a biological sample comprising the step of identifying at least one allelic variant in lineage-specific mRNA in the sample. In one embodiment, the at least one allelic variant is in a gene selected from the genes listed in Tables 4, 6, 7, and 8.

In yet another aspect, the invention features a method of detecting lineage-specific cells in a biological sample comprising the step of identifying at least one single nucleotide polymorphism (SNP) in lineage-specific mRNA in said sample, thereby detecting lineage-specific cells in said sample. In one embodiment, the at least one SNP is in a gene selected from the genes listed in Tables 4, 6, 7, and 8. In yet another embodiment, the at least one SNP is selected from the group listed in Table 9 (see below). For example, the SNP(s) may be contained within a β-globin gene.

In another embodiment, the lineage-specific cells are hematopoietic cells, e.g., erythroid cells, lymphoid cells, or myeloid cells.

In still another embodiment, the lineage-specific mRNA is identified by sequencing. In a further embodiment, the sequencing is pyrosequencing. In another embodiment, the lineage-specific mRNA is identified by an array-based method.

Another aspect of the invention features a method of quantifying donor and recipient lineage-specific cells in a subject following progenitor cell transfer comprising the steps of obtaining a biological sample from said subject following progenitor cell transfer; and identifying and quantifying the presence of one or more donor-derived allelic variants and the presence of one or more recipient-derived allelic variants.

Still another aspect of the invention features a method of detecting lineage-specific chimerism of a subject following progenitor cell transfer comprising the steps of obtaining a biological sample from said subject following progenitor cell transfer; and identifying and quantifying the presence of one or more donor-derived lineage-specific allelic variants and the presence of one or more recipient-derived lineage-specific allelic variants. In one embodiment, the biological sample is blood. In another embodiment, the biological sample is bone marrow.

In still another embodiment, the allelic variants are contained within a lineage-specific gene. In a further embodiment, the lineage-specific gene is selected from the group of genes listed in Tables 4, 5, 6, and 7.

In one embodiment, the allelic variants are SNPs. In another embodiment, the SNPs are selected from the group consisting of those SNPs listed in Table 9. In yet another embodiment, the allelic variants are identified by an array-based method.

In one embodiment, the lineage-specific allelic variants are expressed by a lineage-specific cell selected from the group consisting of erythroid, lymphoid, or myeloid cells.

In one embodiment, the subject is suffering from a disease or disorder. In another embodiment, the disease or disorder is associated with reduced levels of β-globin mRNA. In still another embodiment, the disease or disorder is selected from the group consisting of: hemoglobinopathies, hemolytic anemia, hereditary elliptocytosis, hereditary stomatocytosis, Chronic Granulomatous Disease, Chediak-Higashi syndrome, myelodysplasia, acute erythroleukemia, Kostmann's syndrome, infant malignant osteopetrosis, severe combined immunodeficiency, Wiskott-Aldrich syndrome, aplastic anemia, Blackfan Diamond anemia, Gaucher's disease, Hurler's syndrome, Hunter's syndrome, infantile metachromatic leukodystrophy, autoimmune disorders, osteogenesis imperfecta, myocardial injury-syndromes, Cystic Fibrosis, hemophilia, Gaucher's disease, diabetes mellitus, organ failure or injury, e.g., cardiac, brain, lung, liver, renal, prostate or pancreas organ failure or injury, and cancers associated with oncogenes, e.g., breast, prostate, or colon cancer.

In another embodiment, the disease of disorder is a cognitive or neurodegenerative disease or disorder, e.g., Alzheimer's disease, stroke, dementias related to Alzheimer's disease (such as Pick's disease), Parkinson's and other Lewy diffuse body diseases, senile dementia, Huntington's disease, Gilles de la Tourette's syndrome, musculoskeletal diseases, multiple sclerosis, amyotrophic lateral sclerosis, progressive supranuclear palsy, epilepsy, or Jakob-Creutzfieldt disease.

In still another embodiment, the progenitor cell is a stem cell or a transgenic cell.

Another aspect of the invention provides a method of quantifying progenitor cell transfer in a subject comprising the steps of: (a) obtaining a biological sample prior to said progenitor cell transfer; (b) obtaining a biological sample following said progenitor cell transfer; (c) identifying and quantifying lineage-specific allelic variants in said biological sample obtained in step (a); (d) identifying and quantifying lineage-specific allelic variants in said biological sample obtained in step (b); and (e) comparing the quantity of progenitor cell transfer from step (c) and step (d) thereby quantifying progenitor cell transfer in a subject.

In still another aspect of the invention, a method is provided to determine an effective dose of progenitor cell transfer in a subject comprising the steps of: (a) obtaining a biological sample prior to said progenitor cell transfer; (b) obtaining a biological sample following said progenitor cell transfer, (c) identifying and quantifying lineage-specific allelic variants in said biological sample obtained in step (a), thereby quantifying progenitor cell transfer in said biological sample; (d) identifying and quantifying lineage-specific allelic variants in said biological sample obtained in step (b), thereby quantifying progenitor cell transfer in said biological sample; and (e) comparing the quantity of progenitor cell transfer from step (c) and step (d) to therapy outcome, thereby determining an effective dose of progenitor cell transfer.

In one aspect, a method is provided for detecting lineage-specific cells in a biological sample, comprising the steps of: (a) isolating mRNA from said biological sample; (b) reverse transcribing cDNA from said mRNA; (c) amplifying said cDNA; and (d) identifying lineage-specific cDNA in the sample.

Another aspect of the invention provides a method of detecting lineage-specific cells in a biological sample, comprising the steps of: (a) ascertaining at least one lineage-specific allelic variant in a target sequence; (b) isolating mRNA from said biological sample; (c) reverse transcribing cDNA from said mRNA; (d) amplifying said at least one allelic variant from said cDNA by a template dependent process; and (e) identifying the at least one lineage-specific allelic variant in step (a) in said sample, thereby detecting lineage-specific cells in a biological sample.

In one embodiment, the amplification of said cDNA comprises the amplification two or more allelic variants.

In one embodiment, the at least one allelic variant is in a gene selected from the genes listed in Tables 4, 6, 7, and 8.

In one embodiment, the amplification of cDNA amplifies a polymorphic region of a gene or fragment thereof selected from the genes listed in Tables 4, 6, 7, and 8. In another embodiment, the amplification of cDNA amplifies a β-globin gene or fragment thereof. In a further embodiment, the amplification of the β-globin gene or fragment thereof utilizes the primers set forth as SEQ ID No.: 3 and SEQ ID NO: 5. In another embodiment, the amplification of the β-globin gene or fragment thereof utilizes the primers set forth as SEQ ID No.: 6 and SEQ ID NO: 8.

In still another aspect of the invention, a kit is provided to identify lineage-specific cells in a biological sample comprising, (a) primers for the amplification of lineage-specific mRNA; and (b) instructions for use of said primers to identify lineage-specific cells in said biological sample.

Another aspect of the invention provides a kit to monitor the effectiveness of progenitor cell transfer comprising, (a) primers for the amplification of lineage-specific mRNA, and (b) instructions for use of said primers to monitor the effectiveness of progenitor cell transfer.

In one embodiment, the primers for the amplification of lineage-specific mRNA are set forth as SEQ ID No.: 3 and SEQ ID NO: 5. In another embodiment, the primers for the amplification of lineage-specific mRNA are set forth as SEQ ID No.: 6 and SEQ ID NO: 8.

In still another embodiment, the lineage-specific cells are hematopoietic cells, e.g., erythroid cells, lymphoid cells, and myeloid cells. In another embodiment, the progenitor cell is a stem cell. In a further embodiment, the progenitor cell is a transgenic cell.

A further aspect of the invention features a method for determining the clinical outcome of a progenitor cell transfer in a subject comprising obtaining a biological sample from said subject and identifying lineage-specific mRNA in said biological sample, wherein a substantial amount of donor-derived lineage-specific allelic variants selected from the group in Table 7 is an indication of poor clinical outcome and a substantial amount of recipient-derived lineage-specific allelic variants selected from the group in Table 7 is an indication of favorable clinical outcome.

Yet another aspect of the invention features a method for determining immune reconstitution in a subject following progenitor cell transfer comprising the steps of obtaining a biological sample from said subject and identifying the identify of at least one lineage-specific allelic variant in said biological sample, to thereby determine immune reconstitution in a subject.

### BRIEF DESCRIPTION OF THE DRAWINGS

FIG. 1 is a schematic for the detection and quantitation of erythroid-lineage specific chimerism using RNA pyrosequencing compared with the assessment of genomic DNA chimerism by DNA pyrosequencing.

FIG. 2 is an image that demonstrates that sequence specific PCR primers for the β-globin gene amplify genomic DNA (GDNA) from cells of various lineages, including peripheral blood mononuclear cells (PBMC) and EBV-transformed B cell lines, but only amplify cDNA derived from erythroid lineage cells. GAPDH was amplified as a positive control.

FIGS. 3A-3B depict pyrograms of β-globin gene mutations and polymorphisms. (A). Pyrograms generated from sequencing the complementary strand of the β-globin gene around the sickle mutation (actual sequence: C A/T CAGGA). Pyrograms of homozygous sickle cell disease (A/A), sickle cell trait (A/T) and normal donor (T/T) are shown. (B). Pyrograms generated from sequencing the complementary strand of the β-globin gene surrounding the H3H polymorphism (actual sequence: G G/A TGCACC).

FIGS. 4A-4B are graphs that depict the quantitative assessment of chimerism by pyrosequencing. (A). Plasmids encoding normal or sickle β-globin were mixed in varying ratios and amplified with primers specific for β-globin cDNA. The expected input frequency and measured output by pyrosequencing were highly correlated (r²<0.968). This experiment was repeated 4 times, as represented by the different symbols. (B). cDNA derived from a normal donor homozygous for β-globin H3H polymorphism was mixed in varying ratios with cDNA of another normal donor heterozygous at the same loci, and the mixtures were amplified with primers specific for the region around the β-globin H3H polymorphism. Input mixtures correlated well with output percentages measured by pyrosequencing (r²<0.9487). This experiment was repeated 3 times.

FIG. 5 shows graphs that depict the changes in blood hemoglobin following transplantation measured by hemoglobin electrophoresis. Percentages of hemoglobin-A1 and hemoglobin-S were determined by hemoglobin electrophoresis for Subjects 1 and 2 during the first 3 months after transplantation. The shaded region represents the period during which normal donor RBCs were transfused. Results are compared to donor values indicated at the far right.

FIG. 6 shows graphs that depict a comparison of hematopoietic DNA chimerism with erythroid lineage RNA chimerism following transplantation. Hematopoietic DNA chimerism was determined by conventional STR analysis and DNA pyrosequencing for the sickle mutation. Erythroid chimerism was determined by pyrosequencing for the sickle mutation in β-globin RNA.

FIG. 7 shows graphs that depict serial measurements of donor chimerism in genomic DNA and β-globin RNA after HSCT.

FIG. 8 depicts genes in the merged dataset derived from expression datasets of normal B, T, NK, macrophage and blood-derived dendritic cells, clustered on the basis of RNA expression levels (darkest gray-high expression levels; lightest gray=low expression levels) and each hematopoietic cell subset could be clearly distinguished on the basis of expression of a defined set of genes.

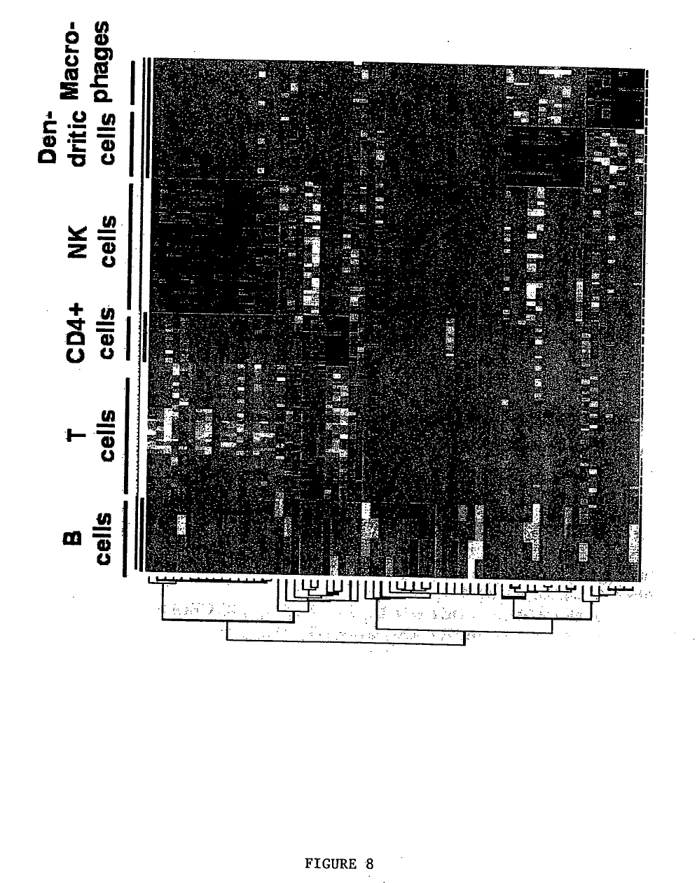

### DETAILED DESCRIPTION OF THE INVENTION

The present invention is based, at least in-part, on the discovery of methods to detect lineage-specific cells. In-one embodiment, the present invention provides methods to accurately identify, detect, and quantify a functional cell, e.g., a lineage-specific cell, e.g., a transfected cell or a stem cell. For example, the present invention provides methods to identify, detect, and quantify the engraftment of donor cells that give rise to hematopoietic and non-hematopoietic lineages following stem cell transplantation. The present invention also provides methods of determining the clinical outcome of a subject following progenitor cell transfer.

The transplantation of autologous or allogenic bone marrow-derived stem cells and/or hematopoietic stem cells, e.g., bone marrow transplantation (BMT), has been used extensively to treat subjects with malignant hematological diseases or disorders, e.g., myeloid and lymphoid malignancies, e.g., as classified by World Health Organization (WHO), following myeloablative combinations of high dose chemo-radiotherapy. The transplantation of stem cells was considered a means to overcome the myelotoxic effects of the chemo-radiotherapy providing reconstitution of hematopoiesis. Over the years, however, it was recognized that there was a persistence and/or reappearance of the recipient hematopoietic cells in addition to the donor hematopoietic cells following myeloablative allogenic BMT resulting in hematopoietic chimerism. It has become clear, in fact, that the immune reaction between donor-derived immunocompetent cells and host-type tumor cells was an important therapeutic event and accounted for better anti-tumor effects, e.g., graft-versus-leukemia (GVL) and graft-versus-tumor (GVT) effects, induced by allogenic BMT compared to autologous BMT (reviewed in Khouri, I., et al. (2002) Cancer Treat. Res. 110:137).

Myeloablative stem cell transplantation is a curative option for many hematologic malignancies but is usually reserved for younger subjects without serious comorbid conditions, especially graft versus host disease (GVHD) and serious bacterial, viral, and fungal infections due to prolonged myelosuppression and immuno-suppression with conventional conditioning regimens (Ringden, O., et al. (1993) J. Am. Med. Assoc. 270:57). A strategy utilizing a less intensive, non-myelosuppressive preparative regimen that is sufficiently immuno-suppressive to prevent graft rejection and allow engraftment of hematopoietic stem cells and the establishment of hematopoietic chimerism has been developed and used successfully to treat hemotologic malignancies in subjects ineligible for high dose chemotherapy or radiation. This therapy is referred to as “non-myeloablative stem cell transplantation” (NST) or “mini-transplants”, as used herein (Krause, D. S., et al. (2001) Cell 105:369-377; Jiang, Y., et al. (2002) Nature 418:41-49; Wagers, A. J., et al. (2002) Science 297:2256-2259; Sykes, et al., U.S. Pat. No. 6,558,662). Since this method is associated with fewer complications than traditional BMT conditioning regimens and still allows the development of hematopoietic chimerism, this approach is also useful in the treatment of nonmalignant hematological disorders (Reya, T., et al. (2001) Nature 414:105-111) and is sufficient to correct genetic defects of hematopoiesis such as, for example, hemoglobinopathies, e.g., sickle cell syndromes and thalassemia syndromes, or other severe metabolic diseases or disorders, such as Hunters or Hurler's Syndromes (Yeager et al. (2002) Ann Hematol 81: Suppl S16-9), and defects in hematopoiesis or immune function (Balkwill, F. & Mantovani, A. (2001) Lancet 357:539-545; Houghton, J. M., et al. (2002) J. Gastro. Hep. 17:495-502; Schmidt P. H., et al. (1999) Lab. Invest. 79:639-646; Fox, J. G., et al. (2002) Cancer Res. 62:696-702).

In order that non-myeloablative stem cell transplantation be successful for the treatment of nonmalignant hematological diseases and disorders, e.g., hemoglobinopathies, e.g., sickle cell syndromes and thalassemia syndromes, severe metabolic diseases and disorders, defects in hematopoiesis or immune function, cognitive and neurodegenerative diseases and disorders, and cancer, it is critically important to quantify the level of lineage-specific engraftment of donor cells, e.g., the progeny of a progenitor cell, e.g., a transfected cell or a stem cell, e.g., a bone marrow-derived stem cell and/or a hematopoietic stem cell, e.g., lineage-specific cell detection. Current methods for measuring cell chimerism, e.g., hematopoietic cell chimerism, are based on DNA polymorphisms that distinguish recipient from donor. These methods provide an assessment of engraftment of nucleated donor cells and are, therefore, useful in determining the levels of leukocyte chimerism after transplant. However, these methods do not provide an assessment of the functional capacity of the engrafted cells, e.g., oxygen carrying erythrocytes or functional/reactive immune cells, e.g., functional engraftment, and do not directly examine engraftment of specific cell lineages following transplant. Moreover, these methods require prior purification of cellular subsets which often results in samples that are not representative of the original sample.

Similarly, gene therapy methods do not quantitate the functional capacity of donated cells or directly examine engraftment of specific cell lineages following transplant of transgenic cells. Current methods rely on the presence of the targeting vector and/or presence of functional protein which does not directly examine engraftment of specific cell lineages following transplant.

The present invention provides methods to accurately identify, detect, and quantify a functional cell and directly examine engraftment of specific cell lineages, following transplant, with out requiring prior purification of cellular subsets. In one embodiment, the present invention provides methods to identify, detect, and quantify the engraftment of donor cells that give rise to hematopoietic and non-hematopoietic lineages following stem cell transplantation. For example, the use of lineage-specific variants, such as, for example, allelic variants, e.g., single nucleotide polymorphisms (SNPs), allow for the identification and detection of lineage-specific cells, e.g., donor and recipient lineage-specific cells, to assess the functional engraftment of specific cell lineages, as well as the functional capacity of the engrafted cells.

In one embodiment, the methods of the invention utilize allelic variants, e.g., SNPs, contained within the coding regions of genes which are unique to specific cell lineages, e.g., erythroid, myeloid, or lymphoid lineages, in order to identify, detect, and quantify lineage specific chimerism, e.g., following transplantation, to thereby identify the functional outcome of the stem cell transplant or to determine the clinical outcome of a subject following progenitor cell transfer.

In one embodiment, the lineage-specific allelic variant, e.g. SNP, is a disease-related allelic variant, e.g., the sickle mutation in the β-globin gene. Because the β-globin gene is expressed only in erythroid lineage cells, the sickle mutation in the β-globin gene may be used to evaluate chimerism in the erythroid lineage, e.g., following stem cell transplantation, in a subject. RNA derived from the recipient may be distinguished from RNA derived from the donor based on the presence or absence of the sickle mutation.

In another embodiment, the lineage specific allelic variant, e.g., SNP, is a non-disease-related allelic variant. For example, the allelic variant may be any variant, e.g., SNP, provided that the allelic variant, e.g., SNP, is an expressed variant contained within a gene which is expressed only in a specific cell type or lineage, e.g., erythroid, myeloid, and lymphoid lineages. In one embodiment, the SNP is a high-frequency SNP. It is understood that in order to identify, detect, and quantify specific cell lineages following stem cell transplantation in a subject, the allelic variant must be polymorphic between the donor and the recipient, and the recipient genotype at the location of the variant must be ascertained prior to identifying, detecting, and quantifying the lineage-specific chimerism.

In still another embodiment, a panel of allelic variants, e.g., SNPs, is used to identify, detect, and quantify lineage-specific cells, e.g., in a high-throughput manner. In another embodiment, pyrosequencing is used to identify the presence or absence of allelic variants, e.g., SNPs, in a sample.

In a further embodiment, the methods of the invention are used to quantify immune reconstitution, e.g., reconstitution of immune cells, such as, for example, myeloid, T cell, B cell, monocyte, natural killer (NK) cell, and dendritic cells (DC), following stem cell transplantation, by identification of allelic variants, e.g., SNPs, contained within genes which are known to be unique to certain cell types, e.g., myeloid, T cell, B cell, monocyte, natural killer (NK) cell, and dendritic cells. Furthermore, various leukocyte populations can also be defined based on their functional activity. Therefore, in a further embodiment, the present invention provides methods for using allelic variants, e.g., SNPs present within functional molecules, to measure chimerism and determine whether effector activity of activated immune cell populations are host or donor derived. The presence of certain functional immune cell populations may be used to predict clinical outcome of a subject following transplantation. Examples of functional molecules include, for example, cytokines and secreted factors that are associated with specific cell subsets and markers associated with general T cell activation, T/NK cell cytolytic activity, Th1 and Th2 activity, DC activation and tolerance induction, chemokines and their receptors, and molecules associated with activation-induced signaling.

Definitions

For convenience, the meaning of certain additional terms and phrases employed in the specification, examples, and appended claims are provided below. Additional definitions are set forth throughout the detailed description.

As used herein, a “cell lineage” refers to cells with a common ancestry that develop from the same type of identifiable cell into specific identifiable/functioning cells. As used herein, a “progenitor cell”, e.g., a transfected cell or a stem cell, e.g., a bone marrow-derived stem cell and/or a hematopoietic stem cell, is a parent cell that gives rise to a distinct cell lineage by a series of cell divisions. Accordingly, a “lineage-specific cell” is intended to refer to any of the cells derived from a progenitor cell, e.g., a transfected cell or a stem cell, in the developmental series that ultimately produce identifiable and/or differentiated progeny cells. A lineage-specific cell may be identified based on a polymorphic region of a gene of interest.

As used herein, a “bone marrow-derived stem cell” is an undifferentiated, pluripotent cell that gives rise to, for example, adipocytes, cardiomyocytes, hepatocytes, osteoblasts, renal mesangial cells, endothelial cells, stromal cells, and/or chondrocytes. (see, for example, Asahara, T., et al. (1997) Science 275:964; Ito, T., et al. (2001) J Am Soc Nephrol 12:2625; Imasawa, T., et al. (2001) J Am Soc Nephrol 12:1401; Grant, M. B., et al. (2002) Nat Med 8:607; Grigoriadis, A. E., et al. (1988) J Cell Biol 106:2139; Lagasse, E., et al. (2000) Nat Med 6:1229; Makino, S., et al.(1999) J Clin Invest 103:697; Murohara, T. (2001) Trends Cardiovasc Med 11:303; Peichev, M., et al. (2000) Blood 95:952; Pittenger, M. F., et al. (1999) Science 284:143; Schatteman, G. C. and Awad, O. (2004) Anat Rec 276A:13).

As used herein, a “hematopoietic stem cell” is an undifferentiated, pluripotent cell that gives rise to blood cells, e.g., erythroid, myeloid and/or lymphoid cells, particularly highly specialized cells, which take the place of cells which die or are lost. Hematopoietic stem cells, e.g., embryonic stem cells, are unique in that they can both renew themselves, e.g., replicate, as well as create new cells, e.g., differentiate, throughout the life of the organism. Hematopoietic cells can differentiate into cells that are part of any tissue in an organism, e.g., blood cells, e.g., erythrocytes, leukocytes, granulocytes, monocytes and platelets, as well as cells comprising the fixed macrophage population, including Kupffer cells of the liver, pulmonary alveolar macrophages, osteoclasts, Langerhans cells of the skin and brain microglial cells.

A “lineage-specific erythroid cell” is intended to refer to any of the cells derived from hematopoietic stem cells in the developmental series that ultimately produce an erythrocyte. An “erythrocyte”, also referred to as a red blood cell, is a mature, functional, e.g., capable of transporting oxygen, non-nucleated, biconcave cell that contains hemoglobin, e.g., β-globin. Similarly, a “lineage-specific lymphoid cell” is intended to refer to any of the cells derived from hematopoietic stem cells in the developmental series that ultimately produce a lymphocyte, e.g., T cell or B cell, natural killer cell, dendritic cell, or plasma cell. As used herein, “lineage-specific myeloid cell” is intended to refer to any of the cells derived from hematopoietic stem cells in the developmental series that ultimately produce a granulocyte, monocyte or platelet.

As used herein, a “T cell” refers to T lymphocytes as defined in the art and is intended to include thymocytes, immature T lymphocytes, mature T lymphocytes, resting T lymphocytes, or activated T lymphocytes. The T cells can be CD4⁺ T cells, CD8⁺ T cells, CD4⁺CD8⁺ T cells, or CD4⁻CD8⁻ T cells. The T cells can also be T helper cells, such as T helper 1 (Th1) or T helper 2 (Th2) cells. The term T cells also include activated T cells and memory T cells.

As used herein, a “B cell” is cell expressing immunoglobulins on its cell surface. B cells mature into plasma cells which produce antibodies. B cells may also mature into memory B cells that produce the same antibody which is directed against the antigen that stimulated it to mature.

As used herein, the term “naive T cells” includes T cells that have not been exposed to cognate antigen and so are not activated and are not memory cells. Naive T cells are not cycling and human naive T cells are CD45RA+. If naive T cells recognize antigen and receive additional signals depending upon but not limited to the amount of antigen, route of administration and timing of administration, they may proliferate and differentiate into various subsets of T cells, e.g. effector T cells.

As used herein, the term “memory T cell” includes lymphocytes which, after exposure to antigen, become functionally quiescent and are capable of surviving for long periods in the absence of antigen. Human memory T cells are CD45RA−.

As used herein, the term “effector T cell” or “Teff cell” includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response (Mosmann and Coffinan, 1989, Annu. Rev. Immunol. 7, 145-173; Paul and Seder, 1994, Cell 76, 241-251; Arthur and Mason, 1986, J Exp. Med. 163, 774-786; Paliard, et al., 1988, J. Immunol. 141, 849-855; Finkelman, et al., 1988, J. Immunol. 141, 2335-2341).

As used herein, the term “T helper type 1 response” (Th1 response) refers to a response that is characterized by the production of one or more cytokines selected from IFN-γ, IL-2, TNF, and lymphotoxin (LT) and other cytokines produced preferentially or exclusively by Th1 cells rather than by Th2 cells. As used herein, a “T helper type 2 response” (Th2 response) refers to a response by CD4⁺ T cells that is characterized by the production of one or more cytokines selected from IL-4, IL-5, IL[-6 and IL-10, and that is associated with efficient B cell “help” provided by the Th2 cells (e.g., enhanced IgG1 and/or IgE production).

As used herein, the term “regulatory T cell” or “T reg cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4⁺CD25⁺ population of cells (see, e.g., Waldmann and Cobbold. 2001. Immunity. 14:399). Regulatory T cells actively suppress the proliferation and cytokine production of Th1, Th2, or naive T cells which have been stimulated in culture with an activating signal (e.g., antigen and antigen presenting cells or with a signal that mimics antigen in the context of MHC, e.g., anti-CD3 antibody, plus anti-CD28 antibody).

As used herein, the term “anergy” or “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2. Tolerance occurs when cells are exposed to antigen and receive a first signal (a T cell receptor or CD-3 mediated signal) in the absence of a second signal (a costimulatory signal) or by modulation, e.g., upmodulation of an inhibitory signal from an inhibitory receptor, such as, for example, ILT3. Under these conditions, reexposure of the cells to the same antigen (even if reexposure occurs in the presence of a costimulatory polypeptide) results in failure to produce cytokines and, thus, failure to proliferate. For example, tolerance is characterized by lack of cytokine production, e.g., IL-2, or can be assessed by use of a mixed lymphocyte culture assay. Tolerance can occur to self antigens or to foreign antigens.

As used herein, a “professional antigen presenting cell” or “APC” is a cell that can present antigen in a form that cells can recognize it. The cells that can “present” antigen include B cells, monocytes, macrophages and dendritic cells. As used herein, the term “dendritic cell” or “DC” is intended to include APCs capable of activating naive T cells and stimulating the growth and differentiation of B cells. DCs are lineage negative cells, i.e., they lack cell surface markers for T cells, B cells, NK cells, and monocytes/macrophages, however they strongly express various costimulatory molecules (e.g., CD86, CD80, CD83, and HLA-DR) and/or adhesion molecules.

As used herein, the term “immune response” includes T cell mediated and/or B cell mediated immune responses that are influenced by modulation of T cell costimulation. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells, e.g., macrophages.

As used herein, a “natural killer cell” or “NK cell” is a lymphocyte that originates in the bone marrow and can develop fully in the absence of the thymus. An NK cell recognizes and destroys foreign cells without prior sensitization to it.

A “transfected cell” is a lineage-specific cell that has been modified, e.g., stably transfected, in vivo or ex vivo, by genetic transfer techniques, e.g., gene targeting, e.g., gene therapy, to carry and replicate exogenous DNA, and whose progeny can be identified based on the genetic material they carry following transfection. “Gene therapy” is a process to manipulate the genome of an organism to prevent, mask, or lessen the effects of a genetic disease or disorder by the introduction of genetic material into the genome of targeted cells in order to correct a genetic defect or to add a new biologic property or function with therapeutic potential. This technique can be used to alter, for example, cellular metabolism, immune response, or sensitivity to therapeutic agents.

“Bone Marrow Transplantation” (BMT) also referred to as “blood-” or “marrow-derived stem cell transplantation”, “hematopoietic cell transplantation” (HCT), “hematopoietic stem cell transplantation” (HSCT) or “stem cell transplantation” (SCT), is a term used in the art to describe the collection and transplantation of progenitor cells, e.g., stem cells, e.g., bone marrow-derived stem cells and/or hematopoietic stem cells, from a donor and injection into a recipient. Following BMT, donor stem cells, e.g., bone marrow-derived stem cells and/or hematopoietic stem cells, home to the appropriate sites of the recipient and grow continuously along with remaining host and/or recipient cells and proliferate. By this process, newly formed and pre-existing cells (recipient), e.g., lymphoid and myeloid cells, are exposed to donor antigens and antibodies they produce so that the transplant may be recognized as self. The stem cells may be allogenic, e.g., stem cells from a donor who is not immunologically related to the recipient, or autologous, e.g., one's own stem cells. Stem cells, e.g., bone marrow-derived stem cells and/or hematopoietic stem cells, can be acquired from bone marrow, or alternatively from umbilical cord blood, peripheral blood or fetal liver or spleen.

As used herein, “hematopoietic chimerism” or “chimerism” is intended to describe the engraftment or survival of donor progenitor cells, e.g., stem cells, e.g., bone marrow-derived stem cells and/or hematopoietic stem cells, without continued immunosuppressive therapy, within a recipient organism, e.g., a mammal, e.g., a human. It is thought, although not intending to be bound by theory, that the mechanism that allows chimerism is a form of “immunological tolerance”, e.g. an immunologic response consisting of the development of specific non-reactivity of lymphoid tissues to a given antigen that in other circumstances induces cell-mediated or humoral immunity, e.g., graft versus host disease (GVHD).

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including, but not limited to, the genes listed in Tables 4, 6, 7, and 8, can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass fluctional allelic variants, non-functional allelic variants, SNPs, mutations and polymorphisms. Lineage-specific allelic variants are those variants, e.g., functional allelic variants, non-functional allelic variants, SNPs, mutations and polymorphisms, that distinguish one lineage-specific cell from another. For example, a lineage-specific allelic variant described herein and/or identified by the methods of the invention may be utilized to distinguish recipient progenitor cells e.g., transfected cells or stem cells, e.g., bone marrow-derived stem cells and/or hematopoietic stem cells, from donor progenitor cells e.g., transfected cells or stem cells, e.g., bone marrow-derived stem cells and/or hematopoietic stem cells. For example, an allelic variant of the β-globin gene is, for example, a form of the β-globin gene with the nucleotide T at position 70 of SEQ ID No: 1. Other allelic variants used in the methods of the present invention include, but are not limited to, those variants listed in Table 9 (see below) and identified by a GenBank accession number, i.e., the reference SNP (rs) number.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

“Amplifying” refers to template-dependent processes and/or vector-mediated propagation which results in an increase in the concentration of a specific nucleic acid molecule relative to its initial concentration, or to an increase in the concentration of a detectable signal. As used herein, the term template-dependent process is intended to refer to a process that involves the template-dependent extension of a primer molecule. The term “template-dependent process” refers to nucleic acid synthesis of an RNA, DNA or cDNA molecule wherein the sequence of the newly synthesized strand of nucleic acid is dictated by the well-known rules of complementary base pairing (see, for example, Watson, J. D. et al., In: Molecular Biology of the Gene, 4th Ed., W. A. Benjamin, Inc., Menlo Park, Calif. (1987)). Typically, vector mediated methodologies involve the introduction of the nucleic acid fragment into a DNA or RNA vector, the clonal amplification of the vector, and the recovery of the amplified nucleic acid fragment. Examples of such methodologies are provided by Cohen, et al. (U.S. Pat. No. 4,237,224), Maniatis, T. et al., Molecular Cloning (A Laboratory Manual), Cold Spring Harbor Laboratory, 1982).

“Biological activity” or “bioactivity” or “activity” or “biological function”, which are used interchangeably, for the purposes herein when applied to a gene of the invention, e.g., those genes corresponding to the molecules listed in Tables 4, 6, 7, 8, means an effector or antigenic function of the polypeptide encoded by the genes of the invention that is directly or indirectly performed by a polypeptide (whether in its native or denatured conformation), or by a fragment thereof. For example, biological activities of the β-globin polypeptide include transportation of oxygen and other biological activities, whether presently known or inherent. Additional bioactivities of the polypeptides encoded by the genes of the invention include but are not limited to, T cell activation, T cell proliferation, inflammation, cytolytic activity, helper T cell function, e.g., Th1 and Th2 cell function, DC activation, and tolerance induction. Bioactivity can be modulated by directly affecting a protein by, for example, changing the level of effector or substrate level. Alternatively bioactivity can be modulated by modulating the level of a protein, such as by modulating expression of a gene. Antigenic functions include possession of an epitope or antigenic site that is capable of cross-reacting with antibodies that bind a native or denatured polypeptide or fragments thereof.

Biologically active polypeptides include polypeptides having both an effector and antigenic function, or only one of such functions. For example, β-globin polypeptides include antagonist polypeptides and native β-globin polypeptides, provided that such antagonists include an epitope of a native β-globin polypeptide. An effector function of, for example, a β-globin polypeptide, can be the ability to bind to a ligand of a β-globin molecule.

As used herein the term “bioactive fragment of a protein” refers to a fragment of a full-length protein wherein the fragment specifically mimics or antagonizes the activity of a wild-type protein. The bioactive fragment preferably is a fragment capable of binding to a second molecule, such as a ligand.

The term “an aberrant activity” or “abnormal activity”, as applied to an activity of a protein refers to an activity which differs from the activity of the normal or reference protein or which differs from the activity of the protein in a healthy subject, e.g., a subject not afflicted with a disease or disorder as described herein. An activity of a protein can be aberrant because it is stronger than the activity of its wild-type counterpart. Alternatively, an activity of a protein can be aberrant because it is weaker or absent relative to the activity of its normal or reference counterpart. An aberrant activity can also be a change in reactivity. For example an aberrant protein can interact with a different protein or ligand relative to its normal or reference counterpart. A cell can also have aberrant activity due to overexpression or underexpression of a gene. For example, aberrant β-globin activity can result from a mutation in the gene, which results, e.g., in lower or higher binding affinity of a ligand to the β-globin protein encoded by the mutated gene. Aberrant β-globin activity can also result from an abnormal β-globin 5′ upstream regulatory element activity.

The phrase “biological sample” is intended to include solid and body fluid samples isolated from a subject, as well as those present within a subject. The biological samples used in the present invention can include cells, nucleic acids, protein or membrane extracts of cells, blood or biological fluids such as ascites fluid or brain fluid (e.g., cerebrospinal fluid). Examples of solid biological samples include, but are not limited to, samples taken from tissues of bone, e.g., bone marrow, the central nervous system, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Cells,” “host cells” or “recombinant host cells” are terms used interchangeably herein. It is understood that such terms refer not only to the particular cell but to the progeny or derivatives of such a cell. Because certain modifications may occur in succeeding generations due to either mutation or environmental influences, such progeny may not, in fact, be identical to the parent cell, but are still included within the scope of the term as used herein.

As used herein, the term “clinical course of therapy” refers to any chosen method to treat, prevent, or ameliorate a disease or disorder, e.g., a hematologic disease or disorder, e.g., sickle cell syndromes or thalassemia syndromes, symptoms thereof, or related diseases or disorders. Clinical courses of therapy include, but are not limited to, lifestyle changes (e.g., changes in diet or environment), administration of medication, e.g., immunosuppressive drugs, cellular therapy, such as lymphocyte infusion, use of medical devices, surgical procedures, cell transplantation, including stem cell transplantation, e.g., allogenic or autologous, e.g., myeloablative or nonmyeloablative, or any combination thereof.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the. molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The term “a homolog of a nucleic acid” refers to a nucleic acid having a nucleotide sequence having a certain degree of homology with the nucleotide sequence of the nucleic acid or complement thereof. For example, a homolog of a double stranded nucleic acid having SEQ ID No: 1 is intended to include nucleic acids having a nucleotide sequence which has a certain degree of homology with SEQ ID No:1 or with the complement thereof. Preferred homologs of nucleic acids are capable of hybridizing to the nucleic acid or complement thereof.

The term “hybridization probe” or “primer” as used herein is intended to include oligonucleotides which hybridize and/or bind in a base-specific manner to a complementary strand of a target nucleic acid. Such probes include peptide nucleic acids, and described in Nielsen et al., (1991) Science 254:1497-1500. Probes and primers can be any length suitable for specific hybridization to the target nucleic acid sequence. The most appropriate length of the probe and primer may vary depending on the hybridization method in which it is being used; for example, particular lengths may be more appropriate for use in microfabricated arrays, while other lengths may be more suitable for use in classical hybridization methods, and still others more appropriate for polymerase chain reactions. Such optimizations are known to the skilled artisan. Suitable probes and primers can range form about 5 nucleotides to about 30 nucleotides in length. For example, probes and primers can be 5, 6, 8, 10, 12, 14, 16, 18, 20, 22, 24, 25, 26,28 or 30 nucleotides in length. The probe or primer of the invention comprises a sequence that flanks and/or overlaps, at least one polymorphic site occupied by any of the possible variant nucleotides. The nucleotide sequence of an overlapping probe or primer can correspond to the coding sequence of the allele or to the complement of the coding sequence of the allele.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “molecular structure” of a gene or a portion thereof refers to the structure as defined by the nucleotide content (including deletions, substitutions, additions of one or more nucleotides), the nucleotide sequence, the state of methylation, and/or any other modification of-the gene or portion thereof.

The term “mutated gene” refers to an allelic form of a gene that differs from the predominant form in a population. A mutated gene is capable of altering the phenotype of a subject having the mutated gene relative to a subject having the predominant form of the gene. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), complementary DNA (cDNA), and, where appropriate, ribonucleic acid (RNA), e.g., mRNA. The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenine”, “cytidine”, “guanine”, and thymidine” and/or “A”, “C”, “G”, and “T”, respectively, are used. It is understood that if the nucleic acid is RNA, a nucleotide having a uracil base is uridine.

The term “complementary nucleotide sequence”, refers to the nucleotide sequence of the complementary strand of a nucleic acid strand having a specific sequence. “Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “oligonucleotide” is intended to include any single- or double stranded DNA or RNA. Oligonucleotides can be naturally occurring or synthetic, but are typically prepared by synthetic means. Preferred oligonucleotides of the invention include segments of the genes listed in Tables 4, 6, 7, and 8, or their complements. The segments can be between about 5 and about 250 bases, about 10-240, 20-220, 30-210, 40-200, 50-190, 70-170, 90-150, 100-140, or 110-130 bases. For example, the segments can be about 15-25 bases, 16, 17, 18, 19, 20, 21, 22, 23, 24, or 25 bases, preferably 21 bases.

The term “operably-linked” is intended to mean that the 5′ upstream regulatory element is associated with a nucleic acid in such a manner as to facilitate transcription of the nucleic acid from the 5′ upstream regulatory element.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurning most frequently in a selected population is often referred to as the reference and/or wildtype form. Other allelic forms are typically designated as alternative or variant alleles. Diploid organisms may be homozygous or heterozygous for allelic forms. A diallelic or biallelic polymorphism has two forms. A trialleleic polymorphism has three forms.

A “polymorphic gene” refers to a gene having at least one polymorphic region.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase to produce cDNA) in an appropriate buffer and at a suitable temperature. The length of a primer may vary but typically ranges from about 10, 15, 20, 25, 30, 35, 40, 45, 50, 55, 60, 70, 75, 80, 85, 90, to about 1000 nucleotides. A primer need not match the exact sequence of a template, but must be sufficiently complementary to hybridize with the template.

The term “primer pair” refers to a set of primers including an upstream primer that hybridizes with the 5′ end of the complement of the DNA sequence to be amplified and a downstream primer that hybridizes with the 3′ end of the sequence to be amplified.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a 5′ upstream regulatory sequence, including, but not limited to, a basic promoter, and include elements such as enhancers and silencers. The term “enhancer”, also referred to herein as “enhancer element”, is intended to include regulatory elements capable of increasing, stimulating, or enhancing transcription from a 5′ upstream regulatory element, including a basic promoter. The term “silencer”, also referred to herein as “silencer element” is intended to include regulatory elements capable of decreasing, inhibiting, or repressing transcription from a 5′ upstream regulatory element, including a basic promoter. Regulatory elements are typically present in 5′ flanking regions of genes. Regulatory elements also may be present in other regions of a gene, such as introns. Thus, for example, it is possible that a β-globin gene has regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of an operably linked DNA sequence preferentially in specific cells (e.g., cells of a specific tissue). Gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a protein, hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding Site Profile database.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of, for example, a gene corresponding to the molecules listed in Tables 4, 6, 7, and 8.

As used herein, the term “transfection” or “transfected with” refers to the introduction of exogenous nucleic acid into a mammalian cell and encompass a variety of techniques useful for introduction of nucleic acids into mammalian cells including. electroporation, calcium-phosphate precipitation, DEAE-dextran treatment, lipofection, microinjection and infection with viral vectors. Suitable methods for transfecting mammalian cells can be found in Sambrook et al. Molecular Cloning: A Laboratory Manual, 2nd Edition, Cold Spring Harbor Laboratory press (1989)) and other laboratory textbooks. Choice of suitable vectors for expression is well within the skill of the art. The nucleic acid is “in a form suitable for expression” in which the nucleic acid contains all of the coding and regulatory sequences required for transcription and translation of a gene, which may include promoters, enhancers and polyadenylation signals, and sequences necessary for transport of the molecule to the surface of the transfected cell, including N-terminal signal sequences. When the nucleic acid is a cDNA in a recombinant expression vector, the regulatory functions responsible for transcription and/or translation of the cDNA are often provided by viral sequences. Examples of commonly used viral promoters include those derived from polyoma, Adenovirus 2, cytomegalovirus and Simian Virus 40, and retroviral LTRs. Regulatory sequences linked to the cDNA can be selected to provide constitutive or inducible transcription, by, for example, use of an inducible promoter, such as the metallothienin promoter or a glucocorticoid-responsive promoter.

The term “transduction” is generally used herein when the transfection with a nucleic acid is by viral delivery of the nucleic acid. “Transformation” as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

One approach for introducing nucleic acid into cells is by use of a viral vector containing nucleic acid. Examples of viral vectors which can be used include retroviral vectors (Eglitis, M. A., et al. (1985) Science 230:1395; Danos, O. and Mulligan, R. (1988) Proc. Natl. Acad. Sci., USA 85:6460); Markowitz, D., et al. (1988) J. Virol. 6:1120), adenoviral vectors (Rosenfeld, M. A., et al. (1992) Cell 68:143) and adeno-associated viral vectors (Tratschin, J. D., et al. (1985) Mol. Cell. Biol. 5:3251). Infection of cells with a viral vector has the advantage that a large proportion of cells will receive nucleic acid, thereby obviating a need for selection of cells which have received nucleic acid, and molecules encoded within the viral vector, e.g. by a cDNA contained in the viral vector, are expressed efficiently in cells which have taken up viral vector nucleic acid.

Alternatively, nucleic acids can be expressed on a cell using a plasmid expression vector which contains nucleic acid. Suitable plasmid expression vectors include CDM8 (Seed, B. (1987) Nature 329:840) and pMT2PC (Kaufman, I. (1987) EMBO J. 6:187). Since only a small fraction of cells (about 1 out of 105) typically integrate transfected plasmid DNA into their genomes, it is advantageous to transfect a nucleic acid encoding a selectable marker into the tumor cell along with the nucleic acid(s) of interest. Preferred selectable markers include those which confer resistance to drugs such as G418, hygromycin and methotrexate. Selectable markers may be introduced on the same plasmid as the nucleic acid(s) of interest or may be introduced on a separate plasmid.

As used herein, the term “transgene” refers to a nucleic acid sequence which has been genetic-engineered into a cell. Daughter cells deriving from a cell in which a transgene has been introduced, e.g., a progenitor cell, are also said to contain the transgene (unless it has been deleted). A transgene can encode, e.g., a polypeptide, or an antisense transcript, partly or entirely heterologous, i.e., foreign, to the transgenic cell into which it is introduced, or, is homologous to an endogenous gene of the transgenic cell into which it is introduced, but which is designed to be inserted, or is inserted, into the organism's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

The term “treatment”, or “treating” as used herein, is defined as the application or administration of a therapeutic agent to a subject, implementation of lifestyle changes (e.g., changes in diet or environment), administration of medication, e.g., immunosuppressive agents, cellular therapy, such as lymphocyte infusion, use of medical devices, or, surgical procedures, cell transplantation, including stem cell transplantation e.g., allogenic or autologous, e.g., myeloablative or nonmyeloablative, application, administration of a therapeutic agent to an isolated cell or tissue or cell line from a subject, or any combination thereof, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. The term treatment or treating refers to either (I) the prevention of a disease or disorder (prophylaxis), or (2) the reduction or elimination of symptoms of the disease or disorder (therapy). The terms “prevention”, “prevent” or “preventing” as used herein refers to inhibiting, averting or obviating the onset or progression of a disease or disorder.

As used herein, the term “vector” refers to a nucleic acid molecule capable of transporting or replicating another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively-linked are referred to herein as “expression vectors”.

Isolated Nucleic Acid Molecules Used in the Methods of the Invention

The methods of the invention include the use of isolated nucleic acid molecules that encode for proteins that are useful for the identification of lineage-specific cells, including, but not limited to, those molecules listed in Tables 4, 6, 7, and 8, proteins, or biologically active portions thereof, as well as nucleic acid fragments sufficient for use as hybridization probes to identify nucleic acid molecules that encode for molecules that are useful for the identification of lineage-specific cells (e.g., RNA) and fragments for use, for example, as PCR primers for the amplification of nucleic acid molecules that encode for proteins that are useful for the identification of lineage-specific cells. It is understood that genes other than those listed in Tables 4, 6, 7, 8, and 9 may be used in the methods of the invention, including genes which are expressed only by a certain cell type, e.g., lineage-specific genes.

One example of a nucleic acid that is useful for the identification of lineage-specific cells is β-globin. The nucleotide sequence of the isolated human β-globin cDNA and the predicted amino acid sequence of the human β-globin polypeptide are shown in SEQ ID Nos: 1 and 2, respectively. The nucleotide sequence of β-globin is also described in GenBank Accession No. GI: 28302128 (SEQ ID No: 1) (the contents of which are included herein by reference). Other examples of nucleic acids that are useful for the identification of lineage-specific cells include, but are not limited to CD4 (SEQ ID Nos:25-26, GenBank Accession No. GI: 21314613), CD8 (SEQ ID Nos:27-28, GenBank Accession No. GI: 27886640; SEQ ID Nos:29-30, GenBank Accession No. GI: 27886641; SEQ ID Nos:31-32, GenBank Accession No. GI: 27886630; SEQ ID Nos:33-34, GenBank Accession No. GI: 27886638; SEQ ID Nos:35-36, GenBank Accession No. GI: 27886634; SEQ ID Nos:37-38, GenBank Accession No. GI: 27886636; SEQ ID Nos:39-40, GenBank Accession No. GI: 27886629; SEQ ID Nos:41-42, GenBank Accession No. GI: 27886632), KIR (SEQ ID Nos:43-44, GenBank Accession No. GI: 33589849; SEQ ID Nos:45-46, GenBank Accession No. GI: 31982877; SEQ ID Nos:47-48, GenBank Accession No. GI: 30102932; SEQ ID Nos:49-50, GenBank Accession No. GI: 23592225; SEQ ID Nos:51-52, GenBank Accession No. GI: 11968153; SEQ ID Nos:53-54, GenBank Accession No. GI: 7705567; SEQ ID Nos:55-56, GenBank Accession No. GI: 7657278; SEQ ID Nos:57-58, GenBank Accession No. GI: 7657276; SEQ ID Nos:59-60, GenBank Accession No. GI: 7657272; SEQ ID Nos:61-62, GenBank Accession No. GI: 7657270; SEQ ID Nos:63-64, GenBank Accession No. GI: 7019440; SEQ ID Nos:65-66, GenBank Accession No. GI: 6912475; SEQ ID Nos:67-68, GenBank Accession No. GI: 6912473; SEQ ID Nos:69-70, GenBank Accession No. GI: 6912471; SEQ ID Nos:71-72, GenBank Accession No. GI: 580305 1; SEQ ID Nos:73-74, GenBank Accession No. GI: 7657280); genes that encode proteins specific to the erythroid cell lineage, including but not limited to, Kell blood group antigen (SEQ ID Nos:80-81; GenBank Accession No. GI:17025233), Lutheran blood group antigen (SEQ ID Nos:82-83; GenBank Accession No. GI: 31543105), Glycophorin A (SEQ ID-Nos:84-85; GenBank Accession No. GI: 8051602), Glycophorin B (SEQ ID Nos:86-87; GenBank Accession No. GI: 8051603), Glycophorin C (SEQ ID Nos:88-89; variant 1; GenBank Accession No. GI: 21614502) (SEQ ID Nos:330-331; variant 2; GenBank Accession No. GI: 21614516), Rhesus blood group CcEe antigen (SEQ ID Nos:90-91; variant 1; GenBank Accession No. GI: 20336217) (SEQ ID Nos:332-333; variant 2; GenBank Accession No. GI: 20336222) (SEQ ID Nos:334-335; variant 3; GenBank Accession No. GI: 20336218), (SEQ ID Nos:336-337; variant 4; GenBank Accession No. GI: 20336220), Rhesus blood group B (SEQ ID Nos:92-93; GenBank Accession No. GI: 9966890), Solute carrier family 4, Diego blood group (SEQ ID Nos:94-95; GenBank Accession No. GI: 4507020), Solute, carrier family, Kidd blood group (SEQ ID Nos:96-97; GenBank Accession No. GI: 7706676), Alpha globin 1 (SEQ ID Nos:98-99; GenBank Accession No. GI: 14456711), Alpha globin 2 (SEQ ID Nos: 100-101; GenBank Accession No. GI: 14043068), Erythrocyte membrane protein band 4.2 (SEQ ID Nos:102-103; GenBank Accession No. GI: 4557558), Heme Oxygenase 1 (SEQ ID Nos:326-327; GenBank Accession No. GI: 4504436), Heme oxygenase 2 (SEQ ID Nos:328-329; GenBank Accession No. GI: 8051607); genes that encode proteins specific to the lymphocyte cell lineage, e.g., T cells, including but not limited to, CD3, (SEQ ID Nos:17-18, GenBank Accession No. GI: 4502668; SEQ ID Nos:19-20, GenBank Accession No. GI: 4502670; SEQ ID Nos:21-22, GenBank Accession No. GI: 4557428; SEQ ID Nos:23-24, GenBank Accession No. GI: 4557430), TCRA (SEQ ID Nos:104-105, GenBank Accession No. GI: 338765), LNK (SEQ ID Nos:106-107, GenBank Accession No. GI: 4885454), CD28 (SEQ ID Nos:108-109, GenBank Accession No. GI: 5453610); genes that encode proteins specific to the lymphocyte cell lineage, e.g., B cells, including but not limited to, CD20 (SEQ ID Nos:9-10, GenBank Accession No. GI: 23110988; SEQ ID Nos; 11-12, GenBank Accession No. GI: 23110990; SEQ ID Nos: 13-14, GenBank Accession No. GI: 23110986), CD19 (SEQ ID Nos:15-16, GenBank Accession No. GI: 32481214), CD22 (SEQ ID Nos: 110-111, GenBank Accession No. GI: 4502650), CD79A (SEQ ID Nos:112-113, variant 1, GenBank Accession No. GI: 4502684), (SEQ ID Nos:338-339, variant 2, GenBank Accession No. GI: 11038671), CD79B (SEQ ID Nos:114-115, variant 1, GenBank Accession No. GI: 11038673), (SEQ ID Nos:340-341, variant 2, GenBank Accession No. GI: 11038675), B cell linker (BLINK) (SEQ ID Nos: 116-117, GenBank Accession No. GI: 40353774); genes that encode proteins specific to the lymphocyte cell lineage, e.g., monocytes, including but not limited to, CD14 (SEQ ID Nos:118-119, GenBank Accession No. GI: 4557416); genes that encode proteins specific to the lymphocyte cell lineage, e.g., NK cells, including but not limited to, CD56 (NCAM1) (SEQ ID Nos:120-121, variant 1, GenBank Accession No. GI: 10834989), (SEQ ID Nos:342-343, variant 2, GenBank Accession No. GI: 41281936), CD94 (CLEC2) (SEQ ID Nos: 122-123, variant 1, GenBank Accession No. GI: 7669497), (SEQ ID Nos:344-345, variant 2, GenBank Accession No. GI: 7669498), CD16 (PCGR3A) (SEQ ID Nos: 124-125, GenBank Accession No. GI: 51593094), CD160 (SEQ ID Nos:126-127, GenBank Accession No. GI: 51702223); dendritic cell gene products, including, but not limited to, DC-SIGN (SEQ ID Nos:75-76; GenBank Accession No. GI: 22095359), DC-LAMP (SEQ ID Nos:128-129, GenBank Accession No. GI: 38455384), BDCA2 (SEQ ID Nos:130-131, variant 1, GenBank Accession No. GI: 45580689), (SEQ ID Nos:132-133, variant 2, GenBank Accession No. GI: 45580691), CD83 (SEQ ID Nos:134-135, GenBank Accession No. GI: 24475618); activated T cell gene products, including, but not limited to, IL2 (SEQ ID Nos:136-137, GenBank Accession No. GI: 28178860), CD69 (SEQ ID Nos:138-139, GenBank Accession No. GI: 4502680), IL7 (SEQ ID Nos:140-141, GenBank Accession No. GI: 28610152), IL15 (SEQ ID Nos:142-143, variant 3, GenBank Accession No. GI: 26787979) (SEQ ID Nos:346-347, variant 1, GenBank Accession No. GI:26787983) (SEQ ID Nos:348-349, variant 2, GenBank Accession No. GI: 26787985); proinflammatory cytokine, e.g., chemokine, gene products, including, but not limited to, IL1b (SEQ ID Nos:144-145, GenBank Accession No. GI: 27894305), TNFα (SEQ ID Nos:146-147, GenBank Accession No. GI: 25952110), IL6 (SEQ ID Nos: 148-149, GenBank Accession No. GI: 10834983), IL8 (SEQ ID Nos: 150-151, GenBank Accession No. GI: 28610153); gene products associated with NK cell and/or cytolytic T cell activity, including, but not limited to, Perforin (SEQ ID Nos:152-153, GenBank Accession No. GI: 45935369), Granzyme B (SEQ ID Nos:154-155, GenBank Accession No. GI: 32483414), Granulysin (SEQ ID Nos: 156-157, variant 1, GenBank Accession No. GI: 7108343) (SEQ ID Nos:350-351, variant 2, GenBank Accession No. GI: 7108345), IFNγ (SEQ ID Nos:158-159, GenBank Accession No. GI: 10835170); Th1 cell gene products, including, but not limited to, IFNγ (SEQ ID Nos:158-159, GenBank Accession No. GI: 10835170), TNFα (SEQ ID Nos:146-147, GenBank Accession No. GI: 25952110), TNFβ (SEQ ID Nos: 160-161, GenBank Accession No. GI: 6806892), GMCSF (SEQ ID Nos:162-163, GenBank Accession No. GI: 27437029); Th2 cell gene products, including, but not limited to, IL4 (SEQ ID Nos:164-165, variant 1, GenBank Accession No. GI: 27477090), (SEQ ID Nos:352-353, variant 2, GenBank Accession No. GI: 27477091), IL10 (SEQ ID Nos: 166-167, GenBank Accession No. GI: 24430216), IL13 (SEQ ID Nos:168-169, GenBank Accession No. GI: 26787977); gene products associated with DC activation, including, but not limited to, IL12A (SEQ ID Nos: 170-171, GenBank Accession No. GI: 24430218), IL12B (SEQ ID Nos:354-355, GenBank Accession No. GI: 24497437), IFNα (SEQ ID Nos:172-173, GenBank Accession No. GI: 13128949), IFNα5 (SEQ ID Nos:378-379, GenBank Accession No. GI: 4504596), IFNα13 (SEQ ID Nos:380-381, GenBank Accession No. GI: 13128965); gene products associated with tolerance induction, including, but not limited to, IL10 (SEQ ID Nos:166-167, GenBank Accession No. GI: 24430216), TGFβ (SEQ ID Nos:174-175, GenBank Accession No. GI: 10863872); genes that encode proteins specific to the endothelial cell lineage, including but not limited to, vascular cell adhesion molecule 1 isoform a (VCAM1) (SEQ ID Nos:176-177, variant 1, GenBank Accession No. GI: 18201907), (SEQ ID Nos:356-357, variant 2, GenBank Accession No. GI: 18201908), Nitric oxide synthase 3 (NOS3) (SEQ ID Nos:178-179, GenBank Accession No. GI: 48762674), von Willebrand factor precursor (VWF) (SEQ ID Nos:180-181, variant 1, GenBank Accession No. GI: 21265033), (SEQ ID Nos:358-359, variant 3, GenBank Accession No. GI: 21265042), (SEQ ID Nos:358-359, variant 3, GenBank Accession No. GI: 21265045), (SEQ ID Nos:360-361, variant 2, GenBank Accession No. GI: 21265045), (SEQ ID Nos:362-363, variant 4, GenBank Accession No. GI: 21265048), VE-Cadherin, (SEQ ID Nos:182-183, GenBank Accession No. GI: 14589894), VEGFRI (SEQ ID Nos:184-185, GenBank Accession No. GI: 32306519), VEGFR2 (SEQ ID Nos:186-187, GenBank Accession No. GI: 11321596), tie-2, an endothelial-specific tyrosine kinase (SEQ ID Nos:188-189, GenBank Accession No. GI: 4557868); genes that encode proteins specific to the stromal cell lineage, including but not limited to, fibronectin (SEQ ID Nos:190-191, variant 3, GenBank Accession No. GI: 47132558) (SEQ ID Nos:364-365, variant 7, GenBank Accession No. GI: 47132546), (SEQ ID Nos:366-367, variant 6, GenBank Accession No. GI: 47132548), (SEQ ID Nos:368-369, variant 2, GenBank Accession No. GI: 47132550), (SEQ ID Nos:370-371, variant 5, GenBank Accession No. GI: 47132552), (SEQ ID Nos:372-373, variant 1, GenBank Accession No. GI: 47132556), (SEQ ID Nos:374-375, variant 4, GenBank Accession No. GI: 47132554), vimentin (SEQ ID Nos:192-193, GenBank Accession No. GI: 4507894), smooth-muscle actin (SEQ ID Nos:194-195, GenBank Accession No. GI: 4501882), N-cadherin (SEQ ID Nos:196-197, GenBank Accession No. GI: 14589888); genes that encode proteins specific to the osteoblast cell lineage, including but not limited to, type I procollagen (SEQ ID Nos:198-199, GenBank Accession No. GI: 14719826), alkaline phosphatase (SEQ ID Nos:200-201, GenBank Accession No. GI: 13787192), osteocalcin (SEQ ID Nos:202-203, variant 1, GenBank Accession No. GI: 41152108), (SEQ ID Nos:376-377, variant 2, GenBank Accession No. GI: 41152108), (all the contents of which are included herein by reference).

The methods of the invention include the use of isolated nucleic acid molecules that encode proteins or biologically active portions thereof of, for example, the molecules listed in Tables 4, 6, 7, and 8, as well as nucleic acid fragments sufficient for use as hybridization probes to identify nucleic acid molecules encoding, e.g., the molecules listed in Tables 4, 6, 7, and 8, and fragments for use, for example, as PCR primers for the amplification of nucleic acid molecules, e.g., the molecules listed in Tables 4, 6, 7, and 8. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, or a portion thereof, can be isolated using standard molecular biology techniques and the sequence information provided herein. Using all or portion of the nucleic acid sequence of SEQ ID No: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, as hybridization probes, nucleic acid molecules corresponding to the molecules listed in Tables 4, 6, 7, and 8, can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, can be isolated by the polymerase chain reaction (PCR), including RT-PCR, using synthetic primers and/or primer pairs designed based upon the sequence of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380.

A nucleic acid used in the methods of the invention can be amplified using cDNA, RNA or, alternatively, genomic DNA as a template and appropriate primers and/or primer pairs according to standard PCR amplification techniques. Furthermore, primers and/or primer pairs corresponding to the nucleotide sequences of, e.g., the molecules listed in Tables 4, 6, 7, and 8, can be prepared by standard synthetic techniques, e.g., using an automated DNA synthesizer. The primers and/or primer pairs of the invention may further comprise a label group attached thereto e.g., biotin, and in addition to their use in PCR and RT-PCR may be utilized in sequencing reactions, e.g., pyrosequencing, or CHIP-based or array-based detection methods, e.g., using Affymetrix Gene CHIP systems. Particularly preferred oligonucleotides for use in the methods of the invention include SEQ ID Nos. 3-8.

The design of additional oligonucleotides, primers and/or primer pairs for use in the amplification of isolated nucleic acid molecules of, e.g. the molecules listed in Tables 4, 6, 7, and 8, and/or detection of allelic variants (discussed below) of, e.g., the molecules listed in Tables 4, 6, 7, and 8, by the methods of the invention is within the scope of the invention. Suitable oligonucleotides, primers and/or primer pairs for the detection of allelic variants, for use in PCR and/or for sequence analysis of the genes of the invention, e.g., the molecules listed in Tables 4, 6, 7, and 8, can be readily designed using this sequence information and standard techniques known in the art for the design and optimization of oligonucleotide, primer and/or primer pair sequences. Optimal design of such oligonucleotides, primers and/or primer pairs sequences is achieved, for example, by the use of commercially available primer selection programs such as Primer 2.1, Primer 3 or GeneFisher.

In a preferred embodiment, the isolated nucleic acid molecules used in the methods of the invention comprise the nucleotide sequences shown in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, a complement of the nucleotide sequence shown in SEQ ID Nos: 1,9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, or a portion of any of these nucleotide sequences. A nucleic acid molecule which is complementary to the nucleotide sequences shown in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67,69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380 is one which is sufficiently complementary to the nucleotide sequences shown in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, such that it can hybridize to the nucleotide sequence shown in SEQ ID No: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequences shown in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, or a portion of any of these nucleotide sequences.

Moreover, the nucleic acid molecules used in the methods of the invention can comprise only a portion of the nucleic acid sequences of SEQ ID No: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, for example, a fragment which can be used as a probe or primer or a fragment encoding, for example, a portion of molecule listed in Tables 4, 6, 7, and 8, that contains an allelic variant and/or mutation. The probe and/or primer typically comprise substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, of an anti-sense sequence of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, or of a naturally occurring allelic variant or mutant of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380. In one embodiment, a nucleic acid molecule used in the methods of the present invention comprises a nucleotide sequence which is greater than 100, 100-200, 200-300, 300-400, 400-500, 500-600, 600-700, or more nucleotides in length and hybridizes under stringent hybridization conditions to a nucleic acid molecule of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel, et al., eds., John Wiley & Sons, Inc. (1995), sections 2, 4 and 6. Additional stringent conditions can be found in Molecular Cloning: a Laboratory Manual, Sambrook, et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀ [Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C, see e.g., Church and Gilbert (1984) Proc. Natl. Acad. Sci., USA 81:1991-1995, or alternatively 0.2×SSC, 1% SDS.

The methods of the invention further encompass the use of nucleic acid molecules that differ from the nucleotide sequences shown in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112,114, 116, 118, 120, 122,124, 126, 128, 130, 132, 134, 136, 138, 140, 142, 144, 146, 148, 150, 152, 154, 156, 158, 160, 162,.164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, due to degeneracy of the genetic code and thus encode the same proteins (corresponding to the molecules listed in Tables 4, 6, 7, and 8) as those encoded by the nucleotide sequences shown in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380. In another embodiment, an isolated nucleic acid molecule included in the methods of the invention has a nucleotide sequence encoding a protein having an amino acid sequence shown in SEQ ID Nos:2, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 331, 333, 335, 337, 339, 341, 343, 345, 347, 349, 351, 353, 355, 357, 359, 361, 363, 365, 367, 369, 371, 373, 375, 379, and 381.

The methods of the invention further include the use of and/or identification of lineage-specific allelic variants, e.g., functional allelic variants, non-functional allelic variants, SNPs, mutations and polymorphisms, of, e.g., the molecules listed in Tables 4, 6, 7, and 8. Functional allelic variants are naturally occurring amino acid sequence variants of the proteins of the invention, e.g., the protein corresponding to the molecules listed in Tables 4, 6, 7, and 8, that maintain an activity of the molecules listed in Tables 4, 6, 7, and 8. Functional allelic variants will typically contain only conservative substitution of one or more amino acids of SEQ ID No:2, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 201, 203, 331, 333, 335, 337, 339, 341, 343, 345, 347, 349, 351, 353, 355, 357, 359, 361, 363, 365, 367, 369, 371, 373, 375, 379, and 381, or substitution, deletion or insertion of non-critical residues in non-critical regions of the protein. Non-functional allelic variants are naturally occurring amino acid sequence variants of the human proteins corresponding to the molecules listed in Tables 4, 6, 7, and 8 that do not have an activity of the molecules listed in Tables 4, 6, 7, and 8. Non-functional allelic variants will typically contain a non-conservative substitution, deletion, or insertion or premature truncation of the amino acid sequence of SEQ ID No:2, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 201, 203, 331, 333, 335, 337, 339, 341, 343, 345, 347, 349, 351, 353, 355, 357, 359, 361, 363, 365, 367, 369, 371, 373, 375, 379, and 381, or a substitution, insertion or deletion in critical residues or critical regions of the protein.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in, for example, a β-globin protein, is preferably replaced with another amino acid residue from the same side chain family. In similar fashion, the amino acid repertoire can be grouped as acidic (e.g., aspartate, glutamate); basic (e.g., lysine, arginine histidine), aliphatic (e.g., glycine, alanine, valine, leucine, isoleucine, serine, threonine, with serine and threonine optionally be grouped separately as aliphatic-hydroxyl); aromatic (e.g., phenylalanine, tyrosine, tryptophan); amide (e.g., asparagine, glutamine); and sulfur-containing (e.g., cysteine and methionine). (see, for example, Biochemistry, 2^{nd} ed., Ed. by L. Stryer, W H Freeman and Co.: 1981). Whether a change in the amino acid sequence of a peptide results in a functional homolog, e.g. a functional β-globin homolog, (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Multiple allelic variants of the genes of the invention, e.g. genes corresponding to the molecules listed in Tables 4, 6, 7, and 8, have been identified and can be referenced by one of skill in the art. For example, allelic variants of β-globin that cause sickle cell syndromes have been identified and include, but are not limited to, a Glutamic acid to Valine substitution at amino acid 7 of SEQ ID No. :1 (referred to as Hb Sickle or HbS), a Glutamic acid to Lysine substitution at amino acid 7 of SEQ ID No.:1 (referred to as HbC), a Glutamic acid to Valine substitution at amino acid 27 of SEQ ID No.:1 (referred to as HbE), a Valine to Methionine substitution at amino acid 99 of SEQ ID No.:1 (referred to as Hb Koln), an Aspartate to Histidine substitution at amino acid 100 of SEQ ID No.:1 (referred to as Hb Yakima), an Asparagine to Lysine substitution at amino acid 103 of SEQ ID No.:1 (referred to as Hb Kansas), and a Histidine to Tyrosine substitution at amino acid 88 of SEQ ID No.:1 (referred to as Hb M. Iwata). Causative allelic variants of thalassemia syndromes have also been identified and number more than 125. See, e.g., Schwartz E, Benz E J, Forget B G. Thalassemia Syndromes. Hematology: Basic Principles and Practice. 1995:586-610, incorporated herein by reference. Additional allelic variants of the molecules of the invention are listed in Table 9 based on their GeneBank accession number.

Numerous procedures for determining the nucleotide sequence of a nucleic acid molecule, or for determining and/or detecting the presence of lineage-specific allelic variants, e.g., functional allelic variants, non-functional allelic variants, SNPs, mutations, and polymorphisms, in nucleic acid molecules, often include a nucleic acid amplification step, which can be carried out by, e.g., polymerase chain reaction (PCR).

Accordingly, in one embodiment, the invention provides primers for amplifying portions of a gene, e.g., a gene corresponding to the molecules listed in Tables 4, 6, 7, and 8, such as portions of exons and/or portions of introns. In a preferred embodiment, the exons and/or sequences adjacent to the exons of the human gene, e.g., the genes corresponding to the molecules listed in Tables 4, 6, 7, and 8, will be amplified to, e.g., detect which allelic variant, e.g., lineage-specific allelic variant, if any, of a polymorphic region is present in the gene, e.g., the genes corresponding to the molecules listed in Tables 4, 6, 7, and 8, of a subject. Preferred primers comprise a nucleotide sequence complementary a lineage-specific specific allelic variant of a polymorphic region, e.g., a polymorphic region of a gene corresponding to the molecules listed in Tables 4, 6, 7, and 8, and of sufficient length to selectively hybridize with a gene, e.g., a gene corresponding to the molecules listed in Tables 4, 6, 7, and 8, or a combination thereof. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a gene, e.g., gene corresponding to the molecules listed in Tables 4, 6, 7, and 8.

In an even more preferred embodiment, the primer is capable of hybridizing to a nucleotide sequence, e.g., a nucleotide sequence of a molecule listed in Tables 4, 6, 7, and 8, complements thereof, lineage-specific allelic variants thereof, or complements of allelic variants thereof. For example, primers comprising a nucleotide sequence of at least about 15 consecutive nucleotides, at least about 25 nucleotides or having from about 15 to about 20 nucleotides as set forth, for example, in SEQ ID NOs:3-8, or the complement thereof, and are provided by the invention. Primers having a sequence of more than about 25 nucleotides are also within the scope of the invention.

A primer or probe can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of a gene, e.g., a gene corresponding to a molecule listed in Tables 4, 6, 7, and 8, and which by hybridization or absence of hybridization to the DNA of a subject or the type of hybrid formed will be indicative of the identity of the allelic variant of the polymorphic region of the gene, e.g., a gene corresponding to a molecule listed in Tables 4, 6, 7, and 8.

Primers can be complementary to nucleotide sequences located close to each other or further apart, depending on the use of the amplified DNA. For example, primers-can be chosen such that they amplify DNA fragments of at least about 10 nucleotides or as much as several kilobases. Preferably, the primers of the invention will hybridize selectively to nucleotide sequences, e.g., nucleotide sequences corresponding to the molecules listed in Tables 4, 6, 7, and 8, located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to an allelic variant of a polymorphic region of a gene, e.g., a gene corresponding to a molecule listed in Tables 4, 6, 7, and 8. Thus, such primers can be specific for a gene sequence, e.g., a gene sequence corresponding to a molecule listed in Tables 4, 6, 7, and 8, so long as they have a nucleotide sequence which is capable of hybridizing to a gene, e.g., a gene corresponding to a molecule listed in Tables 4, 6, 7, and 8. Such primers can be used, e.g., in sequence specific oligonucleotide priming as described further herein.

Other preferred primers used in the methods of the invention are nucleic acids which are capable of hybridizing to the reference sequence of a gene, e.g., a gene corresponding to a molecule listed in Tables 4, 6, 7, and 8, thereby detecting the presence of the reference allele of an allelic variant or the absence of a variant allele of an allelic variant in a gene, e.g., a gene corresponding to a molecule listed in Tables 4, 6, 7, and 8. Such primers can be used in combination, e.g., with primers specific for the variant polynucleotide of the gene, e.g., the gene corresponding to a molecule listed in Tables 4, 6, 7, and 8. The sequences of primers specific for the reference sequences comprising the gene, e.g., a gene corresponding to a molecule listed in Tables 4, 6, 7, and 8, will be readily apparent to one of skill in the art.

The nucleic acids of the invention, e.g., the nucleic acids corresponding to a molecule listed in Tables 4, 6, 7, and 8, can also be used as probes, e.g. in therapeutic and diagnostic assays. For instance, the present invention provides a probe comprising a substantially purified oligonucleotide, which oligonucleotide comprises a region having a nucleotide sequence that is capable of hybridizing specifically to a region of a gene, e.g., a gene corresponding to a molecule listed in Tables 4, 6, 7, and 8, which is polymorphic. In an even more preferred embodiment of the invention, the probes are capable of hybridizing specifically to one allelic variant of a gene, e.g. a gene corresponding to a molecule listed in Tables 4,. 6, 7, and 8, having a nucleotide sequence which differs from the nucleotide sequence set forth in SEQ ID Nos:1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380. Such probes can then be used to specifically detect which allelic variant of a polymorphic region of a gene, e.g., a gene corresponding to a molecule listed in Tables 4, 6, 7, and 8, is present in a subject. The polymorphic region can be located in the 3′ UTR, 5′ upstream regulatory element, exon, or intron sequences of a gene, e.g., a gene corresponding to a molecule listed in Tables 4, 6, 7, and 8.

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a gene corresponding to a molecule listed in Tables 4, 6, 7, and 8 may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, so as to be more stable than naturally occurring nucleotides. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988), hybridization-triggered cleavage agents. (See, e.g., Krol et al., 1988, BioTechniques 6:958-976) or intercalating agents (see, e.g., Zon, 1988, Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising an intronic sequence, e.g., an intronic sequence corresponding to a molecule listed in Tables 4, 6, 7, and 8, may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The invention also provides vectors and plasmids comprising the nucleic acids of the invention. For example, in one embodiment, the invention provides a vector comprising at least a portion of a gene comprising a polymorphic region, e.g., a polymorphic region of a gene corresponding to a molecules listed in Tables 4, 6, 7, and 8. Thus, the invention provides vectors for expressing at least a portion of the allelic variants of the human gene reference sequences, e.g., the gene reference sequences corresponding to a molecule listed in Tables 4, 6, 7, and 8, as well as other allelic variants, comprising a nucleotide sequence which is different from the nucleotide sequences disclosed in, GI: 28302128, GI: 21314613, GI: 27886640, GI: 27886641, GI: 27886630, GI: 27886638, GI: 27886634, GI: 27886636, GI: 27886629, GI: 27886632, GI: 33589849, GI: 31982877, GI: 30102932, GI: 23592225, GI:11968153, GI: 7705567, GI: 7657278, GI: 7657276, GI: 7657272, GI: 7657270, GI: 7019440, GI: 6912475, GI: 6912473, GI: 6912471, GI: 5803051, GI: 7657280, GI:17025233, GI: 31543105, GI: 8051602, GI: 8051603, GI: 21614502, GI: 21614516, GI: 20336217, GI: 20336222, GI: 20336218, GI: 20336220, GI: 9966890,GI: 4507020, GI: 7706676, GI: 14456711,GI:14043068, GI:4557558,GI: 4504436,GI:8051607, GI:4502668; GI: 4502670, GI: 4557428, GI: 4557430, GI: 338765, GI: 4885454, GI: 5453610, GI: 23110988, GI: 23110990, GI: 23110986, GI: 32481214, GI: 4502650, GI: 4502684, GI: 11038671,GI:11038673, GI:11038675, GI:40353774,GI:4557416, GI:10834989, GI: 41281936, GI: 7669497, GI: 7669498, GI: 51593094, GI: 51702223, GI: 22095359, GI: 38455384, GI: 45580689, GI: 45580691, GI: 24475618, GI: 28178860, GI: 4502680, GI: 28610152, GI: 26787979, GI:26787983, GI: 26787985, GI: 27894305, GI: 25952110, GI:10834983, GI: 28610153, GI: 45935369, GI: 32483414, GI: 7108343,GI: 7108345,GI:10835170,GI: 10835170,GI:25952110, GI: 6806892, GI: 27437029, GI: 27477090, GI: 27477091, GI: 24430216, GI: 26787977, GI: 24430218, GI: 24497437, GI:13128949, GI: 24430216, GI:10863872, GI: 18201907, GI: 18201908, GI: 48762674, GI: 21265033, GI: 21265042, GI: 21265045, GI: 21265045, GI: 21265048, GI: 14589894, GI: 32306519, GI: 11321596, GI: 4557868, GI: 47132558, GI: 47132546, GI: 47132548, GI: 47132550, GI: 47132552, GI: 47132556, GI: 47132554, GI: 4507894, GI: 4501882, GI: 14589888, GI: 14719826, GI: 13787192, GI: 41152108, GI: 41152108, GI: 4504596, and GI: 13128965. The allelic variants can be expressed in eukaryotic cells, e.g., cells of a subject, e.g., a mammalian subject, or in prokaryotic cells.

The nucleic acid molecules of the present invention include lineage-specific allelic variants of the, e.g., genes corresponding to a molecule listed in Tables 4, 6, 7, and 8, which differ from the reference sequences set forth in SEQ ID Nos:1, 9, 11, 13, 15, 17, 19, 21, 23, 25, 27, 29, 31, 33, 35, 37, 39, 41,43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, or at least a portion thereof, having a polymorphic region. The preferred nucleic acid molecules of the present invention comprise β-globin sequence having the polymorphisms identified in Table 1 and those listed in Table 9. The invention further comprises isolated nucleic acid molecules complementary to nucleic acid molecules comprising the polymorphisms of the present invention. Nucleic acid molecules of the present invention can function as probes or primers, e.g., in methods for determining the allelic identity of a, e.g., polymorphic region corresponding to a molecule listed in Tables 4, 6, 7, and 8. The nucleic acids of the invention can also be used, either in combination with each other or in combination with other SNPs in the, e.g., genes corresponding to a molecule listed in Tables 4, 6, 7, and 8, or other genes to detect lineage-specific cells or monitor the effectiveness of treatment in a subject. The nucleic acids of the invention can further be used to prepare or express, e.g., genes, polypeptides encoded by specific alleles, such as mutant alleles corresponding to a molecule listed in Tables 4, 6, 7, and 8. Polypeptides encoded by specific alleles, such as mutant alleles corresponding to a molecule listed in Tables 4, 6, 7, and 8, polypeptides, can also be used in therapy or for preparing reagents, e.g., antibodies, for detecting proteins corresponding to a molecule listed in Tables 4, 6, 7, and 8, and proteins encoded by these alleles. Accordingly, such reagents can be used to detect mutant proteins, e.g., mutant proteins corresponding to a molecule listed in Tables 4, 6, 7, and 8.

The invention also provides isolated nucleic acids comprising at least one polymorphic region of a gene, e.g., a gene corresponding to a molecule listed in Tables 4, 6, 7, and 8, having a nucleotide sequence which differs from the reference nucleotide sequences set forth in SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380. Preferred nucleic acids can have a polymorphic region in an upstream regulatory element, an exon, an intron, or in the 3′ UTR.

Methods of the Invention

The invention provides methods for detecting lineage-specific cells by identifying lineage-specific mRNA. In one embodiment of the invention, a method is provided to detect lineage-specific cells in a biological sample including the steps of (a) isolating mRNA from the biological sample; (b) reverse transcribing cDNA from the mRNA; (c) amplifying cDNA; and (d) identifying lineage-specific cDNA in the biological sample. In certain embodiments of the invention, the identification of lineage-specific cells can be used to evaluate various clinical factors, such as ABO incompatibility, and the composition and intensity of the conditioning regimen on functional engraftment of lineage-specific cells.

In another aspect of the invention, a method is provided to detect lineage-specific cells in a biological sample including the steps of (a) isolating mRNA from the biological sample; (b) reverse transcribing cDNA from the mRNA; (c) amplifying said at least one allelic variant from the cDNA; and (d) identifying at least one lineage-specific allelic variant, e.g., SNP, in the sample. The method may be used to detect donor-derived lineage-specific cells or recipient-derived lineage-specific cells, or both.

Another aspect of the invention provides a method of quantifying progenitor cell transfer in a subject comprising the steps of (a) obtaining a biological sample prior to said progenitor cell transfer; (b) obtaining a biological sample following said progenitor cell transfer; (c) identifying and quantifying at least one lineage-specific allelic variant in said biological sample obtained in step (a); (d) identifying and quantifying at least one lineage-specific allelic variant in said biological sample obtained in step (b); and (e) comparing the quantity of donor-derived cells and recipient-derived cells in the samples, thereby quantifying progenitor cell transfer in a subject.

In another aspect of the invention, a method is provided to determine an effective dose of progenitor cell transfer in a subject comprising the steps of; (a) obtaining a biological sample prior to said progenitor cell transfer; (b) obtaining a biological sample following said progenitor cell transfer; (c) identifying and quantifying at least one lineage-specific allelic variant in said biological sample obtained in step (a), thereby quantifying progenitor cell transfer in said biological sample; (d)) identifying and quantifying at least one lineage-specific allelic variant in said biological sample obtained in step (b), thereby quantifying progenitor cell transfer in said biological sample; and (e) comparing the quantity of progenitor cell transfer from step (c) and step (d) to therapy outcome, thereby determining an effective dose of progenitor cell transfer.

In still other embodiments of the invention the methods are useful for monitoring functional engraftment of lineage-specific cells following progenitor cell transfer in a subject suffering from a disease or disorder, for monitoring immune reconstitution following progenitor cell transfer in a subject, and for determining the clinical outcome of a progenitor cell transfer in a subject. In one embodiment, the presence of at least one donor-derived lineage-specific allelic variant selected from the group listed in Table 7 is an indication of poor outcome, e.g., graft rejection, graft versus host disease, pulmonary hypertension, development of proteinuria and development of progressive renal insufficiency, and/or end-organ failure.

In another embodiment, the presence of at least one recipient-derived lineage-specific allelic variant selected from the group listed in Table 7 is an indication of favorable outcome, e.g., graft acceptance.

In another embodiment of the invention, two or more lineage-specific alleleic variants are detected and/or identified.

Thus, the invention relates to the identification, quantification, and/or detection of lineage-specific allelic variants. Numerous methods are available that can be utilized for both identifying lineage-specific allelic variants and detecting lineage-specific allelic variants. For example, SNPs in a gene of interest, e.g., a gene corresponding to a molecule listed in Tables 4, 6, 7, and 8, can be identified by searching various databases that compile and list SNPs identified through the sequencing efforts of the Human Genome Project. These databases include, but are not limited to, the SNP Consortium, (see, for example, Thorisson G A and Stein L D. (2003) Nucleic Acids Res. 31(1):124-7), SNPper, available through The Innate Immunity Programs for Genomic Applications (IIPGA), which is a collaboration between the Respiratory Sciences Center at the University of Arizona, The Respiratory and Genetics Research Group at the Channing Laboratory at Brigham and Women's Hospital and The Boston Children's Hospital Informatics Program (CHIP), NCBI SNP database (see, for example, Sherry, ST, et al. (1999) Genome Res. 9(8):677-9), the NCI Genetic Annotation Initiative (see, for example, Clifford R., et al. (2000) Genome Res. 10:1259-65), the Whitehead SNP database (see, for example, Wang, D G, et al. (1998) Science 280(5366):1077-82), Jsnps (see, for example, Hirakawa, M., et al. (2002) Nuc. Acids Res. 30(1):158-162), Genesnps, available through the University of Utah Genome Center, Polyphen (see, for example, Sunyaev, S., et al. (2001) Hum Mol Genet 10:591-597), OMIM (see, for example, McKusick, V. A.(1998) Mendelian Inheritance in Man. Catalogs of Human Genes and Genetic Disorders. Baltimore: Johns Hopkins University Press, (12th edition), HGBASE (see, for example, Fredman, D., et al. (2002) Nuc. Acids Res. 30:387-91), Celera (see, for example, Venter, J. C., et al. (2001) Science 291(5507):1304-51), Incyte Genomics Lifeseq database, or any combination thereof.

The present invention provides methods for identifying lineage-specific allelic variants by determining the molecular structure of a gene, such as a human gene corresponding to a molecule listed in Tables 4, 6, 7, and 8, or a portion thereof. In one embodiment, determining the molecular structure of at least a portion of a gene comprises determining the identity of the lineage-specific allelic variant of at least one polymorphic region of a gene, e.g., a gene corresponding to a molecule listed in Tables 4, 6, 7, and 8 (determining the presence or absence of a lineage-specific allelic variant of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, and 374, or the complement thereof). A polymorphic region of a lineage-specific gene can be located in an exon, an intron, at an intron/exon border, or in the 5′ upstream regulatory element of the gene, e.g., a gene corresponding to a molecule listed in Tables 4, 6, 7, and 8.

In one embodiment, the methods of the invention can be used to identify the presence or absence of a specific lineage-specific allelic variant of one or more polymorphic regions of a gene in a biological sample. The allelic differences can be: (i) a difference in the identity of at least one nucleotide or (ii) a difference in the number of nucleotides, which difference can be a single nucleotide or several nucleotides. The invention also provides methods for detecting differences in a gene such as chromosomal rearrangements, e.g., chromosomal dislocation.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allehic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g. in Cronin et al. (1996) Human Mutation 7:244. In one embodiment, a chip comprises all the allelic variants of at least one polymorphic region of a gene. In another embodiment, a chip comprises one of the allelic variants of at least one polymorphic region of a gene. In still another embodiment, the chip comprises one or more of the allelic variants of at least one polymorphic region of a gene. In one embodiment, the chip comprises a panel of allelic variants of at least one polymorphic region of a gene, such as for example, those variants listed in one or more of the Tables 1 and 9. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In certain aspects of the methods of the invention, it is necessary to first amplify at least a portion of a gene prior to identifying and/or detecting a lineage-specific allelic variant. Amplification can be performed, e.g., by PCR and/or LCR (see Wu and Wallace, (1989) Genomics 4:560), according to methods known in the art. In one embodiment, DNA and/or RNA of a cell is exposed to two PCR primers and amplification for a number of cycles sufficient to produce the required amount of amplified DNA and/or cDNA. In preferred embodiments, the primers are located between 100 and 350 base pairs apart.

In some cases, the presence of a lineage-specific allele of a gene in DNA from a subject can be shown by restriction enzyme analysis. For example, a specific nucleotide polymorphism can result in a nucleotide sequence comprising a restriction site which is absent from the nucleotide sequence of another lineage-specific allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g., RNA or DNA, obtained from a tissue sample. The double-stranded duplexes are treated with an agent which cleaves single-stranded regions of the duplex such as duplexes formed based on base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl. Acad. Sci., USA 85:4397; Saleeba, et al. (1992) Methods Enzymol. 217:286-295. In a preferred embodiment, the control or sample nucleic acid is labeled for detection.

In another embodiment, a lineage-specific allelic variant can be identified and/or detected by denaturing high-performance liquid chromatography (DHPLC) (Oefner and Underhill, (1995) Am. J Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan, et al. (1998) Genomics 52:44-49).

In other embodiments, alterations in electrophoretic mobility are used to identify and/or detect the type of lineage-specific allelic variant. For example, single strand conformation polymorphism (SSCP) may be used to detect differences in electrophoretic mobility between mutant and wild type nucleic acids (Orita et al. (1989) Proc. Natl. Acad. Sci., USA 86:2766; see also Cotton (1993) Mutat Res. 285:125-144; and Hayashi (1992) Genet. Anal. Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen, et al. (1991) Trends Genet. 7:5).

In yet another embodiment, the identity of a lineage-specific allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers, et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Other examples of techniques for identifying and/or detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then-hybridized to target DNA under conditions which permit hybridization only if a perfect match is found (Saiki, et al. (1986) Nature 324:163); Saiki, et al. (1989) Proc. Natl. Acad. Sci., USA 86:6230; and Wallace, et al. (1979) Nucl. Acids Res. 6:3543). Such lineage-specific allele specific oligonucleotide hybridization techniques may be used for the simultaneous detection of several nucleotide changes in different polymorphic regions of a gene of interest. For example, oligonucleotides having nucleotide sequences of specific lineage-specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs, et al. (1989) Nucleic Acids Res. 17:2437-2448) or at the extreme 3′ end of one primer where, under appropriate conditions, mismatch can prevent, or reduce polymerase extension (Prossner (1993) Tibtech. 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini, et al. (1992) Mol. Cell Probes 6:1).

In another embodiment, identification of the lineage-specific allelic variant is carried out using an oligonucleotide ligation assay (OLA), as described, e.g., in U.S. Pat. No. 4,998,617 and in Landegren, U., et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A., et al. have described a nucleic acid detection assay that combines attributes of PCR and OLA (Nickerson, D. A., et al., (1990) Proc. Natl. Acad. Sci. USA 87:8923-8927. In this method, PCR is used to achieve the exponential amplification of target DNA, which is then detected using OLA.

Several techniques based on this OLA method have been developed and can be used to detect specific lineage-specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe, et al. (1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Other techniques for the identification of allelic variants, e.g. SNPs, have been developed. These methods utilize matrix-assisted laser desorption/ionization time-of-flight mass spectrometry (MALDI-TOF MS). Several strategies for allele-discrimination (hybridization, cleavage, ligation, and primer extension) have been combined with MALDI-TOF mass spectrometric detection, and all of them allow high-throughput and/or automated genotyping of large numbers of SNPs (for a review, see, e.g., Gut, I. G. (2004) Hum Mutat. 23(5):437-41).

The invention further provides methods for detecting single nucleotide polymorphisms in a gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. However, any of the methods described above for detecting lineage-specific allelic variants of a polymorphic region of a gene can also be used to detect single nucleotide polymorphisms in a gene. Nevertheless, several methods have been developed to facilitate the analysis of such single nucleotide polymorphisms.

In one embodiment, the single base polymorphism can be detected using a specialized exonuclease-resistant nucleotide, as disclosed, e.g., in Mundy, C. R. (U.S. Pat. No. 4,656,127). According to the method, a primer complementary to the allelic sequence immediately 39 to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymnorphic site (Cohen, D., et al. (French Patent 2,650,840; PCT Application No. WO91/02087). As in the Mundy method of U.S. Pat. No. 4,656,127, a primer is employed that is complementary to allelic sequences immediately 3′ to a polymnorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P., et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymnorphic site of the target molecule being evaluated. In contrast to the method of Cohen, et al. (French Patent 2,650,840; PCT Appln. No. WO91/02087) the method of Goelet, P., et al. is preferably a heterogeneous phase assay, in which the primer or the target molecule is immobilized to a solid phase.

Several primer-guided nucleotide incorporation procedures for assaying polymorphic sites in DNA have been described (Komher, J. S., et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N., et al., Proc. Natl. Acad. Sci., USA 88:1143-1147 (1991); Prezant, T. R., et al., Hum. Mutat. 1:159-164 (1992); Ugozzoli, L., et al., GATA 9:107-112 (1992); Nyren, P., et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J Hum. Genet. 52:46-59 (1993)).

A number of template dependent processes are available to amplify the target sequences of interest present in a sample. One of the best known amplification methods is the polymerase chain reaction (PCR) which is described in detail in Mullis, et al., U.S. Pat. No. 4,683,195, Mullis, et al., U.S. Pat. No. 4,683,202, and Mullis, et al., U.S. Pat. No. 4,800,159, and in Innis et al., PCR Protocols, Academic Press, Inc., San Diego Calif., 1990. Briefly, in PCR, two primer sequences are prepared which are complementary to regions on opposite complementary strands of the target sequence. An excess of deoxynucleoside triphosphates are added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction products and the process is repeated. The target of the invention may be genomic DNA, RNA and/or cDNA. Preferably a reverse transcriptase PCR (RT-PCR) amplification procedure is performed to produce cDNA in order to quantify, e.g., measure, the amount of RNA amplified, e.g., assay the level of lineage-specific RNA.

PCR based methods can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Other techniques are known in the art to allow multiplex analyses of a plurality of markers.

PCR has been discussed above as a preferred method of initially amplifying target DNA, RNA and/or cDNA although the skilled person will appreciate that other methods may be used instead of or in combination with PCR. A recent development in amplification techniques which does not require temperature cycling or use of a thermostable polymerase is Self Sustained Sequence Replication (3SR). 3SR is modeled on retroviral replication and may be used for amplification (see for example Gingeras, T. R., et al. Proc. Natl. Acad. Sci., USA 87:1874-1878 and Gingeras, T. R., et al. PCR Methods and Applications Vol. 1, pp 25-33).

Alternative amplification methods include: self sustained sequence replication (Guatelli, J. C., et al., 1990, Proc. Natl. Acad. Sci. USA 87:1874-1878), transcriptional amplification system (Kwoh, D. Y., et al., 1989, Proc. Natl. Acad. Sci. USA 86:1173-1177), Q-Beta Replicase (Lizardi, P. M., et al., 1988, Biol. Technology 6:1197), and self-sustained sequence replication (Guatelli et al., (1989) Proc. Nat. Acad. Sci., USA 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

Amplification products may be assayed and/or detected in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, hybridization, sequencing, and the like. For determining the identity of the allelic variant of a polymorphic region located in the coding region of a gene corresponding to, for example, the genes corresponding to the molecules listed in Tables 4, 6, 7, and 8, yet other methods than those described above can be used. For example, identification of an allelic variant which encodes a mutated protein, e.g., a mutated protein corresponding to a molecule listed in Tables 4, 6, 7, and 8, can be performed by using an antibody specifically recognizing the mutant protein in, e.g., immunohistochemistry or immunoprecipitation. Antibodies to wild-type or mutated forms of the proteins can be prepared according to methods known in the art.

Alternatively, one can also measure an activity of a protein, such as binding to a ligand. Binding assays are known in the art and involve, e.g., obtaining cells from a subject, and performing binding experiments with a labeled lipid, to determine whether binding to the mutated form of the protein differs from binding to the wild-type of the protein.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific allelic variant.

Sample nucleic acid to be analyzed by any of the above-described methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi.

Another aspect of the invention pertains to the use of isolated nucleic acid molecules which are antisense to the nucleotide sequences of SEQ ID Nos: 1, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, e.g., the entire coding strand corresponding to a molecule listed in Tables 4, 6, 7, and 8, or to only a portion thereof In one embodiment, an antisense nucleic acid molecule is antisense to a “coding region” of the coding strand of a nucleotide sequence encoding a gene, e.g., a gene corresponding to a molecule listed in Tables 4, 6, 7, and 8. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding, e.g., a molecule listed in Tables 4, 6, 7, and 8. The term “noncoding region” refers to 5′ and 3′ sequences which flank the coding region that are not translated into amino acids (also referred to as 5′ and 3′ untranslated regions).

Given the coding strand sequences encoding, e.g. a molecule listed in Tables 4, 6, 7, and 8, disclosed herein, antisense nucleic acids of the invention can be designed according to the rules of Watson and Crick base pairing. The antisense nucleic acid molecule can be complementary to the entire coding region of a mRNA, e.g., a mRNA corresponding to the molecules listed in Tables 4, 6, 7, and 8, but more preferably is an oligonucleotide which is antisense to only a portion of the coding or noncoding region of, e.g., mRNA corresponding to a molecule listed in Tables 4, 6, 7, and 8. For example, the antisense oligonucleotide can be complementary to the region surrounding the translation start site of, e.g. mRNA corresponding to a molecule listed in Tables 4, 6, 7, and 8. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (ie., RNA transcribed from the inserted nucleic acid will be of an antisense orientation to a target nucleic acid of interest).

In yet another embodiment, identifying and/or detecting allelic variants, e.g., lineage-specific allelic variants, may be accomplished by any of a variety of sequencing reactions known in the art to directly sequence a gene of interest or a fragment thereof, generated by the methods of the invention. By comparing the sequence of the sample sequence, e.g., a biological sample isolated from a subject, e.g., a recipient, and comparing that sequence with the corresponding reference (control) sequence e.g., the nucleotide sequences set forth in SEQ ID Nos:1, 9, 11, 13, 15, 17, 19, 21, 23, 25, 27, 29, 31, 33, 35, 37, 39, 41, 43, 45,47, 49, 51, 53, 55, 57, 59, 61, 63, 65, 67, 69, 71, 73, 75, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 330, 332, 334, 336, 338, 340, 342, 344, 346, 348, 350, 352, 354, 356, 358, 360, 362, 364, 366, 368, 370, 372, 374, 378, and 380, or alternatively the nucleotide sequence of the donor, it may be determined whether an allelic variant is identified, or as a method to evaluate the effectiveness or clinical outcome of progenitor cell transfer. Exemplary sequencing reactions include those based on techniques developed by Maxam and Gilbert (Proc. Natl. Acad. Sci., USA (1977) 74:560) or Sanger (Sanger et al. (1977) Proc. Nat. Acad. Sci., USA 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No.5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

A preferred sequencing method is disclosed, e.g., in U.S. Pat. No. 6,258,568 entitled “Method of sequencing DNA based on the detection of the release of pyrophosphate and enzymatic nucleotide degradation”, referred to herein as “pyrosequencing”.

Clinical Course of Therapy

The invention thus relates to methods for identifying lineage-specific variants identified as described herein in combination with each other or in combination with other lineage-specific allelic variants in a gene of interest. The lineage-specific allelic variants may be further utilized to determine the most appropriate and effective clinical course of therapy for a subject and/or to determine or predict clinical outcome of a subject, e.g., following transplantation. Accordingly, the methods of the invention are particularly useful in monitoring the effectiveness of progenitor cell (e.g., transgenic cell or stem cell, e.g., bone marrow-derived stem cell and/or hematopoietic stem cell) transfer. In one embodiment, a method is featured to monitor the effectiveness of progenitor cell transfer in a subject, e.g., a mammal, such as a human, by identifying lineage-specific mRNA in the subject.

In another embodiment, the present invention provides a method for monitoring, e.g., evaluating, the effectiveness of progenitor cell transfer in a subject suffering from a disease or disorder comprising the steps of obtaining a biological sample from said subject and identifying lineage-specific mRNA in said biological sample. In another embodiment, functional engraftment of lineage-specific cells is monitored.

The methods of the invention may be utilized in a subject suffering from any disease or disorder which would benefit from progenitor cell transfer, including, but not limited to, stem cell therapy, e.g., embryonic stem cell therapy. In one embodiment, the disease or disorder is selected from the group consisting of hemoglobinopathies, e.g., thalassemias, sickle cell anemia, hemolytic anemia, hereditary elliptdcytosis, hereditary stomatocytosis, Chronic Granulomatous Disease, Chediak-Higashi syndrome, myelodysplasia, acute erythroleukemia, Kostmann's syndrome, infant malignant osteopetrosis, severe combined immunodeficiency, Wiskott-Aldrich syndrome, aplastic anemia, Blackfan Diamond anemia, Gaucher's disease, Hurler's syndrome, Hunter's syndrome, infantile metachromatic leukodystrophy, autoimmune disorders; and, any disease or disorder that would benefit from treatment by gene therapy, for example, Cystic Fibrosis, hemophilia, Gaucher's disease, numerous cancers associated with oncogenes, such as, breast, prostate, and colon cancer, diabetes mellitus, and organ failure or injury, e.g., cardiac, brain, lung, liver, renal, prostate or pancreas organ failure or injury.

In another embodiment, the methods of the invention may be utilized in a subject suffering from cognitive and neurodegenerative disorders, such as, for example, Alzheimer's disease, dementias related to Alzheimer's disease (such as Pick's disease), Parkinson's and other Lewy diffuse body diseases, senile dementia, Huntington's disease, Gilles de la Tourette's syndrome, musculoskeletal diseases, multiple sclerosis, amyotrophic lateral sclerosis, progressive supranuclear palsy, epilepsy, and Jakob-Creutzfieldt disease. Additional diseases and disorders which may benefit from progenitor cell transfer, e.g., stem cell therapy, are described in, for example, Roybon L, et al. (2004) Cell Tissue Res. August 11 [epub ahead of print]; McKay R D (2004) B Biol Sci. 359(1445):851-6; Lindvall O, et al. (2004) Nat Med. 10 Suppl:S42-50; Atala A.(2004) Rejuvenation Res. 7(1):15-31; Hussain M A, Theise N D. (2004) Lancet 364(9429):203-5; Silani V, et al. (2004) Lancet. 364(9429):200-2; Mathur A, Martin J F. (2004) Lancet 364(9429):183-92; and Rice C M, Scolding N J. (2004) Lancet 364(9429):193-9; Taguchi, et al. (2004) J. of Clin. Invest. 114(3):330; and Neuringer et al. (2004) Respiratory Research 20 Jul. 2004; 5:6; Peterson (2004) J. of Clin. Invest. 114(3):312, the entire contents of which are incorporated herein by reference.

The methods described herein can be used alone, or in combination with other clinical/diagnostic methods, including but not limited to implementation of lifestyle changes (e.g., changes in diet or environment), administration of medication, e.g., immunosuppressive agents, cellular therapy, such as lymphocyte infusion, use of medical devices, or, surgical procedures, cell transplantation, e.g., allogenic or autologous, e.g. myeloablative or nonmyeloablative, administration of a therapeutic agent to an isolated cell or tissue or cell line from a subject, or any combination thereof. Information obtained using the methods described herein alone or in combination with information from other diagnostic analyses is useful for determining subsequent clinical courses of action. For example, a method to quantify lineage-specific chimerism following progenitor cell transplantation for treatment of disease or disorders, for example but not limited to, hemoglobinopathies would provide a means of determining what percent of donor derived lineage-specific cells, e.g., transgenic cells or stem cells, e.g., bone marrow-derived stem cells and/or hematopoietic stem cells, are needed in order to correct the symptoms related to the disease or disorder, e.g., hemoglobinopathy.

If, for example, a subject has received a progenitor cell transfer for the treatment of a sickle cell syndrome, and it is determined that the level of lineage-specific RNA is inadequate to alleviate disease symptoms and promote erythropoiesis, a second transfer of stem cells or an infusion of donor lymphocytes may be indicated. It may also be determined that the level of lymphoid and/or myeloid cells is insufficient to prevent, for example, infection, and thus a second transfer of stem cells or an infusion of donor lymphocytes may be indicated. For example, if the level of donor erythropoiesis was decreasing on serial chimerism measurements, e.g., serial determinations of quantifying lineage-specific cells, then additional treatment would be desired in an to attempt to alleviate disease symptoms and promote erythropoiesis. Alternatively, no further progenitor cell transfer may be indicated if the level of lineage-specific RNA is adequate to alleviate disease symptoms and promote, for example, immune cell function and/or erythropoiesis. It will be recognized that the methods described in the present application are useful to continuously monitor the level of lineage-specific chimerism at multiple-time intervals following transplantation, including but not limited to, 1, 10, 20, 30, 40, 60, 80, 100, 150, 200, 250, 300 days, or 1, 2, 3, 5, 7, 10, 15, 20, 25, 30, 35, 40, 45, 50, 75, 100 years.

Arrays

The methods of the invention may be carried out using chip-based or array-based methods, wherein a panel of allelic variants, e.g., two or more allelic variants, or SNPs, are identified, detected, and/or quantified. As used herein, the terms “array” or “chip”, used interchangeably herein, represent an intentionally created collection of molecules which can be prepared either synthetically or biosynthetically. In particular, the term “array” as used herein means an intentionally created collection of peptides, proteins, oligonucleotides or polynucleotides attached to at least a first surface of at least one substrate wherein the identity of each molecule at a given predefined region is known.

To create the arrays of the invention, single-stranded nucleic acid molecules, e.g., polynucleotide probes, can be spotted onto a substrate in a two-dimensional matrix or array. Each single-stranded polynucleotide probe can comprise at least 5, 10, 15, 20, 25, 30, 35, 40, or 50 or more contiguous nucleotides. In one embodiment, the polynucleotide probes can be selected from the nucleotide sequences shown in Tables 1 or 9.

The invention also includes an array comprising a molecule of the present invention e.g., some or all of the sets of molecules set forth in Tables 4, 6, 7, and 8, complements or fragments thereof. The array can be used to assay expression of one or more genes in the array. In one embodiment, the array can be used to detect and/or identify lineage-specific allelic variants, which is useful to determine a clinical course of therapy, determining immune cell reconstitution, and/or clinical outcome in a subject, following progenitor cell transfer, as described above.

A. Preparation of Arrays

Arrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the array is a matrix in which each position represents a discrete binding site for a product encoded by a gene (e.g., a protein or RNA), and in which binding sites are present for products of most or almost all of the genes in the organism's genome. In one embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

B. Preparing Nucleic Acid Molecules for Arrays

As noted above, the “binding site” to which a particular cognate cDNA specifically hybridizes is usually a nucleic acid or nucleic acid analogue attached at that binding site. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e., fragments that do not share more than 10 bases of contiguous identical sequence with any other fragment on the array). Computer programs are useful in the design of primers with the required specificity and optimal amplification properties. See, e.g., Oligo version 5.0 (National Biosciences™). In the case of binding sites corresponding to very long genes, it will sometimes be desirable to amplify segments near the 3′ end of the gene so that when oligo-dT primed cDNA probes are hybridized to the array, less-than-full length probes will bind efficiently. Typically each gene fragment on the array will be between about 50 bp and about 2000 bp, more typically between about 100 bp and about 1000 bp, and usually between about 300 bp and about 800 bp in length. PCR methods are well known and are described, for example, in Innis et al. eds., 1990, PCR Protocols: A Guide to Methods and Applications, Academic Press Inc. San Diego, Calif., which is incorporated by reference in its entirety. It will be apparent-that computer controlled robotic systems are useful for isolating and amplifying nucleic acids.

An alternative means for generating the nucleic acid molecules for the array is by synthesis of synthetic polynucleotides or oligonucleotides, e.g., using N-phosphonate or phosphoramidite chemistries (Froehier et al. (1986) Nucleic Acid Res 14:5399-5407; McBride et al. (1983) Tetrahedron Lett. 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid molecule analogues may be used as binding sites for hybridization. An example of a suitable nucleic acid analogue is peptide nucleic acid (see, e.g., Egholm et al. (1993) Nature 365:566-568; see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al. (1995) Genomics 29:207-209). In yet another embodiment, the polynucleotide of the binding sites is RNA.

C. Attaching Nucleic Acid Molecules to the Solid Surface

The nucleic acid molecule or analogue are attached to a solid support, which may be made from glass, plastic (e.g., polypropylene, nylon), polyacrylamide, nitrocellulose, or other materials. An example of a method for attaching the nucleic acid molecules to a surface is by printing on glass plates, as is described generally by Schena et al. (1995) Science 270:467-470, the contents of which are expressly incorporated herein by reference. This method is especially useful-for preparing arrays of cDNA. See also DeRisi et al. (1996) Nature Genetics 14:457-460; Shalon et al. (1996) Genome Res. 6:639-645; and Schena et al. (1995) Proc. Natl. Acad. Sci. USA 93:10539-11286. Each of the aforementioned articles is incorporated by reference in its entirety.

A second example of a method for making arrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., (1991) Science 251:767-773; Pease et al., (1994) Proc. Natl. Acad. Sci. USA 91:5022-5026; Lockhart et al. (1996) Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al. (1996) Biosensors & Bioelectronics 11: 687-90). When these methods are used, oligonucleotides (e.g., 20-mers) of known sequence are synthesized directly on a surface such as a derivatized glass slide. In one embodiment, the array produced is redundant, with several oligonucleotide molecules per RNA. Oligonucleotide probes can be chosen to detect alternatively spliced mRNAs.

Other methods for making arrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is hereby incorporated in its entirety), could be used, although, as will be recognized by those of skill in the art, very small arrays will be preferred because hybridization volumes will be smaller.

Another method for making arrays is to directly deposit the probe on to the array surface. In such an embodiment probes will bind non-covalently or covalently to the array depending on the surface of the array and characteristics of the probe. In preferred embodiments the array has an epoxy coating on top of a glass microscope slide and the probe is modified at the amino terminal by an amine group. This combination of array surface and probe modification results in the covalent binding of the probe. Other methods of coating the array surface include using acrylamide, sialinization and nitrocellulose. There are several methods for direct deposit of the probes on to the array surface. In one embodiment, the probes are deposited using a pin dispense technique. In this technique, pins deposit probes onto the surface either using contact or non-contact printing. One preferred embodiment is non-contact printing using quill tip pins. Another embodiment uses piezo electric dispensing to deposit the probes.

Control composition may be present on the array including compositions comprising oligonucleotides or polynucleotides corresponding to genomic DNA, housekeeping genes, negative and positive control genes, and the like. These latter types of compositions are not “unique”, i.e., they are “common.” In other words, they are calibrating or control genes whose function is not to tell whether a particular “key” gene of interest is expressed, but rather to provide other useful information, such as background or basal level of expression. The percentage of samples which are made of unique oligonucleotides or polynucleotide that correspond to the same type of gene is generally at least about 30%, and usually at least about 60% and more usually at least about 80%.

Kits

The present invention also features kits to identify lineage-specific mRNA. In one embodiment, the kit comprises primers for the amplification of lineage-specific mRNA and instructions for use of those primers to identify lineage-specific mRNA. In one embodiment, the primers correspond to those in SEQ ID Nos. 3 and 5. In another embodiment, the primers correspond to those in SEQ ID Nos. 6 and 8. The kit may comprise a box or container that holds the components of the kit. The box or container is affixed with a label or a Food and Drug Administration approved protocol. The box or container holds components of the invention that are preferably contained within plastic, polyethylene, polypropylene, ethylene, or propylene vessels. The vessels can be capped-tubes or bottles.

In one embodiment, kits are provided that comprise at least one probe or primer which is capable of specifically hybridizing under stringent conditions to one or more polymorphic regions, e.g., one or more polymorphic regions corresponding to a molecule listed in Tables 4, 6, 7, and 8, and instructions for use. The kits may comprise at least one of nucleic acids of SEQ ID Nos: 3-8. Preferred kits for amplifying at least a portion of a gene, e.g., a gene corresponding to a molecule listed in Tables 4, 6, 7, and 8, comprise at least two primers, at least one of which is capable of hybridizing to a lineage-specific allelic variant sequence.

The kits of the invention can also comprise one or more control nucleic acids or reference nucleic acids, such as nucleic acids comprising an intronic sequence, e.g., an intronic sequence corresponding to a molecule listed in Tables 4, 6, 7, and 8. For example, a kit can comprise primers for amplifying a polymorphic region of a gene and a control DNA corresponding to such an amplified DNA and having the nucleotide sequence of a specific lineage-specific allelic variant. Thus, direct comparison can be performed between the DNA amplified from a subject and the DNA having the nucleotide sequence of a lineage-specific allelic variant. In one embodiment, the control nucleic acid comprises at least a portion of a gene corresponding to a molecule listed in Tables 4, 6, 7, and 8 of an individual who does not have a disease associated with an allelic variant of a gene listed in Tables 4, 6, 7, and 8, or a disease or disorder associated with an aberrant activity of a protein encoded by a gene corresponding to the molecules listed in Tables 4, 6, 7, and 8.

Yet other kits of the invention comprise at least one reagent necessary to perform the assay. For example, the kit can comprise an enzyme. Alternatively the kit can comprise a buffer or any other necessary reagents.

In another embodiment, the invention provides a kit for amplifying and/or for determining the molecular structure of at least a portion of a gene, e.g., a gene corresponding to a molecule listed in Tables 4, 6, 7, and 8, comprising a probe or primer capable of hybridizing to a gene and instructions for use. In one embodiment, determining the molecular structure of a region of a gene comprises determining the identity of the allelic variant of the polymorphic region. Determining the molecular structure of at least a portion of a gene can comprise determining the identity of at least one nucleotide or determining the nucleotide composition, e.g., the nucleotide sequence of a gene corresponding to a molecule listed in Tables 4, 6, 7, and 8.

The kit may, optionally, also include DNA sampling means. DNA sampling means are well known to one of skill in the art and can include, but not be limited to substrates, such as filter papers, the AmpliCard™. (University of Sheffield, Sheffield, England S10 2JF; Tarlow, J W, et al., J. Invest. Dernatol. 103:387-389 (1994)) and the like; RNA purification reagents, lysis buffers, proteinase solutions and the like; RT- and PCR reagents, such as 10× reaction buffers, reverse transcriptase, thermostable polymerase, dNTPs, and the like; and allele detection means such as the HinfI restriction enzyme, allele specific oligonucleotides, degenerate oligonucleotide primers for nested PCR. PCR amplification oligonucleotides should hybridize between 25 and 2500 base pairs apart, preferably between about 100 and about 500 bases apart, in order to produce a PCR product of convenient size for subsequent analysis. The assay kit and method may also employ labeled oligonucleotides to allow ease of identification in the assays, e.g., sequencing, e.g., pyrosequencing. Examples of labels which may be employed include radio-labels, enzymes, fluorescent compounds, streptavidin, avidin, biotin, magnetic moieties, metal binding moieties, antigen or antibody moieties, and the like.

### EXAMPLES

This invention is further illustrated by the following examples which should not be construed as limiting. The contents of all references, sequence listing, figures, Accession Numbers, patents and published patent applications cited throughout this application are hereby incorporated by reference.

### Example 1

### Methods to Quantify Engraftment of Donor Cells in Hematopoietic Cell Lineages Following Stem Cell Transplantation

A. Materials and Methods

Subject Samples and Cell Preparation

Heparinized blood samples from subjects were obtained prior to and at various times post-transplant, upon enrollment into IRB approved research protocols. Peripheral blood mononuclear cells (PBMC) from normal donors and subjects were isolated by Ficoll/Hypaque density gradient centrifugation, cryopreserved with 10% DMSO and stored in vapor phase liquid nitrogen until the time of analysis.

RNA Extraction and Reverse Transcription

RNA was extracted from 20×10⁶ PBMC by the single-step acid guanidinium thiocyanate/phenol/chloroform method (Trizol) according to the manufacturer's protocol (Invitrogen, Carlsbad, Calif.). First-strand cDNA was generated from 2 μg of total RNA using random hexanucleofides (Pharmacia LKB Biotechnology Inc., Picscataway, N.J.) and reverse transcriptase (Superscript; GIBCO BRL, Gaithersburg, Md.).

Genomic DNA Extraction

Genomic DNA was extracted from 1-3×10⁶ PBMC or bone marrow mononuclear cells according to the manufacturer's protocol (Wizard Genomic DNA Purification Kit, Promega, Madison, Wis.). Prior to amplification, all DNA samples were quantified by ultraviolet (UV) spectrophotometry and diluted to working concentrations.

PCR of the Sickle Mutation and Hemoglobin Polymorphisms

The nucleotide sequence of the isolated human β-globin cDNA and the predicted amino acid sequence of the human β-globin polypeptide are shown in SEQ ID NOs: 1 and 2, respectively. The nucleotide sequence of β-globin is also described in GenBank Accession No. GI: 28302128 (SEQ ID NO:1) (the contents of which are included herein by reference). A β-globin locus polymorphism (H3H) was identified from public databases at the NCI Genetic Annotation Initiative (see, for example, Clifford R., et al. (2000) Genome Res. 10:1259-65), available through GenBank. PCR primers were designed to flank the H3H polymorphism, which is a T to C silent substitution at nucleotide position 59, codon 3, of SEQ ID NO:1 or the sickle cell mutation, which is an A to T substitution at nucleotide position 70, codon 7, resulting in a Glutamic acid (E) to Valine (V) amino acid change. PCR primers were optimized for Mg concentration and annealing temperature (Table 1). For each set of primers, one of the primers was biotinylated for the pyrosequencing reaction (see below). Each 50 μl reaction mixture contained 3 μl of cDNA and the following concentrations of other components: 1× Taq Gold buffer (Applied Biosystems, Foster City, Calif.), MgCl₂ (in the concentration specified in Table 1), 400 nmol each primer, 200 nmol DATP, dCTP, dGTP, dTTP, and 2 units AmpliTaq Gold DNA polymerase (Applied Biosystems, Foster City, Calif.). One cycle of denaturation (95° C. for 10 min) was followed by 46 cycles of PCR (94° C. for 30 s, 55° C. for 30 s, 72° C. for 30 s), and finally extension at 72° C. for 10 minutes.

TABLE 1
Primers and probes used to amplify β-globin polymorphisms

| | | | | |
|---|---|---|---|---|
| Polymorphism/ | Amplicon | | Annealing | Mg |
| mutation | size | primer sequence | temp | conc. |

| | | | | | | |
|---|---|---|---|---|---|---|
| Template | Nucleotide | AA | (bp) | (5′-3′) | (° C.) | (mM) |

| | | | | | | |
|---|---|---|---|---|---|---|
| β-globin | A->T | E7V | 101 | For: B-TAG CAA CCT CAA ACA GAC | 55 | 3 |
| RNA | | | | ACC | | |
| (sickle) | | | | (SEQ ID NO:3) | | |
| | | | | Rev: TCA CCA CCA ACT TCA TCC | | |
| | | | | AC | | |
| | | | | (SEQ ID NO:4) | | |
| | | | | Probe: ACG GCA GAC TTC TC | | |
| | | | | (SEQ ID NO:5) | | |
| β-globin | A->T | E7V | 149 | For: B-GCA GGG AGG GCA GGA | 55 | 3 |
| gDNA | | | | (SEQ ID NO:6) | | |
| (sickle) | | | | Rev: GCA GTA ACG GCA GAC TTC | | |
| | | | | TC | | |
| | | | | (SEQ ID NO:7) | | |
| | | | | Probe: ACG GCA GAC TTC TC | | |
| | | | | (SEQ ID NO:8) | | |
| β-globin | T->C | H3H | 101 | For: B-TAG CAA CCT CAA ACA GAC | 55 | 3 |
| RNA | | | | ACC | | |
| | | | | (SEQ ID NO:77) | | |
| | | | | Rev: TCA CCA CCA ACT TCA TCC | | |
| | | | | AC | | |
| | | | | (SEQ ID NO:78) | | |
| | | | | Probe: TCT CCT CAG GAG TCA | | |
| | | | | (SEQ ID NO:79) | | |

B = biotinylated

Pyrosequencing

Biotinylated single-strand DNA fragments were generated by mixing the PCR product with streptavidin-coated paramagnetic beads (Dynalbeads M280; Dynal, Norway) and processed according to the manufacturer's instructions (Pyrosequencing Sample Preparation; Pyrosequencing AB, Uppsala, Sweden). Throughout the sample preparation steps, the immobilized fragments coupled to the beads were moved using a magnetic tool (PSQ 96 Sample Preparation Tool; Pyrosequencing AB). An automated pyrosequencing instrument, PSQ96 (Pyrosequencing AB), was used to determine cDNA sequence. The reaction was carried out at room temperature. The sequencing protocol utilized stepwise elongation of the primer strand by sequential addition of the deoxynucleoside triphosphates and the simultaneous degradation of the residual unincorporated nucleotides. As the sequencing reaction continued, extension of the CDNA strand by successful nucleotide incorporation results in the release of light and the DNA sequence is determined from the peaks in the pyrogram using Pyrosequencing software (Pyrosequencing AB).

To examine the linearity of pyrosequencing output, plasmids bearing the normal or sickle β-globin gene were mixed at different concentrations between 0% and 100% normal donor. The mixture of plasmids was then PCR amplified as described above and % sickle mutated β-globin was determined by Pyrosequencing. The percentage of hematopoietic chimerism was determined by the PSQ96 Allele Discrimination Software (Pyrosequencing AB). These plasmids were generated by cloning the β-globin insert amplified from normal donor and sickle cell subject cDNA and confirmed to be identical to published β-globin sequence, into pCR2.1-TOPO (Invitrogen, Carlsbad, Calif.). Similarly, known mixtures of cDNA from PBMC of 2 normal individuals with disparate genotypes at the β-globin loci H3H were also used to demonstrate linearity of the pyrosequencing output.

X and Y Chromosome FISH (BioView Analysis) for Quantitation of Erythroid Precursor Engraftment

The BioView technique has been described in detail in Shimoni A., et al. (2002) Leukemia 16:1413-1418 and 14-19-1422. Briefly, bone marrow collected in EDTA was diluted 1:1 in PBS and layered on Ficoll/Hypaque to collect mononuclear cells. A cytospun slide was prepared from 300 μl of the final cell suspension containing 30,000 cells. The slides were air-dried, stained with May-Grunwald-Giemsa (MGG), and analyzed by automated bright field scanning microscope (Axioplan2; Carl Zeiss, Jena, Germany) in conjunction with a 3CCD progressive scan camera (DXC9000, Sony, Tokyo, Japan). This system saves the coordinates and images of all cells found on the slides for future reference during the next phases of analysis and classifies the cells according to their morphology after MGG staining. All cell classification assignments were confirmed by a hematopathologist for accuracy. Subsequently, MGG stain was removed using ice-cold methanol/acetic acid (3:1) for 1 hour and washed with PBS. Enzymatic treatment was performed using digestion enzyme solution (Bio-Blue, Bioview) for 7 minutes at 37° C., followed by three 5 minute washes in PBS. Slides were dehydrated in ethanol, dried at 37° C. for 5 minutes, and hybridized with X (X centromeric sequence probe, Spectrum Green, Vysis, Downer's Grove, Ill.) and Y (chromosome Y satellite imi sequence probe; Spectrum Orange, Vysis, Downer's Grove, Ill.) probes. Slides and probe were denatured at 71° C. for 3 minutes and hybridized overnight at 42 C using a HYBrite system (Vysis, Downer's Grove, Ill.). Following hybridization, slides were washed (0.4×SSC at 73° C. for 2 minutes, followed by 2 minutes in 0.01% NP-40/2×SSC) at room temperature and then stained with BioView counterstain (DAPI). Finally, automated fluorescence scanning of the slides was performed and positions and signals saved. Using this approach, the morphologic features of each cell could be directly compared to its XY FISH pattern and donor vs. recipient assignment for erythroid and other lineages could be made.

ABO Immunohistochemistry for Quantitation of Erythroid Precursor Engraftment

Immunohistochemistry with anti-B-antigen monoclonal antibody (DAKO USA, Carpinteria, Calif.) was performed using bone marrow mononuclear cells that were cytospun onto glass slides as described above. Slides were fixed in cold (−20 C) acetone for 4 minutes and then air-dried. Next, slides were pre-treated with Peroxidase Block (DAKO USA, Carpinteria, Calif.) for 5 minutes to quench endogenous peroxidase activity, followed by a 1:5 dilution of goat serum in 50 Mm Tris-Cl, pH 7.4, for 20 minutes to block non-specific binding sites. Monoclonal anti-human B-antigen antibody was applied at a 1:50 dilution in 50-mM Tris-Cl, pH 7.4 with 3% goat serum for 1 hour. Slides were washed in 50-mM Tris-Cl, 0.05% Tween 20, pH 7.4, and goat anti-mouse horseradish peroxidase-conjugated antibody (BioView, Nes Ziona, Israel) was applied for 30 minutes. After further washing, immunoperoxidase staining was developed using a chromogen kit (BioView) per the manufacturer.

B. Results

Specific Amplification of β-Globin mRNA to Assess Erythroid Lineage Engraftment

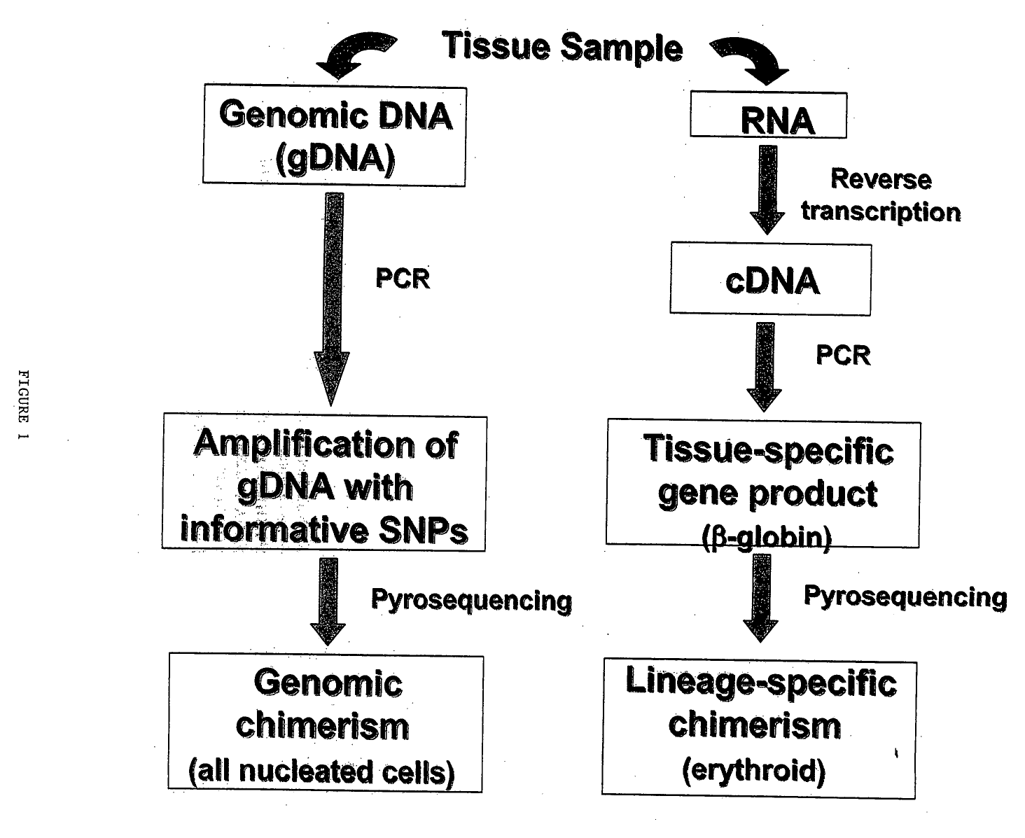

This example describes the development of a method to monitor erythroid lineage-specific engraftment that can be applied to, for example, monitoring the effects of allogenic transplant on nonmalignant hematological diseases and disorders, e.g., sickle cell disease. In nonmalignant hematological diseases and disorders, donor red blood cells will have a normal life span while subject-derived red blood cell production will continue to be ineffective. In addition, subject-derived red blood cells will continue to undergo hemolysis. As a result, the level of engraftment of donor red blood cell precursors is likely to be quite different than the level of functional red blood cell engraftment in peripheral blood. The traditional methods of red blood cell antigen typing or assessment of hemoglobin by electrophoresis can distinguish recipient from donor red blood cells but these methods are limited by the frequent transfusions administered to almost all subjects in the peri-transplant period and the long life span of transfused red blood cells. A schematic outline of the methods of the invention comparing the present RNA-based method to a DNA-based method for measurement of donor engraftment is presented in FIG. 1. Whereas genomic DNA-based methods measure chimerism in all nucleated cells, the RNA-based method of the invention is restricted to the assessment of expressed genes that are unique to specific cell lineages and therefore provides a method for selectively examining lineage-specific chimerism.

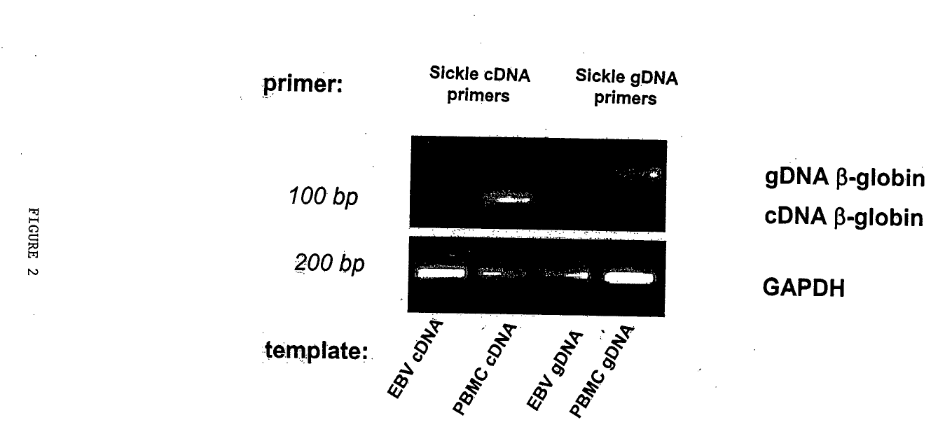

The sickle mutation caused by a single base pair substitution of glutamic acid to valine in the β-globin gene represents an informative polymorphism that distinguishes homozygous recipient DNA from heterozygous donor DNA. Pyrosequencing of PCR-amplified genomic DNA can distinguish homozygous recipient DNA from either heterozygous or alternate allele homozygous donor DNA and provide a quantitative measurement of each allele in the sample. While the β-globin gene is present in all nucleated cells, β-globin RNA is only expressed in erythroid-lineage cells. As shown in FIG. 1, DNA pyrosequencing of a sequence containing the sickle mutation provides a molecular assessment of chimerism of all nucleated cells. In contrast, RNA pyrosequencing of the same mutation provides a specific assessment of erythroid lineage chimerism. As shown in FIG. 2, PCR amplification of genomic DNA with gene-specific primers resulted in the amplification of β-globin DNA from cells derived from different lineages including EBV-transformed B cell lines and PBMC. In contrast, the cDNA primers specifically amplified β-globin only from normal donor PBMC, which contains erythroid precursors. β-globin cDNA was not detected following reverse transcription of RNA from EBV-transformed B cells. The amplified product from PBMC cDNA was confirmed to be β-globin sequence by direct DNA sequencing. Therefore, RNA pyrosequencing is a quantitative sequencing method to detect the presence of single nucleotide polymorphisms (SNPs) within coding regions of β-globin mRNA, and provides a rapid and accurate assessment of reconstitution of erythroid lineage cells after allogeneic transplantation.

Pyrosequencing of β-Globin Gene Polymorphisms/Mutations

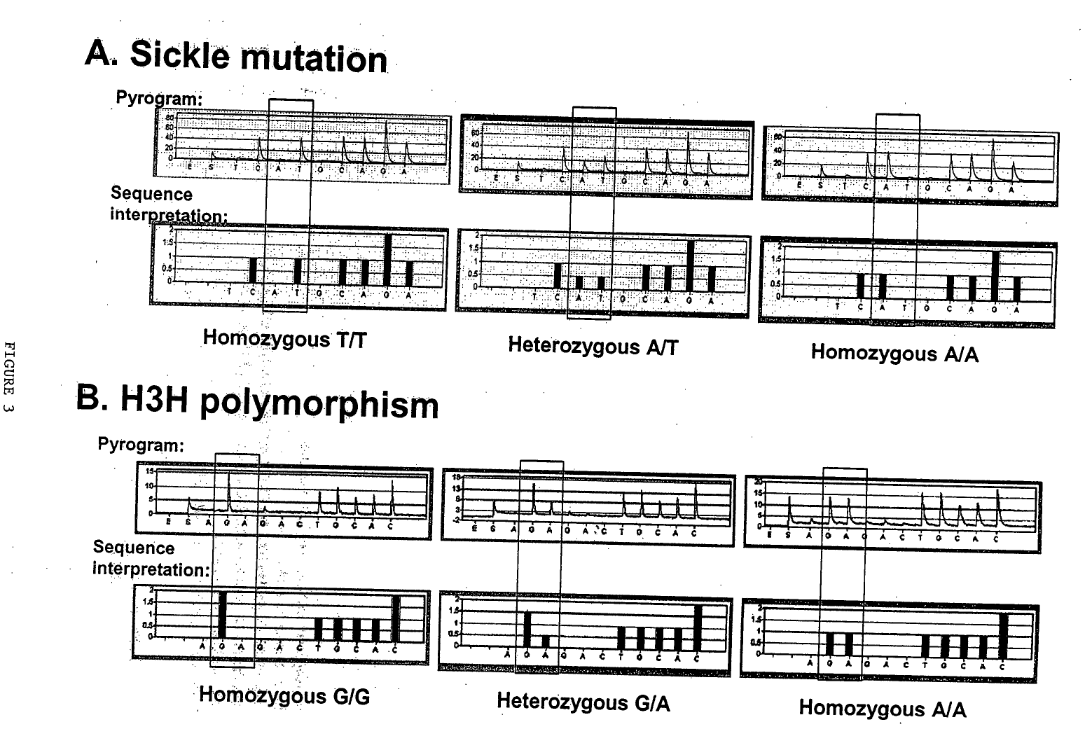

To determine whether pyrosequencing could reliably distinguish between β-globin RNA derived from normal donors, or individuals heterozygous (sickle cell trait) or homozygous (sickle cell disease) for the sickle mutation, RNA-genotyping was determined for 5 normal individuals, 2 individuals with sickle trait, and 3 subjects with sickle cell disease. As shown for representative samples in FIG. 3A, sickle mutation genotyping of β-globin RNA reliably identifies homozygous, heterozygous and normal individuals. Normal individuals are homozygous T/T at nucleotide position 17 of the β-globin gene and are represented by a double-height peak for T (top left panel). Subjects with sickle cell disease have a double height peak for A at the same position (top right panel) and heterozygotes with sickle trait have single height peaks for both A and T (top center panel).

Using public databases, an additional polymorphism was identified in the β-globin gene (Table 1), at codon 3 (H3H). PCR primers and sequencing probes were designed, optimized, and tested on samples generated from reverse transcription of RNA from 16 normal volunteers of diverse racial backgrounds. As shown in FIG. 3B, pyrosequencing clearly distinguishes individuals who are heterozygous or homozygous at the H3H locus. Since the probe reads the complementary strand sequence G G/A TGCACC, individuals who are homozygous C/C are represented as a 2× height G peak (a G precedes the polymorphic locus) (left panel), heterozygotes are represented by a 1.5× height G peak and 0.5× height A peak (middle panel) and homozygous T/T individuals are represented by peaks of equal height between G and A.

Quantitative Assessment of Mixed Erythroid Lineage Chimerism

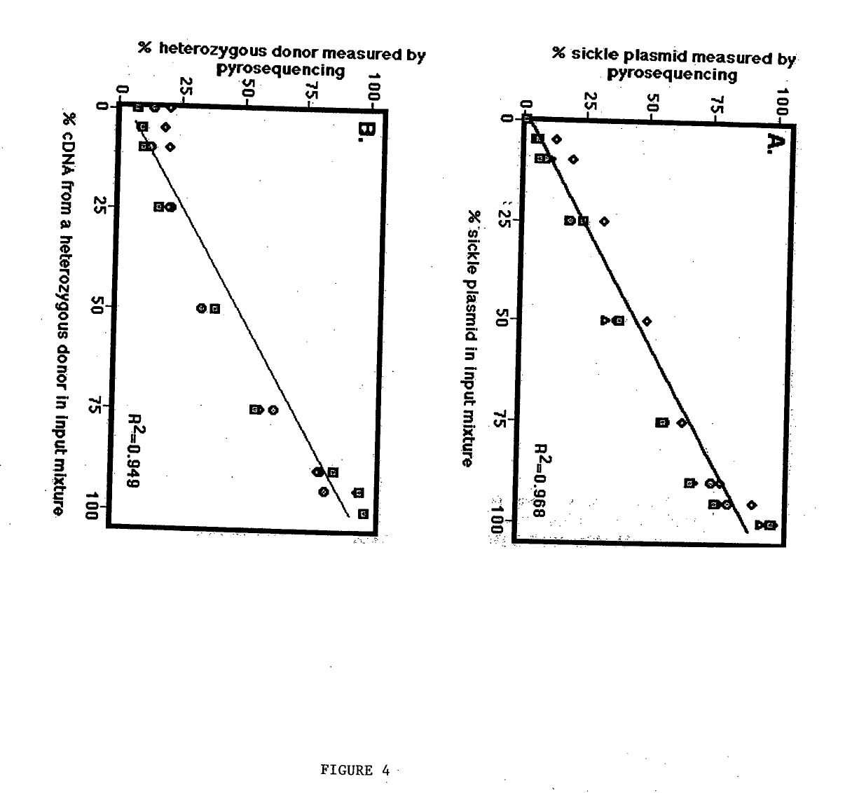

After establishing that pyrosequencing distinguishes recipient from donor-derived RNA, this method was tested to determine whether it could provide a quantitative assessment of erythroid lineage chimerism following allogenic HSCT. Plasmids encoding either normal or sickle β-globin were mixed at varying concentrations and pyrosequencing for the sickle mutation was used to measure the degree of chimerism. Additionally, cDNA derived from PBMC RNA of normal donors, one homozygous (C/C) and another heterozygous (C/T) at the β-globin polymorphism H3H, were mixed at varying concentrations, and similarly analyzed by pyrosequencing. Standard curves generated from analysis of plasmid DNA and normal donor cDNA mixtures are shown in FIGS. 4A and 4B, respectively. Pyrosequencing output was highly linear across all input frequencies for both the sickle mutation and the, β-globin H3H polymorphism (r²<0.968, and r²<0.9487 respectively). FIG. 4B shows that linearity is preserved even when the mixtures were generated from a combination of homozygous and heterozygous disparate donors. This is particularly relevant when monitoring post-transplant chimerism for sickle cell disease, since related donors will often be heterozygous for the sickle mutation.

Similar results were obtained when samples were processed at different times. Therefore, these experiments demonstrate that the RNA pyrosequencing method is both reliable and quantitative. Consistent with previous experience using gDNA as a template, it was found that pyrosequencing of cDNA accurately measured the presence of as little as 5% donor cells. However, at lower levels of chimerism, the measured level of donor cells was not consistently above background and donor cells were not detected in all replicates.

Assessment of Erythroid Lineage Chimerism Following Non-Myeloablative Allogenic Stem Cell Transplantation in Subject Samples

To demonstrate the utility of pyrosequencing RNA for the sickle mutation to quantify chimerism of erythroid lineage cells after allogenic HSCT, serial blood and marrow samples 5 subjects (3 with sickle cell disease) who underwent allogenic HSCT after non-myeloablative conditioning was examined. The clinical characteristics of the two subjects are described in Table 2. Results of erythroid lineage chimerism determined by pyrosequencing were compared to results of hematopoietic chimerism determined by 4 other methods: 1) conventional analysis of genomic DNA short tandem repeats (STR); 2) pyrosequencing of genomic DNA for informative β-globin polymorphisms; 3) FISH analysis of bone marrow cells with X and Y chromosome probes; and 4) hemoglobin electrophoresis.

TABLE 2
Clinical Characteristics of Subjects 1 and 2.

| | Subject 1 | Subject 2 |
|---|---|---|
| Subject Age/Sex | 34/F | 52/M |
| Donor sickle genotype/Sex | Sickle trait/M | Sickle trait/M |
| Subject/Donor ABO type | A/O | O/B |
| CD34+ cells/kg infused | 3.5 × 10⁶ | 7.25 × 10⁶ |
| Pre-transplant RBC exchange | 9 | 12 |
| transfusion (# units) | | |
| Post-transplant RBC units | 7 (d 21) | 2 (d 31) |
| transfused (day of last | | |
| transfusion) | | |

Subject 1 and 2 Histories

Subject 1 is a 34-year-old Nigerian woman with severe sickle cell disease and Subject 2 is 52-year-old man with both severe sickle cell disease and multiple myeloma. Both subjects received G-CSF mobilized peripheral stem cells from HLA-identical sibling donors. The donors for both subjects were male and had sickle trait. Subject 1 was ABO antigen type A, whereas the donor was type O. Subject 2 was ABO antigen type O, whereas his donor was type B. Both subjects underwent red blood cell exchange transfusion within two weeks prior to transplant, resulting in a decrease in hemoglobin S concentration from >75% to less than 35%. Subjects received fludarabine (30 mg/m²/day) and busulfan (Busulfex 0.8 mg/kg/day) for four days, and oral prednisone and FK506 for GVHD prophylaxis. Subject 1 received 3.5×10⁶ CD34+ cells/kg and Subject 2 received 7.25×10⁶ CD34+ cells/kg. Neither subject experienced treatment-related toxicities or GVHD following non-myeloablative conditioning and both subjects reported significant improvement in their sickle cell disease-related symptoms following transplantation. Subject 1 received 7 units of packed red blood cells (PRBC) and Subject 2 received 2 units of pRBC in the first month post transplant and did not receive any further transfusions.

Measurement of Hematopoietic Engraftment by Assessment of Genomic DNA

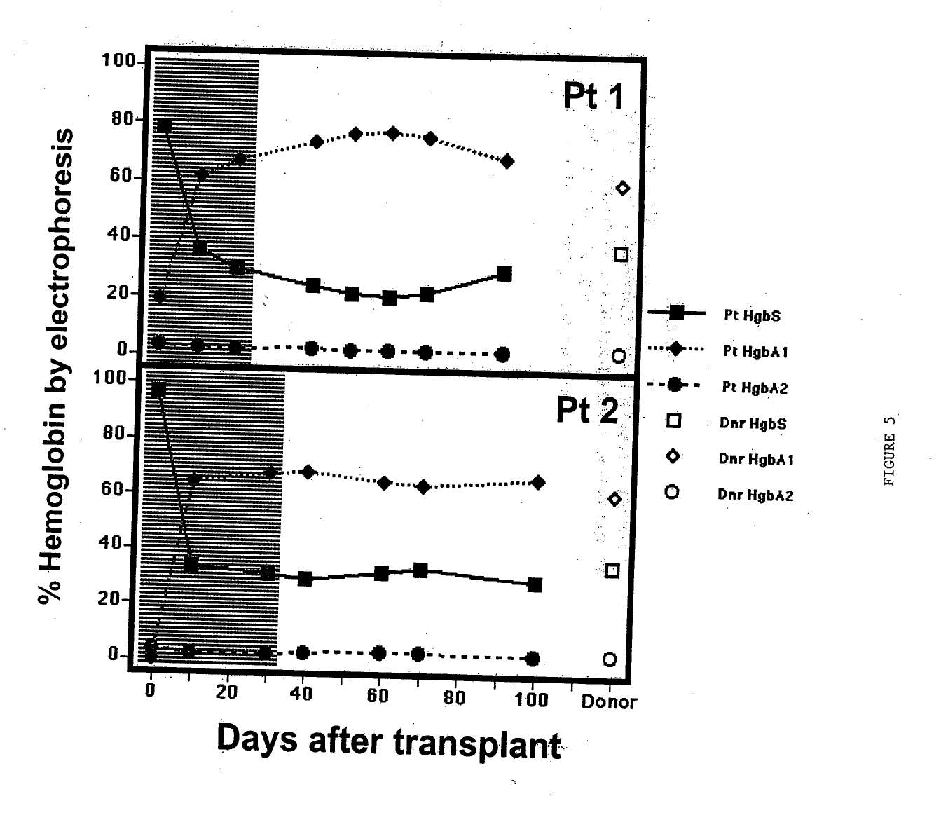

Hematopoietic engraftment was monitored by analysis of gDNA derived from PBMC and BM using pyrosequencing for β-globin polymorphisms. All subjects developed mixed gDNA chimerism, ranging from 18-50%, between days 60-180, with no differences between PBMC and BM levels. As shown in FIG. 5B, donor engraftment of Subject 1, measured by gDNA pyrosequencing, increased from 16% on day 10 to 25-30% from day 20 and onward. Subject 2 demonstrated a higher level of donor engraftment post transplant. By day 33, Subject 2 achieved 45% donor engraftment in PBMC, which was maintained in subsequent samples. Results of gDNA β-globin pyrosequencing were also compared to conventional STR-analysis. As summarized in Tables 2 and 3, both DNA-based methods revealed similar levels of donor chimerism. For Subject 1, both cytogenetic and Bioview FISH evaluation of PBMC and marrow for the presence of nucleated cells with the Y chromosome also revealed comparable levels of donor engraftment.

TABLE 3
Comparison of Hematopoietic Chimerism
Using Different Methods

| % Donor Post Transplant | | |
|---|---|---|
| Pre | 1 month | 3 months |

Subject 1

| | | | | |
|---|---|---|---|---|
| DNA | | | | |
| DNA pyrosequencing | 0 | 25 | 26 | |
| STR analysis | 0 | 24 | 27 | |
| XY cytogenetics | | | 25 | |
| XY FISH (Bioview) | | | 25 | |
| Cells: erythroid progenitors | 0 | — | 23 | |
| β-globin RNA | 0 | 69 | 66 | |
| | | | | Donor |
| % Hemoglobin S | 78 | 21 | 29.8 | 37 |

Subject 2

| | | | | |
|---|---|---|---|---|
| DNA | | | | |
| DNA pyrosequencing | 0 | 45 | 47 | |
| STR analysis | | 42 | 40 | |
| Cells: erythroid progenitors | 0 | 18 | 49 | |
| β-globin RNA | 0 | 97 | 100 | |
| | | | | Donor |
| % Hemoglobin S | 96.6 | 30.8 | 29 | 35.2 |

Measurement of Donor Engraftment by Cellular Analysis of Hematopoietic Precursor Cells

To directly measure the contribution of sex-mismatched donor cells to erythroid, granulocyte and lymphoid lineages in the reconstituted host, Bioview analysis was employed in Subject 1. In this method, FISH with X and Y-chromosome probes is performed in conjunction with morphologic analysis of stained bone marrow smears. For Subject 1, donor derived cells were found to constitute 25% of marrow erythroid precursors at 3 months post-transplant. Using the same technique, 25% of myeloid and lymphoid cells in the same marrow samples were also donor-derived. This method could not be utilized for Subject 2, who had received an allogenic transplant from a donor of the same sex. To directly quantify the number of donor erythroid precursors in this subject, erythroid cells were quantified through manual counting of MGG stained marrow cytopsin preparations and then compared to immunocytochemistry results for B antigen (donor-specific). While genomic chimerism of whole bone marrow derived mononuclear cells was approximately 40-50% one month after transplant, only 18% of erythroid lineage precursors were donor-derived post-transplant. By 3 months after transplant, 49% of erythroid precursor cells were donor-derived. Using genomic methods to assess PBMC chimerism, 47% of cells were donor-derived at this time. As summarized in Table 3, analysis of erythroid lineage chimerism using direct identification of donor erythroid precursors with either a Y chromosome probe or B antigen-specific antibody demonstrated levels of engraftment that were either similar to or less than that obtained with genomic chimerism analysis using either STR-based methods or gDNA pyrosequencing of the β-globin gene.

Measurement of Erythroid Lineage Chimerism by Hemoglobin Electrophoresis

To measure the contribution of donor cells to RBC in peripheral blood levels of hemoglobin S and A were monitored by hemoglobin electrophoresis. FIG. 5 and Table 3 summarize the results of hemoglobin electrophoresis during the first 3 months following transplantation for Subjects 1 and 2. Both subjects demonstrated a decrease in percent hemoglobin-S (Hgb S) from >78% to less than 35% following RBC exchange transfusion before transplant. The stem cell donors for both subjects had sickle trait, with 35-37% Hgb S and 62% Hgb A. In the early post-transplant period, Subject 1 received a total of 7 units pRBCs, and demonstrated a nadir of 22% Hgb S by day 80. By day 90, the level of Hgb S had risen to 30%. This remained lower than the level of Hgb S in her sickle trait donor, reflecting the continued contribution of transfused normal RBC. Subject 2 demonstrated a similar profile. His level of Hgb S was only 29% following transfusion of 2 units pRBC on day 31 and remained at this level (below the donor's level of 35% Hgb S) at day 90. Since both subjects received multiple RBC transfusions in the peri-transplant period, these studies confirmed that the results of hemoglobin electrophoresis did not accurately reflect the level of donor engraftment during this entire period.

Measurement of Erythroid Lineage Engraftment by β-Globin RNA Pyrosequencing

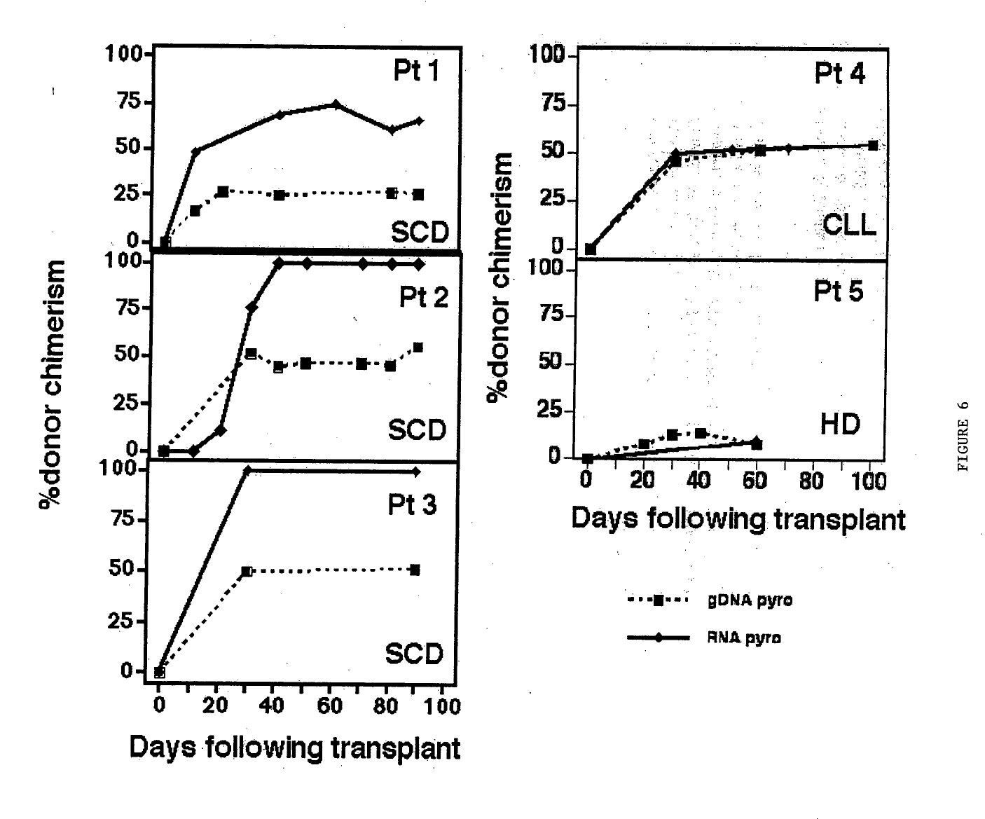

In both subjects, donor β-globin RNA was not detectable by pyrosequencing prior to transplant. As shown in FIG. 6, the level of donor β-globin RNA in Subject 1 rose to 48% by day 15, and fther increased to 69% by day 45 post transplant. This level of donor RNA expression was much higher than the level of donor DNA in marrow or peripheral blood or the number of erythroid precursor cells in marrow enumerated by FISH. By one month after transplant, Subject 2 had achieved 76% donor-derived β-globin RNA even though assessment of DNA only revealed 45% donor chimerism and analysis of marrow erythroid precursor cells only demonstrated 18% donor engraftment. At 2 months after transplant, DNA chimerism remained unchanged but donor-derived β-globin RNA expression had increased to 100% and remained at that high level. RNA pyrosequencing was performed on both marrow and peripheral blood samples of Subject 2, and yielded identical results.

Chimerism in a child with SCD (Subject 3) who underwent NMA allo-HSCT with an HLA-matched sibling donor with sickle trait was also measured. SCD subjects were compared to two subjects (Subjects 4 and 5) who underwent non-myeloablative allo-HSCT following fludarabine and busulfex conditioning for CLL and Hodgkin's disease, respectively. As shown in FIG. 6, all 3 SCD subjects engrafted with donor cells and had relatively stable levels of donor DNA chimerism between 30 and 100 days post transplant. During this period, Subject I had 20-30% donor DNA and Subjects 2 and 3 had 40-50% donor DNA. In all 3 SCD subjects, the level of donor β-globin RNA in peripheral blood measured by pyrosequencing was approximately 2 fold greater than the level of DNA chimerism. In contrast, the same analysis in subjects with hematologic malignancies showed equivalent levels of donor β-globin RNA and donor DNA chimerism. These experiments demonstrate that engraftment of relatively small numbers of normal donor hematopoietic stem cells in subjects with SCD can result in substantially higher levels of donor erythropoiesis in vivo. Since all three SCD subjects also had marked clinical improvement, these results suggest that as little as 25% donor engraftment can result in significant resolution of sickle cell disease symptoms.

β-Globin RNA Pyrosequencing Demonstrates Ineffective Host Erythropoiesis.

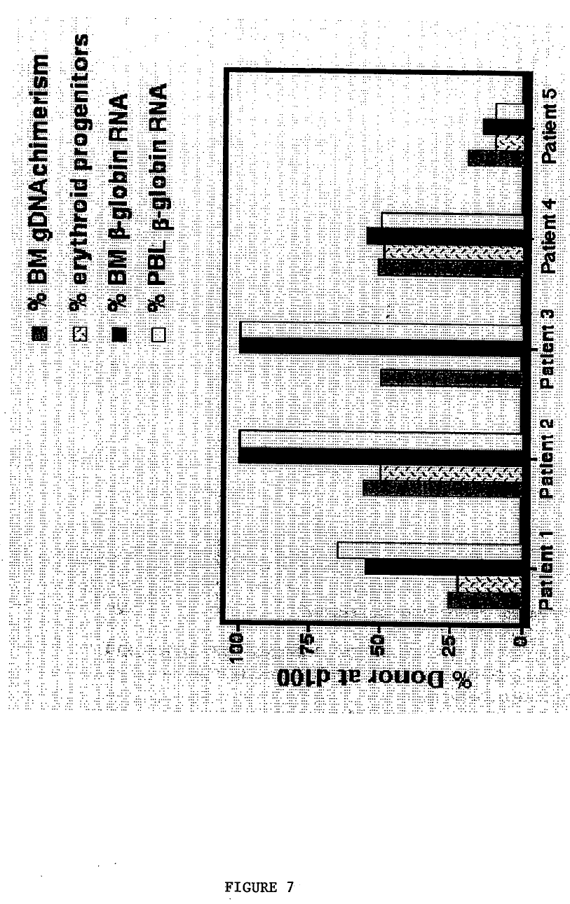

While the increased representation of donor erythropoiesis in peripheral blood likely reflects the rapid hemolysis of recipient RBC compared to RBC derived from normal donor precursor cells, analysis of β-globin RNA and gDNA in bone marrow cells also showed the same level of disparity. As shown in FIG. 7, Subject 1 demonstrated 55% donor β-globin RNA in marrow and 66% donor β-globin RNA in peripheral blood at the same time that donor marrow gDNA chimerism was only 25%. Similarly, Subjects 2 and 3 demonstrated 100% donor-derived β-globin RNA in marrow and peripheral blood, even though donor erythroid precursor cell engraftment was only 50%. This disparity between RNA and DNA chimerism was not seen in control subjects with hematologic malignancies (Subjects 4 and 5). This direct comparison of host and donor erythropoiesis in mixed chimeric marrow demonstrates a higher degree of ineffective host erythropoiesis than previously anticipated, and suggests that this mechanism of anemia is a more significant component of the pathophysiology of sickle cell anemia than previously appreciated. The presence of ineffective erythropoiesis in SCD explains the maturation advantage of AA or SA donor erythroid precursor cells over SS cells that allows for greater donor contribution to overall erythropoiesis following SCT. These findings support the notion that non-myeloablative HSCT can be curative for SCD in the setting of stable mixed hematopoietic chimerism.

Demonstration of Ineffective Erythropoiesis in Sickle Cell Disease.

Functional donor chimerism was also determined in purified marrow populations of immature (pro-and basophilic—glycophorin [GYPA+, CD71+hi]) versus mature (polychromatophilic, orthochromatophilic erythroblasts [GYPA+, CD71+dim]) cells to determine the stage of maturation at which SS erythroblasts are lost. While non-sickle cell disease subjects demonstrated similar rates of donor and host erythropoiesis, all sickle cell disease subjects (n=3) demonstrated progressive intramedullary loss of SS erythroblasts with maturation, with enrichment of donor erythroid precursor chimerism appearing from the earliest stages of hemoglobinization. The presence of ineffective erythropoiesis in sickle cell disease explains the maturation advantage of AA or SA donor erythroid precursor cells over SS cells that allows for greater donor contribution to overall erythropoiesis following SCT. This is the first definitive in vivo demonstration of ineffective erythropoiesis in sickle cell disease, and supports the notion that NMA-SCT can be curative for sickle cell disease in the setting of stable donor engraftment.

### Example 2

### Identification of Additional Allel-Specific Polymorphisms to Detect Lineage-Specific Cells

Additional polymorphisms specific to RBC lineage genes for measurement of RBC chimerism are useful for post-allograft erythroid-lineage specific chimerism and allow the monitoring of any subject-donor pair, regardless of the underlying disease. Some diseases in which knowledge of the extent of donor RBC engraftment would aid in the evaluation of the transplant procedure include MDS, aplastic anemia and thalassemia. Development of a RBC SNP panel for measurement of RBC chimerism also readily allows the evaluation of various clinical factors, such as ABO incompatibility, and the composition and intensity of the conditioning regimen, on RBC engraftment.

Based on public databases, such as Hembase, available through the National Institute of Diabetes and Digestive and Kidney Diseases (NIDDK), 13 constitutively expressed RBC lineage-specific genes have been identified, including genes associated with hemoglobin, the RBC cytoskeleton genes, and those which encode the RBC blood group antigens. Known SNPs present in the coding regions of these genes (i.e. exon or 3′UTR) were identified through publicly available databases, such as those available through GenBank. 3-58 candidate known exon or 3′UTR SNPs per gene (246 total SNPs) were found, as shown on Table 4.

TABLE 4
Analysis of allele frequency of RBC expressed SNPs.

| | Column number | | | | | | | |
|---|---|---|---|---|---|---|---|---|
| | (1) | | (3) | | | (6) | (7) | (8) |
| | # | | # SNPs | (4) | (5) | # SNPs | # SNPs | # SNPs |
| | candidate | | with >0.15 | # | # | with >0.15 | with >0.25 | with >0.35 |
| | SNPs in | (2) | frequency | informative | informative | frequency | frequency | frequency |
| | 3′UTR | # SNPS | in at least | SNPs in | SNPs in | for both | for both | for both |
| Gene name | or exon | tested | 1 population | Caucasians | Nigerians | races | races | races |
| Kell Blood group antigen | 37 | 25 | 10 | 0 | 10 | 0 | 0 | 0 |
| Lutheran blood group antigen | 8 | 4 | 4 | 4 | 1 | 1 | 1 | 1 |
| Glycophorin A | 18 | 16 | 14 | 7 | 9 | 1 | 1 | 3 |
| Glycophorin B | 58 | 34 | 2 | 2 | 2 | 0 | 0 | 2 |
| Glycophorin C | 11 | 11 | 9 | 9 | 9 | 2 | 2 | 5 |
| Rhesus blood group CcEe Ag | 18 | 9 | 6 | 4 | 5 | 0 | 0 | 1 |
| Rhesus blood group B | 8 | 8 | 5 | 5 | 4 | 1 | 2 | 1 |
| Solute carrier family 4, | 36 | 27 | 6 | 4 | 6 | 0 | 3 | 1 |
| Diego blood group | | | | | | | | |
| Solute carrier family, | 25 | 15 | 13 | 7 | 12 | 2 | 1 | 3 |
| Kidd blood group | | | | | | | | |
| Alpha globin 1 | 11 | 6 | 5 | 2 | 3 | 1 | 1 | 0 |
| Alpha globin 2 | 10 | 3 | 1 | 1 | 0 | 0 | 0 | 0 |
| Beta globin | 3 | 1 | 0 | 0 | 0 | 0 | 0 | 0 |
| Erythrocyte membrane | 3 | 3 | 2 | 0 | 2 | 0 | 0 | 0 |
| protein band 4.2 | | | | | | | | |

In order to develop a panel of expressed RBC SNPs that can be used for consistently identifying informative loci to distinguish between any subject-donor transplant pair, expressed RBC SNPs occurring at a high allele frequency across diverse populations were identified. The allele frequencies of candidate expressed RBC SNPs were determined, in a high-throughput fashion, in a reference panel of non-subject European- and African-derived de-identified, family-based samples. These samples comprise 96 independent European chromosomes and 124 independent Nigerian chromosomes. Genotyping of these samples to determine allele frequency within these populations was performed by Sequenom MassArray. This system is based on primer extension of multiplex PCR products with detection by matrix assisted laser desorption ionization-time of flight (MALDI-TOF) mass spectrometry, and is scalable due to considerable automation and software provided with the system. Enhancement of this system allows processing of 48×384-well plates per day, with a seven-plex reaction resulting in more than 120,000 genotypes per day; the automatic tracking of the large number of primer pools used each day; and automation of the addition of primer and probe directly to PCR plates.

PCR assays were designed for 165 candidate erythroid SNPs, representing 1-34 SNPs per erythroid-specific gene (column 2, Table 4), of which 77% were successfully genotyped. Assays were considered successful and genotype data were included in the analyses if they passed all of the following criteria: (1) >85% of all genotyping calls were obtained, (2) markers did not deviate from Hardy-Weinberg equilibrium, and (3) markers had no more than one Mendelian error. Informative expressed SNPs were defined as occurring at >0.15 population frequency. As shown in Table 5, 12 of the 13 genes were found to encode SNPs with >0.15 allele frequency in either the Caucasian or the Nigerian population (columns 4 and 5 of Table 4). Of the 165 SNPs, 76 occurred with greater than 0.15 frequency in at least one population (column 3 Table 4); 33 and 7 had at least 0.15 allele frequency in only the Nigerian and Caucasian populations, respectively. As shown in columns 6-8 (Table 4), 36, 28 and 17 expressed SNPs were found to occur in both Caucasian and Nigerian populations at a minimum population frequency of 0.15, 0.25 and 0.35, respectively.

TABLE 5

| Probability of at least | | | |
|---|---|---|---|
| one informative SNP | p > 0.15 | p > 0.25 | p > 0.35 |
| In a panel of 8 SNPs | 0.728 | 0.900 | 0.968 |
| In a panel of 12 SNPs | 0.858 | 0.968 | 0.994 |
| In a panel of 17 SNPs | 0.937 | 0.992 | 0.999 |

The criteria for inclusion of a SNP in the final expressed SNP chimerism panel is as follows: 1) amplicons from cDNA encoding the SNP of interest are readily PCR amplifiable. For each SNP, SNP-specific oligonucleotide primers that can amplify cDNA (rather than gDNA) are designed. The conditions of PCR amplification are optimized. In addition, SNP-specific sequencing primers, suitable for our pyrosequencing assay are designed and tested; 2) these SNPs are confirmed to occur at high allele frequency when amplified from cDNA derived from PBMC of at least 10-12 normal donors; 3) since quantitation of chimerism depends on calculation of area under the curve, allele-specific mRNA expression are not strongly biased toward particular gene alleles. This is readily determined by RNA pyrosequencing of a heterozygote, in which the observed pyrosequencing output should be similar to the predicted pattern and not biased; 4) confirmation that quantitative measures of allele-specific mRNA expression can be performed is determined by generation of a standard curve, following mixing of known amounts of RNA from 2 genotypically different individuals (as described above).

### Example 3

### Development of SNP-Based Assays to Monitor Engraftment of Distinct Hematopoietic Cell Types (Myeloid, T cell, B cell, NK Cell, Dendritic Cell) Following Allogeneic HSCT

To develop novel regimens that prevent delayed graft rejection in subjects with hemoglobinopathies treated with NMSCT, a better understanding of donor-host interactions leading to anti-donor sensitization and immune reconstitution following NMA allo-HSCT is required. Detailed kinetic characterization of host cell recovery and donor cell engraftment in blood and marrow following NMA allo-HSCT in man has been limited. Nevertheless, there have been several reports of preliminary associations between transplant outcome and extent of T cell and/or APC chimerism (Chan, G. W., et al. (2003) Biol Blood Marrow Transplant 9:170; Peggs, K. S., et al. (2004) Blood 103:1548; Koenecke, C., et al. (2003) Exp Hematol 31:911; Keil, F., et al. (2003) Transplantation 76:230; Dey, B. R., et al. (2003) Biol Blood Marrow Transplant 9:320). Most studies have focused on assessment of reconstitution of a limited number of immune cell lineages because: (1) isolation of multiple purified cell subpopulations is labor-intensive; and (2) cell material needed to perform the necessary manipulations to achieve multiple cell subset isolation is limiting, especially at the early time points following transplant. Since transplant outcome results from complex interactions between multiple donor and host cell populations, simultaneous analysis of multi-lineage subpopulations will required to comprehensively characterize hematopoietic chimerism and immune reconstitution. Although RNA pyrosequencing was originally developed to examine erythroid lineage chimerism, this method can be readily adapted to define chimerism of any cell subset, without requiring laborious cell isolation, provided that an informative expressed SNP on a gene that defines the cell subset of interest is utilized.

In order to assess post-transplant immune reconstitution, expressed SNP panels for B cells, T cells, natural killer (NK) cells, monocytes and dendritic cells (DCs) are developed. The source of cell subset specific SNPs is derived from known cell surface phenotypic markers that define these individual subsets. In order to expand the pool of candidate cell subset-specific genes, expression microarray datasets that were merged and normalized with publicly available data sets (both generated from the Affymetrix U95A platform (Chaussabel, D., et al. (2003) Blood 102:672; Chun, T. W., et al. (2003) Proc Natl Acad Sci USA 100:1908; Klein, U., et al. (2003) Proc Natl Acad Sci USA 100:2639), were analyzed. These datasets include microarray analysis of RNA expression in normal B, T, NK, macrophage and blood-derived dendritic cells. As shown in FIG. 7, in which genes in the merged dataset were clustered on the basis of RNA expression levels (darkest gray-high expression levels; lightest gray=low expression levels), each hematopoietic cell subset can be distinguished on the basis of expression of a defined set of genes.

Based on this microarray data as well as from known phenotypic markers that have been confirmed to be cell lineage specific based on literature review, a list of lineage specific genes has been compiled from which the expressed SNP panels are generated (Table 6). As shown in Table 6, 1 to 132 known expressed SNPs per gene (present in the exon or 3′UTR) have been identified through analysis of the publicly available dbSNP database (columns 2 and 3, Table 6).

TABLE 6

Column number

| | | | | (4) |
|---|---|---|---|---|
| | | | | # of |
| | | (2) | (3) | validated |
| | | # of | # of | or multiply |
| Cell | (1) | 3′UTR | exon | submitted |
| subset | Gene | SNPs | SNPs | SNPs |
| T cells | CD3E | 9 | 6 | 4 |
| | CD3D | 14 | 3 | 4 |
| | CD3Z | 19 | 4 | 18 |
| | CD3G | 11 | 2 | 5 |
| | TCRA | 19 | 0 | 7 |
| | LNK | 5 | 7 | 10 |
| | CD28 | 13 | 3 | 7 |
| B cells | CD19 | 6 | 1 | 2 |
| | CD20 | 11 | 2 | 5 |
| | CD22 | 17 | 6 | 10 |
| | CD79A | 9 | 1 | 3 |
| | CD79B | 32 | 15 | 19 |
| | B cell linker | 10 | 3 | 2 |
| Monocytes | CD14 | 23 | 7 | 18 |
| NK cells | CD56 | 18 | 5 | 13 |
| | CD94 | 26 | 5 | 26 |
| | CD16 | 91 | 41 | 52 |
| | CD160 | 9 | 2 | 5 |
| DCs | DC-IGN | 36 | 8 | 31 |
| | DC-AMP | 30 | 7 | 28 |
| | BDCA2 | 24 | 1 | 7 |
| | CD83 | 36 | 8 | 31 |

High density SNP microarrays (>10,000 SNP) are commercially 30 available, e.g., through Affymetrix. Many of the SNPs present on the commercially available arrays are non-expressed, but a subset are of interest are included in the present assay. Moreover, approximately 140 individuals of diverse racial backgrounds have been genotyped with these microarrays, and the data is publicly available. Therefore, the SNP candidates, tabulated in Table 6, first undergo cross-referencing with publicly available information to determine if population frequency data is already available. If not, then, allele frequency is determined by Sequenom MassArray as described above.

High allele frequency SNPs are identified (>0.15 frequency in both Caucasian and African populations), and the specificity and sensitivity of individual SNP candidates is important criteria to confirm a cell lineage specific expressed SNP-based assay. Therefore, for each SNP, mixing studies are performed between total PBMC RNA of 2 normal donors who are genotypically disparate at the SNP loci of interest. The series of RNA mixes are reverse transcribed into cDNA, PCR-amplified in a lineage-specific manner, and submitted for pyrosequencing. Pyrosequencing output is compared with known cell-subset specific chimerism, calculated based on prior immunophenotypic characterization of the PBMC. For example, to test a candidate B cell lineage-specific expressed SNP, (a) the number of B cells in PBMC derived from 2 independent normal leukopack donors who are genotypically disparate for the candidate SNP is calculated. This is performed by immunophenotyping PBMC with a CD20+ monoclonal antibody; (b) generate total RNA from these two sources of PBMC; and (c) perform mixing studies of the two total RNA samples. In order for the candidate SNP to be suitable for inclusion in the expressed SNP panel, the pyrosequencing outputs correlate well with the calculated number of lineage-specific cells within the inputted mixture, and consistently detect at least 5% donor cells. These studies are repeated with at least 5 donors per SNP to confirm reproducibility.

As shown in Table 6, while genes that are predominantly specific for a cell lineage subset have been selected, many have some degree of expression in other cell subsets as well (i.e., CD3 predominantly on T cells, but to a lesser degree on NK cells). The mixing studies delineate the extent that RNA pyrosequencing can sensitively and specifically reflect lineage-specific chimerism, and prioritize SNPs whose expression most closely matches chimerism based on cell number.

Since expression of some leukocyte genes may be up- or down-regulated in relation to cell activation state, similar mixing studies are performed wherein PBMC of one of 2 normal leukopack donors is activated (ie., addition of PMA and ionomycin for T cell activation; LPS for DC activation). Thus, the extent to which activation influences quantitation of chimerism is determined based on a specific lineage-associated SNP.

### Example 4

### Development of SNP-Based Methods to Monitor Engraftment of Functional Molecules Following Allogeneic HSCT

Example 3 describes the use of markers that are predominantly present on the cell surface to define a cell subset. However, various leukocyte populations can also be defined based on their functional activity, which is more biologically relevant to understanding transplant immunology. In response to activation or stimulation, a cell population may express activation markers, and secrete cytokines or chemokines, and signaling pathways are stimulated. A hallmark of T cell activation is secretion of cytokines that may be involved in proliferation (IL2, IL7, IL15), inflammation (IL1β, IL6, TNFα), tolerance induction (IL10, TGFβ, IL4) or cytolytic activity (granzyme B, perforin, IFNγ and granulysin expression). Likewise, DC activation is associated with the secretion of IL12; in peripheral blood, the primary source of IFNα secretion is from plasmacytoid dendritic cells (PD) (Ronnblom, L. and Alm, G. V. (2001) J Exp Med 194:F59). There is an association between cytokine secretion and transplant outcome. For example, GVHD has been associated with dysregulated proinflammatory cytokine expression (Ferrara, J. L., et al. (1996) Stem Cells 14:473). Additionally, murine studies indicate that the relative balance between secretion of TH1 and TH2 cytokines appears to contribute to determining the extent of GVHD (Krenger, W. and Ferrara, J. L. (1996) Immunol Res 15:50). Studies have also explored the functional activity of regulatory T cells (CD4+, CD25+) in engraftment and GVHD (Clark, F. J., et al. (2004) Blood 103:2410), and have implicated IL-10 in playing an important role in tolerance induction. Accordingly, expressed SNPs of functional molecules are used to determine whether effector activity of activated immune cell populations are host or donor derived. The use of expressed SNPs derived from functional molecules to measure chimerism will identify critical cell populations contributing to transplant outcome. For example, detection of primarily donor-derived proinflammatory and cytolytic cytokines indicates the initiation of GVHD. Conversely, detection of primarily host-derived immune cell activation indicates of incipient graft rejection. These studies are particularly useful for the analysis of immune reconstitution following NMA allo-HSCT, since coexisting donor and host derived hematopoiesis is frequently observed. Moreover, as alternative sources of allogeneic hematopoietic stem cells are increasingly used, these analyses provide information on the activity of specific cellular components derived from the stem cell source that may subsequently influence engraftment and the risk for toxicities (Rainsford, E. and Reen, D. J. (2002) Br J Haematol 116:702; Bofill, M., et al. (1994) J Immunol 152:5613).

Table 7 lists examples of cytokines and secreted factors that are associated with specific cell subsets and includes markers associated with general T cell activation, T/NK cell cytolytic activity, Th1 and Th2 activity, DC activation and tolerance induction. The number of known SNPs, recorded in the dbSNP database, expressed in the 3′UTR (column 2) and exons (column 3) of these genes are tabulated, as well as the number that have been identified by multiple sequencing efforts (column 4). Other functional molecules which contain allele specific variants useful in the methods of the invention include the chemokines and their receptors, and molecules associated with activation-induced signaling. As described above in Examples 2 and 3, a panel of SNPs is developed for each specific cell subset.

TABLE 7

| | Column number | | | |
|---|---|---|---|---|
| | | | | (4) |
| | | | | # of |
| | | (2) | (3) | validated |
| | | # of | # of | or multiply |
| | (1) | 3′UTR | exon | submitted |
| Cell subset | Gene | SNPs | SNPs | SNPs |
| Activated | IL2 | 9 | 3 | 4 |
| T cells | CD69 | 27 | 6 | 22 |
| | IL7 | 12 | — | 10 |
| | IL15 | 28 | 5 | 20 |
| Proinflammatory | IL1β | 30 | 7 | 22 |
| cytokines/ | TNFα | 56 | 9 | 25 |
| chemokines | IL6 | 24 | 6 | 21 |
| | IL8 | 15 | 11 | 16 |
| NK/T cell | Perforin | 9 | 5 | 6 |
| cytolytic | Granzyme B | 11 | 5 | 6 |
| activity | Granulysin | 17 | 4 | 11 |
| | IFNγ | 31 | 7 | 26 |
| T_{H}1 | IFNγ | 31 | 7 | 26 |
| | TNFα | 56 | 9 | 25 |
| | TNFβ | 90 | 10 | 47 |
| | GMCSF | 25 | 6 | 21 |
| T_{H}2 | IL4 | 12 | 3 | 11 |
| | IL10 | 19 | 10 | 14 |
| | IL13 | 43 | 6 | 33 |
| DC activation | IL12 | 13 | 8 | 16 |
| | IFNα (pDC) | 21 | 5 | 16 |
| Tolerance | IL10 | 19 | 10 | 14 |
| induction | TGFβ | 4 | 1 | 5 |

Validation of these SNPs is also performed as described above, and in addition, a variation of the mixing studies (also as described above) is used to confirm that the high allele frequency SNPs derived from functional molecules sensitively and specifically measure chimerism of the appropriate population. For example, to examine SNPs from the cytolytic panel, the well-defined model system of influenza A stimulation of HLA-A2+ cytotoxic T lymphocytes is utilized. Two previously influenza-vaccinated normal leukopack donors, one HLA-A2-positive and the other HLA-A2-negative, who are genotypically disparate for one of the candidate SNP is identified. PBMC from the two donors are stimulated overnight with the HLA-A2+ defined cognate peptide of the influenza matrix protein (M1: GILVATAAL), which will activate M1-specific cytotoxic T cells in the HLA-A2+, but not the A2-negative donor. The percent of perforin/granzyme B/IFNγ expressing cells per leukopack donor is quantified using conventional methods of intracellular cytokine staining of the functional molecule of interest. Following M1 peptide stimulation and flow cytometric analysis, RNA is extracted from PBMC of both donors, mixed in known quantities, reverse transcribed, PCRd and pyrosequenced. Pyrosequencing output is compared with the expected number of cells expressing the functional molecule of interest in the input mixture, as calculated from the known percent of positive-expressing cells detected by flow cytometry. Candidate SNPs are acceptable if they detect up to 5% donors cells.

### Example 5

### Development of a Panel of SNPs for High-Throughput Quantitative Monitoring of Lineage-Specific Engraftment Following Allogeneic HSCT

Utilizing the lineage-specific allelic variants identified and validated as described above, it is necessary to confirm that the assembled expressed SNP panels reproducibly yield informative SNP loci for each cell lineage when actual HLA-matched subject-donor pairs are examined. Furthermore, these confirmed sets of SNP panels are utilized to prospectively monitor the kinetics of multi-lineage donor engraftment in subjects enrolled in the clinical trials. The development of this high throughput tool provides the ability to assess lineage-specific engraftment in real-time. Development of these SNP-based chimerism panels is useful not only to investigate the impact of a novel conditioning regimen and hematopoietic stem cell source on post-transplant engraftment in hemoglobinopathy subjects, but these panels also have broad applicability for the monitoring of multi-lineage engraftment of any stem cell transplant population. These SNP-based chimerism panels also provide insight regarding the association of specific donor or host cell sub-populations on transplant outcome. These SNP panels are highly amenable to conversion to an oligonucleotide microarray format, which further facilitates high-throughput analysis, including automated analysis. In this format, one sample of total RNA from subject PBMC is sufficient for comprehensive analysis of chimerism across multiple cell lineages.

To confirm that the assembled expressed SNP panels reliably identify informative SNPs for each cell lineage in HLA-matched subject-donor pairs, high throughput genotyping is performed. This is first performed by pyrosequencing analysis of gDNA and then subsequently of cDNA. A lineage specific SNP panel is considered satisfactory if at least 1 informative SNP per cell lineage is identified for each subject-donor pair. For example, if it is assumed that each lineage panel comprises 12 SNPs, then cDNA from 8 subjects can be genotyped per lineage in a 96 well format. If less than 1 informative SNP per cell lineage is identified per subject-donor pair, then additional candidate SNPs are evaluated and added to the panel, as described above.

If SNP typing of gDNA is satisfactory, SNP frequency in cDNA samples is assessed. Following reverse transcription of gDNA into cDNA from matched related subject donor pairs, including subjects of Afro-American descent, SNP frequency is assessed in these transplant pairs by pyrosequencing. A lineage specific SNP panel is satisfactory if at least 1 informative SNP per cell lineage is identified for each subject-donor pair.

Once the completed and validated lineage-specific expressed SNP panels are created, they are utilized to prospectively monitor lineage-specific engraftment for subjects. The SNP-based multi-lineage chimerism analysis requires a three-stage process: (1) genotyping of each subject and donor pair (utilizing gDNA derived from donor and pre-transplant subject PBMC) to identify the informative SNP loci per each cell lineage/functional molecule-specific SNP panel; (2) PCR amplification of cDNA (generated from reverse transcription of total RNA) using the identified cell lineage-specific primers to generate the amplicon from which the informative SNP will be assayed. 2-4 informative SNPs are used to track engraftment of each lineage; (3) pyrosequencing using SNP-specific sequencing probes to directly quantify chimerism.

For these analyses, total RNA is extracted from PBMC prospectively collected at 2, 4, and 6 weeks, and at 3, 6, 9 and 12 months following transplant to determine the kinetics of reconstitution. Data generated from the SNP-based chimerism studies is analyzed in conjunction with immunophenotyping and T cell reconstitution studies, as well as with overall gDNA chimerism measured by conventional STR analysis. These data provide a description of the rate of cellular reconstitution following NM SCT.

A critical question regarding the efficacy of NMA-HSCT as a curative therapy for treatment of hemoglobinopathies is whether end-organ damage can be prevented or even improved. Since mixed hematopoietic chimerism is a frequent consequence of NMA-HSCT, an unanswered question is how much donor chimerism is sufficient for functional end-organ improvement. Improvement in end-organ damage can result from either prevention of ongoing damage from the sequelae of sickling and hemolysis, or alternatively, from regeneration of damaged tissue by donor derived non-hematopoietic cells, presumably arising from donor mesenchymal or multi-potent hematopoietic stem cells.

Subjects having sickle cell disease experience chronic intravascular hemolysis on endothelial function and end-organ injury. Also, pulmonary hypertension is a common end-organ complication of SCD, with an incidence of 20-40% in the adult population, and is associated with a 2-year mortality of 50% (Castro, O., et al. (2003) Blood 101: 1257; Gladwin, M. T., et al. (2004) N Engl J Med 350:886). Serum biomarkers of intravascular hemolysis have been identified that are strongly correlated with endothelial damage and pulmonary hypertension. These include VCAM-1, increased NO consumption and increased cell free hemoglobin (Reiter, C. D., et al. (2002) Nat Med 8:1383). Accordingly, it is determined whether mixed chimerism following NMA-HSCT is associated with improvement in multiple parameters of chronic intravascular hemolysis.

The transplantation of bone marrow can result in the generation of adipocytes, cardiomyocytes, hepatocytes, osteoblasts, renal mesangial cells, endothelial cells and chondrocytes, with donor engraftment ranging from 3-26% depending on the tissue type and model system (Asahara, T., et al. (1997) Science 275:964; Ito, T., et al. (2001) J Am Soc Nephrol 12:2625; Imasawa, T., et al. (2001) J Am Soc Nephrol 12:1401; Grant, M. B., et al. (2002) Nat Med 8:607; Grigoriadis, A. E., et al. (1988) J Cell Biol 106:2139; Lagasse, E., et al. (2000) Nat Med 6:1229; Makino, S., et al. (1999) J Clin Invest 103:697; Murohara, T. (2001) Trends Cardiovasc Med 11:303; Peichev, M., et al. (2000) Blood 95:952; Pittenger, M. F., et al. (1999) Science 284:143; Schatteman, G. C. and Awad, O. (2004) Anat Rec 276A: 13). The mechanism by which this occurs has been controversial, and various studies have supported one or more of the following hypotheses: (1) the existence of a pluripotent stem cell that is capable of maturing into different cell lineages; (2) the presence of transdifferentiation, in which bone marrow cells are genetically reprogrammed when exposed to a different microenvironment; (3) cell fusion occurs (Ying, Q. L., et al. (2002) Nature 416:545; Terada, N., et al. (2002) Nature 416:542), in which bone marrow cells mature cells fuse with cells of diverse tissue phenotypes; and (4) Bone marrow cells are a source of specific stem cells present in different tissue (Grove, J. E., et al. (2004) Stem Cells 22:487). Regardless of the underlying mechanism by which donor-derived marrow cells arrive in non-hematopoietic tissue, a consistent finding in these studies has been that donor cells appear to be attracted to sites of injury and tissue ischemia, perhaps due to increased rates of cellular incorporation or by specific injury-induced homing of progenitors (Ortiz, L. A., et al. (2003) Proc Natl Acad Sci USA 100:8407). In adult subjects with hemoglobinopathy, endothelial and end-organ injury are commonly observed, and thus allogeneic HSCT may result in significant incorporation of donor-derived cell in non-hematopoietic organs. Therefore, if post-transplant end-organ functional improvement is observed, it is necessary to determine whether functional improvement can be attributed simply to the benefits of donor erythropoiesis alone, or to actual normal donor cell contribution to non-hematopoietic tissue. Thus far, the only available methods for monitoring the fates of marrow-derived donor cells into non-hematopoietic tissue (primarily in model systems) have been by labeling donor cells with green fluorescent protein, or identifying donor Y chromosome in female recipients. These assays are not cell-lineage specific, and the use of secondary criteria is required, such as morphology or co-localization with specific markers to establish cell identity. The RNA-based expressed SNP approach described herein is utilized to examine non-hematopoietic lineage chimerism by choosing the appropriate cell-lineage specific SNP, and thus advance the ability to evaluate the impact of a stem cell therapy.

Accordingly, SNP based assays for measurement of, for example, endothelial, stromal and osteoblast lineage donor engraftment are described herein. Progenitor cells have been transplanted for the treatment of non-hematologic illness, such as osteogenesis imperfecta and myocardial injury syndromes (Assmus, B., et al. (2002) Circulation 106:3009; Strauer, B. E., et al. (2002) Circulation 106:1913; Horwitz, E. M., et al. (1999) Nat Med 5:309) and neurological and cognitive diseases and disorders, such as Alzheimer's disease and Parkinson's disease. Tracking donor engraftment in relevant non-hematopoietic tissue provides a method to quantify the impact of therapy.

Since endothelial cells are central to SCD pathogenesis, an expressed SNP-based assay to monitor endothelial lineage specific chimerism has been developed. Through literature review and extensive analysis of public databases, a series of endothelial cell specific genes (shown on Table 8) has been identified, and using the same experimental approaches as described above, a series of potentially informative SNP candidates have been identified. As seen in Table 8, of the 84 SNP candidates derived from 3 endothelial cell-specific genes (column 1), 62 were assayed (column 2), and 14 were found to occur at high allele frequency in both Caucasians and Africans (columns 6-8). Other endothelial cell specific genes from which potentially informative expressed SNPs are identified include VE-Cadherin, VEGFR1, VEGFR2, and tie-2, an endothelial-specific tyrosine kinase. SNPs are validated using the methods described above. Specifically, pyrosequencing output of mixes of total RNA extracted from PBMC of normal donors who are genotypically disparate for the SNPs of interest, are compared with known cell chimerism calculated from prior immunophenotyping with anti-CD146 antibody, which detects circulating endothelial cells.

TABLE 8
Analysis of allele frequency of expressed SNPs of endothelial cell origin.

| | | | Number | | | |
|---|---|---|---|---|---|---|
| | | | of SNPs | | | |
| | Number of | | detected to | Number | Number | Number |
| | candidate | | be frequently | of SNPs | of SNPs | of SNPs |
| | SNPs in | Number | polymorphic | informative | informative | informative |
| | 3′UTR or | of SNPS | in at least | in Caucasian | in Nigerian | for both |
| Gene name | exon | tested | 1 population | population | population | races |
| Vascular cell adhesion molecule 1 isoform a | 28 | 14 | 6 | 2 | 6 | 2 |
| (V CAM1) | | | | | | |
| Nitric oxide synthase 3 (NOS3) | 32 | 30 | 5 | 4 | 3 | 2 |
| Von willebrandt factor precursor (V WF) | 24 | 18 | 17 | 13 | 12 | 8 |
| Heme oxygenase 1 (HMOX1) | 27 | 21 | 12 | 10 | 10 | 8 |
| Heme oxygenase 2 (HMOX2) | 6 | 6 | 5 | 5 | 5 | 5 |

SNP based chimerism assays specific for stromal cells (i.e., candidate genes include fibronectin, vimentin, smooth-muscle actin, N-cadherin) and osteoblasts (i.e., candidate genes include type 1 procollagen, alkaline phosphatase, osteocalcin) are also similarly developed. Panels of expressed endothelial, stromal and osteoblast SNPs are assembled, and their reliability for identifying informative SNP between any HLA-matched subject-donor pairs is tested on paired samples of gDNA, and then cDNA samples, as described above.

Post-transplant endothelial, stromal cell and osteoblast chimerism is monitored by tracking donor chimerism by RNA pyrosequencing. Since bone marrow aspirate contains cells of endothelial, stromal and osteoblast lineages in addition to hematopoietic lineage, total RNA is extracted from post transplant marrow aspirates samples and used as starting material for the SNP-based chimerism assays. Marrow aspirates is collected at 1, 3, 6, and 12 months after transplant.

TABLE 9

| | | | |
|---|---|---|---|
| | GeneBank | | |
| | Accession | | SEQ ID |
| Gene | No. | SNP Sequence | NO. |

Kell Blood Group
Antigen

| | | | |
|---|---|---|---|
| * | rs8176039 | CCTTCAATGACTCCCTCACATTCTT | 204 |
| | | [A/G]GAGAATGCTGCAGACGTTGGGG | |
| | | GGC | |
| * | rs4252393 | TTGCATCTAAGGGTATAATGCAGAA | 205 |
| | | [C/G]AAAGAGGCACCCTGTAACTCA | |
| | | CCCC | |
| * | rs4252391 | TGGAGAGGCTCCAGCTTTATCTCTT | 206 |
| | | [G/T]CTGGGTTCAAATCTGGGTATGG | |
| | | TAG | |
| * | rs4252388 | GGGGACCTCAGAGGAGCCACTCATC | 207 |
| | | [A/G]GCCTGTCCTTTCTGTTCTTCCCC | |
| | | CT | |
| * | rs4252381 | CTCTGAGAAAGTGAGCTTCCAGCCC | 208 |
| | | [G/T]AACTTGGACCCTCTGCAGAGGG | |
| | | GCA | |
| * | rs4252372 | GACATGGGGGGTTTTCTACCTAAGG | 209 |
| | | [C/T]AGAAGGGCCCGGGAGCCAACTC | |
| | | CAG | |
| * | rs4252365 | CAGGTACCAGAGCCTGCTCGGCGAC | 210 |
| | | [C/T]CTCCAGGAAGCTGGCCGGTCAC | |
| | | GGT | |
| * | rs4252357 | AGTCACCTTTTATCCTCAGGGGATA | 211 |
| | | [C/T]ATTCCAAGACGCCCCAGTGGAT | |
| | | GCC | |
| * | rs6943031 | CTGCAGGTAGGTGGAAAAAAAGAC | 212 |
| | | A[A/G]GAGCCATACAGAGAAAACAC | |
| | | CCTCT | |
| * | rs8176049 | GTTGGAGACGAGAAGGGCTTGGATT | 213 |
| | | [A/C]AGTATCGGAAAGCTGCTCCCA | |
| | | ATTG | |
| Alpha Globin 1 | | | |
| # | rs2858016 | CCGGGGGCATGAAGCTCAGTTTGGT | 214 |
| | | [A/G]AGCACCACCTGGCAGGTGTAC | |
| | | CAAA | |
| * | rs3888374 | GGGCGGCGTTAAGGGCCCGGGGCTG | 215 |
| | | [A/G]CTCGGAGCAGGTTAGGGAACA | |
| | | GCGC | |
| * | rs4505325 | GCCTCCGCATCCTCCTCTCCTCCCC | 216 |
| | | [C/T]CAGGACTACCCTTAACCCACTC | |
| | | AGG | |
| * | rs4510012 | CCCATGCGCTCCCCAAGCCCAAGGT | 217 |
| | | [A/G]ACCCTGTGCAGGAGGCAAAGGT | |
| | | GGT | |
| # | rs2685118 | TCCTTCAGCTGTAATCAGCAGACAC | 218 |
| | | [A/G]GGGGACAGGATGGCCAGGGGC | |
| | | ACAG | |
| Alpha Globin 2 | | | |
| % | rs2685121 | GCCTGTGTGTGCCTGGGTTCTCTCT | 219 |
| | | [A/G]TCCCGGAATGTGCCAACAATGG | |
| | | AGG | |
| Heme Oxygenase | | | |
| 1 | | | |
| # | rs1807714 | GAACTCCTGACCTCGTGATCCACCT | 220 |
| | | [A/G]CCTCGGCCTCCCAAAGTGCTGG | |
| | | GAT | |
| # | rs2018898 | CGGAGGTTGCAGTGAGCTGAAATTA | 221 |
| | | [C/T]GCCACTGCACTCCAGCCTGGGT | |
| | | GAT | |
| # | rs4645726 | GTTAGGGTGCGAGAACCCGGGTCGG | 222 |
| | | [C/G]GACGCGGGTGAGCCGCGCGGT | |
| | | AATC | |
| # | rs4239877 | GGGAAGCGAATACTCCACAGGGCA | 223 |
| | | C[A/G]GTCTCCCAATGCAAAATGGG | |
| | | CACCT | |
| % | rs743813 | AGTTCCAGTGTGGCCGCTCACACGC | 224 |
| | | [C/G]TCTACCAGCCTGCTAGAGTCCT | |
| | | GGA | |
| # | rs743815 | ATAAATAAATGAATGAGCGAAGTG | 225 |
| | | A[C/T]GTGAATCCTAACTGCCATCTT | |
| | | CACC | |
| # | rs4645742 | GTGAAAGGACTTAGGGAAGTTTTCC | 226 |
| | | [G/T]GGGGGCTGAAGTTTGAGGGGA | |
| | | CCTT | |
| % | rs737777 | ACTGCGAGGCTGGGGAACAGCCTGT | 227 |
| | | [G/T]CGTTGGTCAGTGGTCCCCAAAT | |
| | | ATG | |
| * | rs4645743 | CTGCGAGGCTGGGGAACAGCCTGTG | 228 |
| | | [C/T]GTTGGTCAGTGGTCCCCAAATA | |
| | | TGT | |
| * | rs4645746 | TTTCCAAGAGAAGTTAAAGGCTCTG | 229 |
| | | [C/T]GGAGTCCTTCAGTGACAGAACT | |
| | | GGT | |
| # | rs713618 | CAGCATTCGTAGCCTGAAGGTGGGT | 230 |
| | | [C/T]GGAGGGCAGTGGCTGCTGCAT | |
| | | GGTG | |
| # | rs713873 | GCCAGACCTGAGTGCTAGCTGTGTG | 231 |
| | | [A/G]CCTTGAGTGAGGCACTTGACCT | |
| | | TTC | |
| VCAM1 | | | |
| * | rs3783608 | TTTCTATTTCACTCCGCGGTATCTG | 232 |
| | | [C/T]ATCGGGCCTCACTGGCTTCAGG | |
| | | AGC | |
| * | rs3783609 | TACCCTCCCAGGCACACACAGGTGG | 233 |
| | | [A/G]ACACAAATAAGGGTTTTGGAA | |
| | | CCAC | |
| * | rs3783613 | AGAGATCCAGAAATCGAGATGAGT | 234 |
| | | G[C/G]TGGCCTCGTGAATGGGAGCTC | |
| | | TGTC | |
| # | rs3176878 | TTCCAGGAAGAGAAAACAACAAAG | 235 |
| | | A[C/T]TATTTTTCTCCTGAGCTTCTCG | |
| | | TGC | |
| * | rs3176879 | CATATAGTCTTGTAGAAGCACAGAA | 236 |
| | | [A/G]TCAAAAGTGTAGCTAATGCTTG | |
| | | ATA | |
| # | rs3181092 | ATTATAAACTGCCTCCTTTAGTCAC | 237 |
| | | [A/G]TTGTAGCTCTTTCTGAAGTGCTA | |
| | | AG | |
| Erythrocyte mem- | | | |
| brane protein | | | |
| band 4.2 | | | |
| * | rs1474199 | GATATAAGTAAGACCTGAGTTTTGT | 238 |
| | | [A/G]TTAGCATGTAGGTTAAAGCATG | |
| | | TGG | |
| * | rs495286 | AAAAAAAAAAAAAAAAAAAAAGGC | 239 |
| | | G[A/G]GGCCTATTATTATACTTTTAC | |
| | | CACG | |
| Glycophorin A | | | |
| * | rs4524333 | TGTAACTCTTTGTGACTGAAGAAGA | 240 |
| | | [A/G]GTTGAAGTGTGCATTGCCACCT | |
| | | CAG | |
| # | rs4449373 | ACTTAATGCTGATATGCTCACAATT | 241 |
| | | [G/T]CTGTATAAAATAGAAGTTGAGA | |
| | | AAG | |
| % | rs1132787 | ATAGTTAAATTTGGTATTCGTGGGG | 242 |
| | | [C/T]AAGAAATGACCATTTCCCTTGT | |
| | | ATT | |
| # | rs6537251 | TAATGGGCTTTACATTTTAAACATA | 243 |
| | | [A/G]CTTGAAATAACAAATCATGACT | |
| | | ATA | |
| * | rs4643761 | TGGGTAGTTTATAAAGGAAATAGGT | 244 |
| | | [G/T]TAATTGACTTACTGTTCCCCAT | |
| | | GGC | |
| # | rs6537252 | ATGCTCTAGAAGCTAACACTCACAC | 245 |
| | | [A/T]GGCAGTCTTGGTGTTTTCCAGGT | |
| | | AG | |
| * | rs6537253 | AAACTGGTCTATGACATAATCACTT | 246 |
| | | [C/T]GGTGGACTCTATTCTAGACACA | |
| | | GTG | |
| # | rs6844670 | GGGTCAGAGAATGTGGCAACTTATA | 247 |
| | | [A/G]GTTCTATATCTTTTGCTCAGAT | |
| | | GGT | |
| * | rs4557214 | ATCTTGTGAGAACTCACTCACTATT | 248 |
| | | [A/G]TGAGAACAGCATGAGCAGCATG | |
| | | AGG | |
| * | rs4130880 | GCTGGAGGGTTTAGAAACTTTGAGT | 249 |
| | | [A/G]GCAAGATGGAAGAAAAGGAAT | |
| | | AAAT | |
| * | rs4535287 | TTCCTGGCCTGGCTGTGATACCTAT | 250 |
| | | [A/G]CACAAGTATTTTACACCATTCTT | |
| | | AC | |
| % | rs4350961 | GTTGTGTCTTTTGCAGACAAGTAAT | 251 |
| | | [A/G]TAGTATCTTGCAGGTAAACTTA | |
| | | TAA | |
| * | rs4613516 | GCATTTGTGCGCTTCCTCCTTATGG | 252 |
| | | [A/T]TTAACCTTGGTTTAGTTTCTATG | |
| | | CA | |
| # | rs4469024 | CCTCATTTAGATCTTTTGCTCATAA | 253 |
| | | [C/T]GACACAAGCAAACAACCAGGGC | |
| | | TGA | |
| Glycophorin B | | | |
| # | rs1511423 | GGTTTTATAAAAGTAACTTTATTTT | 254 |
| | | [C/T]AGCTTGTCTGTAGATTCAATTTT | |
| | | CT | |
| # | rs1511427 | CTTGTACAGACTAGAGGGTCACATT | 255 |
| | | [A/G]TGTAAAGCCTAACATAGCCCTG | |
| | | CAA | |
| Glycophorin C | | | |
| # | rs6568 | AGCTCAGAACGATTGGAAATAAATT | 256 |
| | | [G/T]GAAATGTAACCGAGCATTCCG | |
| | | AGTC | |
| # | rs935024 | GGCCAGGACAGTTCATTTCTGGCTC | 257 |
| | | [C/T]TGTGGGGGCCAGTTCCCAGAGA | |
| | | GCA | |
| # | rs3791345 | GACATTTCACCCTTCCTTTTGTTTC | 258 |
| | | [G/T]TGCAGCTGGTGCCCCTGTAGGGA | |
| | | AA | |
| # | rs1530149 | CCAAAGATCTCCCTGGGAGGATTAC | 259 |
| | | [A/G]GGGAATGTGAGCTCAGTGGGG | |
| | | GGTC | |
| # | rs6431172 | GATGACATGGAAGCTGCAGAGCTGG | 260 |
| | | [G/T]TTGTCCCAGGTGGCCTAGCTCA | |
| | | GAT | |
| # | rs6431173 | CCAGGTGGCCTAGCTCAGATGCCAC | 261 |
| | | [A/G]AGGAGGGAACTGTTGCAGGCT | |
| | | AACT | |
| # | rs6431174 | CCAGATGGCCACACCAGAGAGCAG | 262 |
| | | G[A/G]CAACTTAAAGAGATAATGCA | |
| | | CACAC | |
| # | rs1000857 | CAAATACAAAAAAAAAGCCACCTTT | 263 |
| | | [A/T]ATTTTTCAAGTAGCTGAAATTA | |
| | | TTA | |
| # | rs1000858 | TTCCTCTGGGCTTACTGAGATTCCC | 264 |
| | | [C/T]AAGTCAGAGTGAGGCCACTCCT | |
| | | TTC | |
| Rhesus Blood | | | |
| Group B | | | |
| # | rs2245623 | ACGCTGCAGGAAGACCATGAGGAA | 265 |
| | | G[C/T]CAAAGCCCACGAAGACCATG | |
| | | GCATG | |
| # | rs3748569 | TTGTACCCCAGCGTGGAGACAGTCC | 266 |
| | | [A/G]AGCCAAGAAGCCAGCTGCCAG | |
| | | AGCC | |
| # | rs4661163 | TTGCCACTTATGTCCCTGTCCTGAG | 267 |
| | | [A/T]CTGAGGAGCGGGGAATAGCAA | |
| | | GGGC | |
| % | rs6427311 | GATAGTTCCAAGCTCTATCACCTCC | 268 |
| | | [A/G]TGAAGCCTCCCATAGCCCAGGC | |
| | | AAT | |
| # | rs4661165 | GAGAAAAAAAGTCCTGAAAACAAA | 269 |
| | | A[A/C]CCCGACAACAACCTTAAAGT | |
| | | AGGAG | |
| von Willebrandt | | | |
| Factor Precursor | | | |
| # | rs216902 | GCCGAGTGGAGCTGCCTGTGCACAC | 270 |
| | | [A/G]CCTGGACAGAGAGAAGCAGAG | |
| | | GATG | |
| % | rs216310 | GTGCCTCGCTGAAGGGGTACTCCAC | 271 |
| | | [A/G]GTCACCATGTAGGAGTACTGCA | |
| | | GCA | |
| % | rs216311 | CGACCGCCCTGAAGCCTCCCGCATC | 272 |
| | | [A/G]CCCTGCTCCTGATGGCCAGCCA | |
| | | GGA | |
| % | rs216321 | GTCTGTGCAGTTCCACTTCCGGTCC | 273 |
| | | [C/T]GACAGACACTAGGAGCAGTCAT | |
| | | GGC | |
| # | rs1063857 | AGTGTACCAAAACGTGCCAGAACTA | 274 |
| | | [C/T]GACCTGGAGTGCATGAGCATG | |
| | | GGCT | |
| # | rs1800378 | GTTCCAGGTGACCTCCGCATCCAGC | 275 |
| | | [C/T]TACAGTGACGGCCTCCGTGCGC | |
| | | CTC | |
| * | rs1800376 | GGGAGTGCCTTGTCACAGGTCAATC | 276 |
| | | [G/T]CACTTCAAGAGCTTTGACAACA | |
| | | GAT | |
| * | rs1800375 | TCTCTCCAGGGGAGTGCCTTGTCAC | 277 |
| | | [A/T]GGTCAATCACACTTCAAGAGCT | |
| | | TTG | |
| # | rs2362481 | ACATTTATCTCCCCAAGTAGGCTCT | 278 |
| | | [C/G]AGATCCAGTTCAGCACCCAGGA | |
| | | CAA | |
| # | rs6489676 | GAACTGGCAA[A/C]TGGATAAACAA | 279 |
| | | AATGTAGCCTATCC | |
| # | rs6489677 | GGATAAACAAAATGTAGCCTATCCA | 280 |
| | | [C/T]CCAAGGAAATAGATTTCAGCTA | |
| | | TAA | |
| * | rs6489679 | CTGCTGCAAGAATGGCTATAACTAA | 281 |
| | | [A/G]AAGTCAAAAATTTTACTAGATG | |
| | | TCG | |
| % | rs6416321 | CCCTTGGGGGTCCCCAATCCCGAAG | 282 |
| | | [A/G]GTGGGAGTTCGGATGCCGCCTC | |
| | | CTT | |
| * | rs6489681 | ACCTGCTTCGAGTGTAAAAATTCAG | 283 |
| | | [C/T]CGGATTATCTCATGAACTGTCTG | |
| | | GC | |
| # | rs6416322 | GAGGCAGGAGGAGGGGTCCATTTTA | 284 |
| | | [C/T]AAGAGCTCTCCCATGAGACGCT | |
| | | AGG | |
| # | rs6489682 | TCCCCAGAGGGGAAGAGGAGTGCC | 285 |
| | | A[G/T]CTCCCCGGGACAGTTCACGGA | |
| | | CCTG | |
| % | rs1990326 | GGCAAAGACATCACTCTTCCCCAGA | 286 |
| | | [A/G]GTTGGCAGTATCTCACACTGAC | |
| | | ACT | |
| Nitric Oxide | | | |
| Synthase 3 | | | |
| % | rs1799983 | CCCTGCTGCTGCAGGCCCCAGATGA | 287 |
| | | [A/C]CCCCCAGAACTCTTCCTTCTGCC | |
| | | CC | |
| * | rs3918211 | GCCACATGTTTGTCTGCGGCGATGT | 288 |
| | | [C/T]ACCATGGCAACCAACGTCCTGC | |
| | | AGA | |
| % | rs3800787 | CAGAGCTGGAACATAACATGAAGC | 289 |
| | | A[C/G]GTCAAAAGTCATGCCCTCCTC | |
| | | CCGC | |
| # | rs1835428 | GCACAAGCAGCGCGGCGAAGAGTG | 290 |
| | | C[A/G]CCCGCGAAGAAAACGAGCTG | |
| | | GCGGG | |
| # | rs6464119 | TGGGAGAATCTCTCCTTCCCATTTC | 291 |
| | | [C/T]AGTGCACCCACCAACTCTGGAA | |
| | | CAG | |
| Rhesus Blood | | | |
| Group CcEe Ag | | | |
| * | rs3093621 | TTCACCTTGAGGGACCCTAGAAAAA | 292 |
| | | [C/T]AGAAAACCCATTTAATGGTCCA | |
| | | GGT | |
| * | rs3093629 | TCCAGGTGTTTAGGCCAAAAGCCCT | 293 |
| | | [A/G]GATGGTGAAGGCCTGTCAAAGA | |
| | | TGA | |
| * | rs3093639 | TGAAGCCTTAAGGACAACTGTAGAC | 294 |
| | | [A/G]TTCCCTCACCGAAACCCTAGAC | |
| | | CAG | |
| % | rs3093653 | TATGCCCCCAGTAAATATTTGTTAA | 295 |
| | | [A/G]TGAACTAAGGGAGCTATCATTT | |
| | | ACT | |
| # | rs1053438 | ATGCACAGTCACTCCACATCCACCA | 296 |
| | | [C/T]TGAAGGAAAGGAAAAAAGGGC | |
| | | AGAG | |
| # | rs9689 | GCCATGGTCATCTGACAGTCTCTAC | 297 |
| | | [A/G]CTGTGAATATTGCCTGGTGATC | |
| | | AAG | |
| Lutheran Blood | | | |
| Group Antigen | | | |
| # | rs7026 | GAGCTATTTTTACCTCCCGCCTCCC | 298 |
| | | [A/G]TGCTGGTCCCCCCACCTGACGT | |
| | | CTT | |
| # | rs8113311 | GAGTCCCTGGGGAAAGCGGGGAGC | 299 |
| | | T[G/T]GGAGGAATCCAGAGAAAAGG | |
| | | GGGCC | |
| # | rs1871045 | CCCAGGCAGAAGAGCTCATGGGCCA | 300 |
| | | [C/T]GTTAAGGCGCTCAGATCCCATG | |
| | | GTT | |
| % | rs4803759 | ATGTTGCCCAGGCTCGTCTTGAACT | 301 |
| | | [C/T]GAGCTCAAAATGATCTGCTTGC | |
| | | CTT | |
| Heme Oxygenase | | | |
| 2 | | | |
| # | rs1051308 | TCAGCCCCAGCTTATCTCCTCCTCC | 302 |
| | | [A/G]CGCTGTGTAAATGCTCCAGCAC | |
| | | TCA | |
| # | rs7702 | GCCTGTCAGCCTCAGCCCAGTCTGT | 303 |
| | | [C/G]TTGCTTAAGGGTGCGCCTCCCC | |
| | | AGG | |
| # | rs8129 | AGACCAACAACACACAAGCTCATTG | 304 |
| | | [C/T]CAGCCCCCTGCCACCCACTGAC | |
| | | CCT | |
| # | rs7665 | GGGAGCGGGAAAGTGACCACTGAG | 305 |
| | | C[A/G]CAGGGAGCAAAGCACAGGGG | |
| | | GCCAG | |
| # | rs4786509 | GTGTAATAAAAACTACAAGAATAAT | 306 |
| | | [C/G]TTTCAATCACACGATCAGAAGC | |
| | | AAA | |
| Solute carrier | | | |
| family 4, Diego | | | |
| blood group | | | |
| * | rs3819179 | GCAGTGGCTGATAAAGATTTGCCCA | 307 |
| | | [C/T]CTCCACGACAAGGGAGAGTGC | |
| | | TTCT | |
| * | rs2298720 | ATACCTTTAAGCTGGTTGGCAAGTT | 308 |
| | | [A/G]TTTCATGTCACCGGTGACATAG | |
| | | CCA | |
| # | rs2298718 | GTATGACCAGTTTGGCTGGAAAGAA | 309 |
| | | [A/G]GGATTGTAATGTCCTGTGGCTG | |
| | | AAA | |
| # | rs1058396 | ACTCAGTCTTTCAGCCCCATTTGAG | 310 |
| | | [A/G]ACATCTACTTTGGACTCTGGGG | |
| | | TTT | |
| # | rs3087560 | AGATCCCTGTTCTGAAGATCGACTC | 311 |
| | | [C/T]GGTCCATTTTAGAGGCCTCTGGT | |
| | | TC | |
| * | rs515596 | CCAAGTGTAAGAAAGGAATAAGGA | 312 |
| | | T[C/T]CTAGCACGTGCAACATAAGCG | |
| | | ATAC | |
| # | rs7359740 | ACAGGTGTCAAGGGTGAGCTCTGTG | 313 |
| | | [A/G]CGGAAATGGCACTGGGGTATT | |
| | | ATGG | |
| # | rs1944336 | GTCTCTCATATCCATGAACGTCTCT | 314 |
| | | [G/T]CATCACCACTGAAGTCCAAGCC | |
| | | ACC | |
| # | rs8090267 | CACCTCCTTGATAGCCCCCATTCAT | 315 |
| | | [C/T]CATTTATTGAATGACTGTTTACT | |
| | | AC | |
| % | rs534637 | GGACTAGAGGGGGAAGAACTGAAA | 316 |
| | | C[C/T]CAGAGAAAAGTTGGCACAGT | |
| | | GCCAG | |
| * | rs900969 | TGGAGACTTTGTAAACCACTAAGAA | 317 |
| | | [A/G]CCTGGGCTGGGGAACTGTCTA | |
| | | GCCC | |
| * | rs553917 | TCTGATAAATTCCCCTGGTTGTGAA | 318 |
| | | [A/G]CATTGTAGCGCATTCATAACAG | |
| | | CTG | |
| * | rs6507643 | CTCTGTTTGGGGTCTCTGACTTCCC | 319 |
| | | [A/G]CAACACTAAAGGTACTACAGAT | |
| | | GAT | |
| # | rs4793084 | AACCTGAATTAGCATGTTAGCAAGC | 320 |
| | | [G/T]CAGTCCCCTGACCATGTGATGT | |
| | | GAT | |
| # | rs4793085 | TCAATGATGGGGGCATGCATAGCTA | 321 |
| | | [A/C]AGAATATAGGGGTGAGGCCGG | |
| | | GTGC | |
| # | rs2079008 | AATGGTTGCTGTGGAGGGGGAAGA | 322 |
| | | A[A/C]ATAGGAGTAGGGAATGAAGA | |
| | | CAAAA | |
| # | rs7209801 | CCTCAAAGGCCCAGGGTCATGTGAT | 323 |
| | | [A/G]CCATTAAGTTGATCCAATAGCT | |
| | | TCC | |
| * | rs8066822 | AGGAGACGCTGTGCCTGTGTGGCCA | 324 |
| | | [C/T]GTGCCCTCTCTGGGCCTCTGTTT | |
| | | TC | |
| * | rs5036 | CACCACATCACACCCGGGTACCCAC | 325 |
| | | [A/G]AGGTGAGGACCCCAGCCTCCT | |
| | | CCGT | |

* = Allele Frequency >0.15 in Nigerian
# = Allele Frequency >0.15 in Nigerian and Caucasian
% = Allele Frequency >0.15 in Caucasian

Equivalents

Those skilled in the art will recognize, or be able to ascertain using no more than routine experimentation, many equivalents to the specific embodiments of the invention described herein. Such equivalents are intended to be encompassed by the following claims.

1. A method of detecting lineage-specific cells in a biological sample, comprising the step of identifying lineage-specific mRNA in said sample, thereby detecting lineage-specific cells in said sample.

2. A method of detecting lineage-specific cells in a biological sample comprising the step of identifying at least one allelic variant in lineage-specific mRNA in said sample, thereby detecting lineage-specific cells in said sample.

3. A method of detecting lineage-specific cells in a biological sample comprising the step of identifying at least one single nucleotide polymorphism in lineage-specific mRNA in said sample, thereby detecting lineage-specific cells in said sample.

4. The method of claim 2, wherein said at least one allelic variant is in a gene selected from the genes listed in Tables 4, 6, 7, and 8.

5. The method of claim 3, wherein said at least one single nucleotide polymorphism is in a gene selected from the genes listed in Tables 4, 6, 7, and 8.

6. The method of claim 1, wherein said lineage-specific cells are hematopoietic cells.

7. The method claim 6, wherein said hematopoietic cells are erythroid cells.

8. The method of claim 6, wherein said hematopoietic cells are lymphoid cells.

9. The method of claim 6, wherein said hematopoietic cells are myeloid cells.

10. The method of claim 3, wherein said single nucleotide polymorphism is selected from the group listed in Table 9.

11. The method of claim 10, wherein said single nucleotide polymorphism is in a β-globin gene.

12. A method of quantifying donor and recipient lineage-specific cells in a subject following progenitor cell transfer comprising the steps of:
(a) obtaining a biological sample from said subject following progenitor cell transfer; and
(b) identifying and quantifying the presence of one or more donor-derived allelic variants and the presence of one or more recipient-derived allelic variants,
thereby quantifying donor and recipient lineage-specific cells in a subject.

13. A method of detecting lineage-specific chimerism of a subject following progenitor cell transfer comprising the steps of:
(a) obtaining a biological sample from said subject following progenitor cell transfer; and
(b) identifying and quantifying the presence of one or more donor-derived lineage-specific allelic variants and the presence of one or more recipient-derived lineage-specific allelic variants,
thereby detecting lineage-specific chimerism of a subject following progenitor cell transfer.

14. The method of claim 12, wherein said allelic variants are contained within a lineage-specific gene.

15. The method claim 14, wherein said lineage-specific gene is selected from the group of genes listed in Tables 4, 5, 6, and 7.

16. The method of claim 12, wherein said allelic variants are single nucleotide polymorphisms (SNPs).

17. The method of claim 16, wherein said single nucleotide polymorphisms (SNPs) are selected from the group consisting of those SNPs listed in Table 9.

18. The method of claim 12, wherein said lineage-specific allelic variants are expressed by a lineage-specific cell selected from the group consisting of erythroid, lymphoid, or myeloid cells.

19. The method of claim 12, wherein said subject is suffering from a disease or disorder.

20. The method of claim 19, wherein said disease or disorder is associated with reduced levels of β-globin mRNA.

21. The method of claim 19, wherein said disease or disorder is selected from the group consisting of: hemoglobinopathies, hemolytic anemia, hereditary elliptocytosis, hereditary stomatocytosis, Chronic Granulomatous Disease, Chediak-Higashi syndrome, myelodysplasia, acute erythroleukemia, Kostmann's syndrome, infant malignant osteopetrosis, severe combined immunodeficiency, Wiskott-Aldrich syndrome, aplastic anemia, Blackfan Diamond anemia, Gaucher's disease, Hurler's syndrome, Hunter's syndrome, infantile metachromatic leukodystrophy, autoimmune disorders, osteogenesis imperfecta, myocardial injury syndromes, Cystic Fibrosis, hemophilia, Gaucher's disease, cancers associated with oncogenes, diabetes mellitus, organ failure or injury, and cognitive and neurodegenerative disorders.

22. The method of claim 12, wherein said progenitor cell is a stem cell.

23. The method of claim 12, wherein said progenitor cell is a transgenic cell.

24. The method of claim 1, wherein said biological sample is blood.

25. The method of claim 1, wherein said biological sample is bone marrow.

26. The method of claim 1, wherein said lineage-specific mRNA is identified by sequencing.

27. The method of claim 26, wherein said sequencing is pyrosequencing.

28. The method of claim 12, wherein said allelic variants are identified by sequencing.

29. The method of claim 28, wherein said sequencing is pyrosequencing.

30. The method of claim 1, wherein said lineage-specific mRNA is identified by an array-based method.

31. The method of claim 12, wherein said allelic variants are identified by an array-based method.

32. The method of claim 12, wherein said subject is a mammal.

33. The method of claim 32, wherein said mammal is a human.

34. A method of detecting lineage-specific cells in a biological sample, comprising the step of:
(a) isolating mRNA from said biological sample;
(b) reverse transcribing cDNA from said mRNA;
(c) amplifying said cDNA; and
(d) identifying lineage-specific cDNA in said sample.

35. A method of detecting lineage-specific cells in a biological sample comprising the steps of:
(a) ascertaining at least one lineage-specific allelic variant in a target sequence;
(b) isolating mRNA from said biological sample;
(c) reverse transcribing cDNA from said mRNA;
(d) amplifying said at least one allelic variant from said cDNA; and
(e) identifying the at least one lineage-specific allelic variant in step (a) in said sample, thereby detecting lineage-specific cells in said sample.

36. The method of claim 35, wherein said amplification of said cDNA comprises the amplification two or more allelic variants.

37. The method of claim 35, wherein said at least one allelic variant is in a gene selected from the genes listed in Tables 4, 6, 7, and 8.

38. The method of claim 34, wherein said amplification of cDNA amplifies a polymorphic region of a gene or fragment thereof selected from the genes listed in Tables 4, 6, 7, and 8.

39. The method of claim 34, wherein said amplification of cDNA amplifies a β-globin gene or fragment thereof.

40. The method of claim 39, wherein said amplification of the β-globin gene or fragment thereof utilizes the primers set forth as SEQ ID No.: 3 and SEQ ID NO: 5.

41. The method of claim 39, wherein said amplification of the β-globin gene or fragment thereof utilizes the primers set forth as SEQ ID No.: 6 and SEQ ID NO: 8.

42.-54. (canceled)

55. The method of claim 21, wherein said cancers associated with oncogenes are selected from the group, breast, prostate, and colon.

56. The method of claim 21, wherein said organ failure or injury is selected from the group cardiac, brain, lung, liver, renal, prostate and pancreas failure or injury.

57. A method for determining the clinical outcome of a progenitor cell transfer in a subject comprising obtaining a biological sample from said subject and identifying lineage-specific mRNA in said biological sample, wherein a substantial amount of donor-derived lineage-specific allelic variants selected from the group in Table 7 is an indication of poor clinical outcome and a substantial amount of recipient-derived lineage-specific allelic variants selected from the group in Table 7 is an indication of favorable clinical outcome, thereby determining the clinical outcome of a progenitor cell transfer in a subject.

58. A method for determining immune reconstitution in a subject following progenitor cell transfer comprising the steps of obtaining a biological sample from said subject and identifying the identify of at least one lineage-specific allelic variant in said biological sample, to thereby determine immune reconstitution in a subject.
